(19) United States
(12) Patent Application Publication
(10) Pub. No.: US 2025/0197359 A1
(43) Pub. Date: Jun. 19, 2025

(54) CANNABINOID RECEPTOR MODULATING COMPOUNDS

(71) Applicant: The U.S.A., as Represented by the Secretary, Department of Health and Human Services, Bethesda, MD (US)

(72) Inventors: Malliga R. Iyer, Germantown, MD (US); Pinaki Bhattacharjee, Rockville, MD (US); Resat Cinar, Potomac, MD (US); George Kunos, Bethesda, MD (US); Szabolcs Dvoracsko, Rockville, MD (US)

(73) Assignee: The U.S.A., as Represented by the Secretary, Department of Health and Human Services, Bethesda, MD (US)

(21) Appl. No.: 18/846,457

(22) PCT Filed: Mar. 8, 2023
(86) PCT No.: PCT/US2023/014846

§ 371 Date: Sep. 12, 2024

Related U.S. Application Data
(60) Provisional application No. 63/319,642, filed on Mar. 14, 2022.

(51) Int. Cl. C07D 237/04; A61K 31/635; A61K 31/64

(52) U.S. Cl. CPC C07D 237/04; A61K 31/635; A61K 31/64

(57) ABSTRACT

Compounds having useful cannabinoid receptor modulating properties. The compounds may be useful in treating complications arising from metabolic, inflammatory and fibrotic disorders.

### PRIORITY CLAIM

This application claims the benefit of U.S. Provisional Appl. No. 63/319,642, filed Mar. 14, 2022, which is incorporated herein by reference in its entirety.

### ACKNOWLEDGMENT OF GOVERNMENT SUPPORT

This invention was made with government support under project number Z01AA000350-21 awarded by the National Institutes of Health. The government has certain rights in the invention.

### BACKGROUND

Endocannabinoids are lipid signaling molecules that act on the same cannabinoid receptors-CB₁ and CB₂—that recognize and mediate the effects of marijuana. Activation of CB₁ receptors increases appetite, increases the biosynthesis and storage of lipids, inhibits the actions of insulin and leptin, and promotes inflammation and fibrosis, which has led to the development of CB₁ receptor blocking drugs for the treatment of obesity and its metabolic complications, referred to as the metabolic syndrome. The prototype compound rimonabant proved effective in the treatment of the metabolic syndrome, but caused neuropsychiatric side effects, which resulted in its withdrawal from the market and halted further therapeutic development of this class of compounds.

### SUMMARY

Disclosed herein are compounds, or a pharmaceutically acceptable salt or ester thereof, having a structure of:

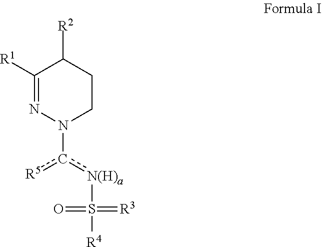

wherein R¹ and R² are each independently aryl, substituted aryl, heteroaryl, or substituted heteroaryl;
R³ is O or NH;
R⁴ is aryl, substituted aryl, heteroaryl, substituted heteroaryl, or N(R¹⁰)(R¹¹), wherein R¹⁰ and R¹¹ are each independently H, optionally-substituted alkyl, optionally-substituted cycloalkyl, halogen, cyano, nitro, hydroxy, optionally-substituted alkoxy, amino, aminocarbonyl, optionally-substituted sulfonyl, optionally-substituted aryl, optionally-substituted heteroaryl, optionally-substituted carboxyl, acyl, optionally-substituted alkenyl, optionally-substituted alkynyl, optionally-substituted phosphonyl, optionally-substituted phosphinyl, aralkyl, or optionally-substituted thiol;
R⁵ is =S, —SR¹², —SeR¹², ═O, —OR¹², —NR¹⁴R¹⁵, —S(O)R¹², —S(O₂)R¹², —Se(O)R¹²—Se(O₂)R¹², or

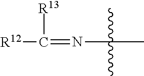

wherein R¹² is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl;
R¹³ is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, allenyl, substituted alkynyl, phosphonyl, substituted phosphonyl, phosphinyl, substituted phosphinyl, boronate, substituted boronate, silyl, substituted silyl, imino, thiol, substituted thiol, selenol, substituted selenol, acyl, or nitrate (ONO₂);
R¹⁴ is H, is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl;
R¹⁵ is H, is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl; or
R¹⁴ and R¹⁵ together form a cyclic ring, a substituted cyclic ring, a heterocyclic ring or a substituted heterocyclic ring;
provided that R⁵ is not —N(H)(methyl), and R⁵ is not ═O when R⁴ is 4-chlorophenyl,

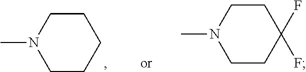

each bond represented by [symbol] is a single or double bond as needed to satisfy valence requirements; and
a is 0 or 1 as needed to satisfy valence requirements.

The foregoing will become more apparent from the following detailed description, which proceeds with reference to the accompanying figures.

### BRIEF DESCRIPTION OF THE DRAWINGS

FIG. 1 shows a synthetic procedure for (A) 1,4,5,6-tetrahydropyridazines and (B) sulfonylated carbamic acid methyl esters.

FIG. 2 shows a synthetic procedure for carboxamides/carboximidamide/carbamimidothioate/carbamimidoselenoate.

FIG. 3 shows a synthetic procedure for halogen substituted 1,4,5,6-tetrahydropyridazines core.

FIG. 4 shows a synthetic procedure for methyl (Z)-3-(4-chlorophenyl)-N-(naphthalen-2-ylsulfonyl)-4-phenyl-5,6-dihydropyridazine-1 (4H)-carbimidothioate.

FIG. 5 shows a synthetic procedure for synthesizing ‘four-arm’ CB1 antagonists.

### DETAILED DESCRIPTION

### Terminology

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

“Acyl” refers to a group having the structure —C(O)R, where R may be, for example, optionally substituted alkyl, optionally substituted aryl, or optionally substituted heteroaryl. “Lower acyl” groups are those that contain one to six carbon atoms. For example, an acyl group may be acetyl.

“Acyloxy” refers to a group having the structure —OC(O)R, where R may be, for example, optionally substituted alkyl, optionally substituted aryl, or optionally substituted heteroaryl. “Lower acyloxy” groups contain one to six carbon atoms. For example, an acyloxy can be a (C₂-C₆)alkanoyloxy which can be, for example, acetoxy, propanoyloxy, butanoyloxy, isobutanoyloxy, pentanoyloxy, or hexanoyloxy.

“Administration” as used herein is inclusive of administration by another person to the subject or self-administration by the subject.

The term “aliphatic” is defined as including alkyl, alkenyl, alkynyl, halogenated alkyl and cycloalkyl groups. A “lower aliphatic” group is a branched or unbranched aliphatic group having from 1 to 10 carbon atoms.

“Alkanediyl,” “cycloalkanediyl,” “aryldiyl,” “alkanearyldiyl” refers to a divalent radical derived from aliphatic, cycloaliphatic, aryl, and alkanearyl hydrocarbons.

“Alkenyl” refers to a cyclic, branched or straight chain group containing only carbon and hydrogen, and contains one or more double bonds that may or may not be conjugated. Alkenyl groups may be unsubstituted or substituted. “Lower alkenyl” groups contain one to six carbon atoms. (C₂-C₆)alkenyl can be, for example, vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl.

The term “alkoxy” refers to a straight, branched or cyclic hydrocarbon configuration and combinations thereof, including from 1 to 20 carbon atoms, preferably from 1 to 8 carbon atoms (referred to as a “lower alkoxy”), more preferably from 1 to 4 carbon atoms, that include an oxygen atom at the point of attachment. An example of an “alkoxy group” is represented by the formula —OR, where R can be an alkyl group, optionally substituted with an alkenyl, alkynyl, aryl, aralkyl, cycloalkyl, halogenated alkyl, alkoxy or heterocycloalkyl group. Suitable alkoxy groups include methoxy, ethoxy, n-propoxy, i-propoxy, n-butoxy, i-butoxy, sec-butoxy, tert-butoxy cyclopropoxy, cyclohexyloxy, pentoxy, 3-pentoxy, or hexyloxy, and the like.

“Alkoxycarbonyl” refers to an alkoxy substituted carbonyl radical, —C(O) OR, wherein R represents an optionally substituted alkyl, aryl, aralkyl, cycloalkyl, cycloalkylalkyl or similar moiety. (C₁-C₆)alkoxycarbonyl can be, for example, methoxycarbonyl, ethoxycarbonyl, propoxycarbonyl, isopropoxycarbonyl, butoxycarbonyl, pentoxycarbonyl, or hexyloxycarbonyl.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 6 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆) cycloalkyl can be cyclopropyl, cyclobutyl, cyclopentyl, or cyclohexyl; (C₃-C₆) cycloalkyl(C₁-C₆)alkyl can be cyclopropylmethyl, cyclobutylmethyl, cyclopentylmcthyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy (C₁-C₆)alkyl can be hydroxymethyl, I-hydroxyethyl, 2-hydroxyethyl, I-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

“Alkynyl” refers to a cyclic, branched or straight chain group containing only carbon and hydrogen, and unless otherwise mentioned typically contains one to twelve carbon atoms, and contains one or more triple bonds. Alkynyl groups may be unsubstituted or substituted. “Lower alkynyl” groups are those that contain two to six carbon atoms. (C₂-C₆)alkynyl, for example, can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

The term “amine” or “amino” refers to a group of the formula —NRR′, where R and R′ can be, independently, hydrogen or an alkyl, alkenyl, alkynyl, acyl, aryl, aralkyl, cycloalkyl, halogenated alkyl, heterocycloalkyl, or carboxyl group. For example, an “alkylamino” or “alkylated amino” refers to —NRR′, wherein at least one of R or R′ is an alkyl. A suitable amine or amino group is acetamido.

The term “aminoalkyl” refers to alkyl groups as defined above where at least one hydrogen atom is replaced with an amino group (e.g, —CH₂—NH₂).

“Aminocarbonyl” alone or in combination, means an amino substituted carbonyl(carbamoyl) radical, wherein the amino radical may optionally be mono- or di-substituted, such as, for example, with alkyl, aryl, acyl, aralkyl, cycloalkyl, cycloalkylalkyl, alkanoyl, alkoxycarbonyl, aralkoxycarbonyl and the like. For example, an aminocarbonyl may be represented by the formula —C(O) NRR′, where R and R′ independently can be, for example, a hydrogen, alkyl, alkenyl, alkynyl, acyl, aryl, aralkyl, cycloalkyl, halogenated alkyl, or heterocycloalkyl group.

An “analog” is a molecule that differs in chemical structure from a parent compound, for example a homolog (differing by an increment in the chemical structure or mass, such as a difference in the length of an alkyl chain or the inclusion of one of more isotopes), a molecular fragment, a structure that differs by onc or more functional groups, or a change in ionization. An analog is not necessarily synthesized from the parent compound. A derivative is a molecule derived from the base structure.

An “animal” refers to living multi-cellular vertebrate organisms, a category that includes, for example, mammals and birds. The term mammal includes both human and non-human mammals.

Similarly, the term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life cycle. Thus, the term subject applies to an organism in utero or in ovo, depending on the organism (that is, whether the organism is a mammal or a bird, such as a domesticated or wild fowl).

The term “aralkyl” refers to an alkyl group wherein an aryl group is substituted for a hydrogen of the alkyl group. An example of an aralkyl group is a benzyl group.

“Aryl” refers to a monovalent unsaturated aromatic carbocyclic group having a single ring (e.g., phenyl) or multiple condensed rings (e.g., naphthyl or anthryl), which can optionally be unsubstituted or substituted. A “heteroaryl group,” is defined as an aromatic group that has at least one heteroatom incorporated within the ring of the aromatic group. Examples of heteroatoms include, but are not limited to, nitrogen, oxygen, sulfur, and phosphorous. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. The aryl or heteroaryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl or heteroaryl group can be unsubstituted.

“Aryloxy” or “heteroaryloxy” refers to a group of the formula —OAr, wherein Ar is an aryl group or a heteroaryl group, respectively.

A “carbonylamino” group may be —N(R)—C(O)—R (wherein each R is independently a substitution group such as, for example, alkyl, alkenyl, alkynyl, acyl, aryl, aralkyl, cycloalkyl, halogenated alkyl, or heterocycloalkyl group, or H). A suitable carbonylamino group is acetamido.

The term “carboxylate” or “carboxyl” refers to the group —COO or —COOH. The carboxyl group can form a carboxylic acid. “Substituted carboxyl” refers to —COOR where R is alkyl, alkenyl, alkynyl, aryl, aralkyl, cycloalkyl, halogenated alkyl, or heterocycloalkyl group. For example, a substituted carboxyl group could be a carboxylic acid ester or a salt thereof (e.g., a carboxylate).

The term “co-administration” or “co-administering” refers to administration of a compound disclosed herein with at least one other therapeutic agent or therapy within the same general time period, and does not require administration at the same exact moment in time (although co-administration is inclusive of administering at the same exact moment in time). Thus, co-administration may be on the same day or on different days, or in the same week or in different weeks. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent and/or lowers the frequency of administering the potentially harmful (e.g., toxic) agent. “Co-administration” or “co-administering” encompass administration of two or more active agents to a subject so that both the active agents and/or their metabolites are present in the subject at the same time. Co-administration includes simultaneous administration in separate compositions, administration at different times in separate compositions, or administration in a composition in which two or more active agents are present.

The term “cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, and the like. The term “heterocycloalkyl group” is a cycloalkyl group as defined above where at least one of the carbon atoms of the ring is substituted with a heteroatom such as, but not limited to, nitrogen, oxygen, sulfur, or phosphorous.

The term “ester” refers to a carboxyl group-containing moiety having the hydrogen replaced with, for example, a C₁₋₆alkyl group (“carboxylC₁₋₆alkyl” or “alkylester”), an aryl or aralkyl group (“arylester” or “aralkylester”) and so on. CO₂C₁₋₃alkyl groups are preferred, such as for example, methylester (CO₂Me), ethylester (CO₂Et) and propylester (CO₂Pr) and includes reverse esters thereof (e.g. —OCOMe, —OCOEt and OCOPr).

“Halo” or “halogen”, as used herein, refers to fluoro, chloro, bromo, and iodo.

The terms “halogenated alkyl” or “haloalkyl group” refer to an alkyl group with one or more hydrogen atoms present on these groups substituted with a halogen (F, Cl, Br, I).

The term “hydroxyl” is represented by the formula —OH.

The term “hydroxyalkyl” refers to an alkyl group that has at least one hydrogen atom substituted with a hydroxyl group. The term “alkoxyalkyl group” is defined as an alkyl group that has at least one hydrogen atom substituted with an alkoxy group described above.

“Inhibiting” refers to inhibiting the full development of a disease or condition. “Inhibiting” also refers to any quantitative or qualitative reduction in biological activity or binding, relative to a control.

“N-heterocyclic” or “N-heterocycle” refers to mono or bicyclic rings or ring systems that include at least one nitrogen heteroatom. The rings or ring systems generally include 1 to 9 carbon atoms in addition to the heteroatom(s) and may be saturated, unsaturated or aromatic (including pseudoaromatic). The term “pseudoaromatic” refers to a ring system which is not strictly aromatic, but which is stabilized by means of delocalization of electrons and behaves in a similar manner to aromatic rings. Aromatic includes pscudoaromatic ring systems, such as pyrrolyl rings.

Examples of 5-membered monocyclic N-heterocycles include pyrrolyl, H-pyrrolyl, pyrrolinyl, pyrrolidinyl, oxazolyl, oxadiazolyl, (including 1,2,3 and 1,2,4 oxadiazolyls) isoxazolyl, furazanyl, thiazolyl, isothiazolyl, pyrazolyl, pyrazolinyl, pyrazolidinyl, imidazolyl, imidazolinyl, triazolyl (including 1,2,3 and 1,3,4 triazolyls), tetrazolyl, thiadiazolyl (including 1,2,3 and 1,3,4 thiadiazolyls), and dithiazolyl. Examples of 6-membered monocyclic N-heterocycles include pyridyl, pyrimidinyl, pyridazinyl, pyrazinyl, piperidinyl, morpholinyl, thiomorpholinyl, piperazinyl, and triazinyl. The heterocycles may be optionally substituted with a broad range of substituents, and preferably with C₁₋₆ alkyl, C₁₋₆ alkoxy, C₂₋₆ alkenyl, C₂₋₆ alkynyl, halo, hydroxy, mercapto, trifluoromethyl, amino, cyano or mono or di(C₁₋₆alkyl)amino. The N-heterocyclic group may be fused to a carbocyclic ring such as phenyl, naphthyl, indenyl, azulenyl, fluorenyl, and anthracenyl.

Examples of 8, 9 and 10-membered bicyclic heterocycles include 1H thieno[2,3-c]pyrazolyl, indolyl, isoindolyl, benzoxazolyl, benzothiazolyl, benzisoxazolyl, benzisothiazolyl, benzimidazolyl, indazolyl, isoquinolinyl, quinolinyl, quinoxalinyl, purinyl, cinnolinyl, phthalazinyl, quinazolinyl, quinoxalinyl, benzotriazinyl, and the like. These heterocycles may be optionally substituted, for example with C₁₋₆ alkyl, C₁₋₆ alkoxy, C₂₋₆ alkenyl, C₂₋₆ alkynyl, halo, hydroxy, mercapto, trifluoromethyl, amino, cyano or mono or di(C₁₋₆alkyl)amino. Unless otherwise defined optionally substituted N-heterocyclics includes pyridinium salts and the N-oxide form of suitable ring nitrogens.

“Selenol” refers to the group —SeH.

The term “substituted selenol” refers to a selenol group having the hydrogen replaced with, for example a C₁₋₆alkyl group (“—Se(C₁₋₆alkyl)”), an aryl (“—Se (aryl)”), or an aralkyl (“—Se (alkyl) (aryl)”) and so on. (C₁-C₆)alkylselenoate, for example, can be methylselenoate, ethylselenoate, propylselenoate, isopropylselenoate, butylselenoate, isobutylselenoate, pentylselenoate, or hexylselenoate.

The term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life cycle. Thus, the term subject applies to an organism in utero or in ovo, depending on the organism (that is, whether the organism is a mammal or a bird, such as a domesticated or wild fowl).

“Substituted” or “substitution” refers to replacement of a hydrogen atom of a molecule or an R-group with one or more additional R-groups. Unless otherwise defined, the term “optionally-substituted” or “optional substituent” as used herein refers to a group which may or may not be further substituted with 1, 2, 3, 4 or more groups, preferably 1, 2 or 3, more preferably 1 or 2 groups. The substituents may be selected, for example, from C₁₋₆alkyl, C₂₋₆alkenyl, C₂₋₆alkynyl, C₃-8cycloalkyl, hydroxyl, oxo, C₁₋₆alkoxy, aryloxy, C₁-calkoxyaryl, halo, C₁₋₆alkylhalo (such as CF₃ and CHF₂), C₁₋₆alkoxyhalo (such as OCF₃ and OCHF2), carboxyl, esters, cyano, nitro, amino, substituted amino, disubstituted amino, acyl, ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC1-6alkyl, heterocyclyl and heteroaryl wherein each alkyl, alkenyl, alkynyl, cycloalkyl, aryl and heterocyclyl and groups containing them may be further optionally substituted. Optional substituents in the case N-heterocycles may also include but are not limited to C₁₋₆alkyl i.e. N—C₁₋₃alkyl, more preferably methyl, particularly N-methyl.

“Sulfinyl” refers to the group —S(═O) H.

The term “substituted sulfinyl” or “sulfoxide” refers to a sulfinyl group having the hydrogen replaced with, for example a C₁₋₆alkyl group (“C₁₋₆alkylsulfinyl” or “C₁₋₆alkylsulfoxide”), an aryl (“arylsulfinyl”), an aralkyl (“aralkyl sulfinyl”) and so on. C₁₋₃alkylsulfinyl groups are preferred, such as for example, -SOmethyl, -SOethyl and -SOpropyl.

The term “sulfonyl” refers to the group —SO₂H. The sulfonyl group can be further substituted with a variety of groups to form, for example, sulfonic acids, sulfonamides, sulfonate esters and sulfones.

The term “substituted sulfonyl” refers to a sulfonyl group having the hydrogen replaced with, for example a C₁₋₆alkyl group (“sulfonylC₁₋₆alkyl”), an aryl (“arylsulfonyl”), an aralkyl (“aralkylsulfonyl”), a heteroaryl, a cycloalkyl, a heterocycloalkyl, and so on. SulfonylC₁₋₃alkyl groups are preferred, such as for example, —SO₂Me, —SO₂Et and —SO₂Pr.

The term “sulfonylamido” or “sulfonamide” refers to the group —SO₂NH₂.

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition.

“Thiol” refers to the group —SH.

The term “substituted thiol” refers to a thiol group having the hydrogen replaced with, for example a C₁₋₆alkyl group (“—S(C₁₋₆alkyl)”), an aryl (“—S(aryl)”), or an aralkyl (“—S(alkyl) (aryl)”) and so on. (C₁-C₆)alkylthio, for example, can be methylthio, ethylthio, propylthio, isopropylthio, butylthio, isobutylthio, pentylthio, or hexylthio.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease, for example, in a subject who is at risk for a disease. A “prophylactic” treatment is a treatment administered to a subject who does not exhibit signs of a disease or exhibits only early signs for the purpose of decreasing the risk of developing a pathology or condition, or diminishing the severity of a pathology or condition.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

“Pharmaceutically acceptable esters” includes those derived from compounds described herein that are modified to include a carboxyl group. An in vivo hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C.sub.1-4 alkyl, or C.sub.1-4 alkoxy) or amino); sulphonate esters, such as alkyl- or aralkylsulphonyl (for example, mcthancsulphonyl); or amino acid esters (for example, L-valyl or L-isoleucyl). A “pharmaceutically acceptable ester” also includes inorganic esters such as mono-, di-, or tri-phosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and especially valyl.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “addition salt” as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary aminc has a positively charged nitrogen. Pharmaceutically acceptable counterions include chloro, bromo, iodo, trifluoroacetate and acetate. The counterion of choice can be introduced using ion exchange resins.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. For a general discussion of prodrugs involving esters see Svensson and Tunek, Drug Metabolism Reviews 165 (1988) and Bundgaard, Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group. In particular examples, a prodrug is a lower alkyl phosphonate ester, such as an isopropyl phosphonate ester.

Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999.

In general, protecting groups are removed under conditions that will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof.

The presently disclosed compounds can have at least one asymmetric center or geometric center, cis-trans center (C=C, C═N). All chiral, diasteromeric, racemic, meso, rotational, conformational and geometric isomers of the structures are intended unless otherwise specified. The compounds can be isolated as a single isomer or as mixture of isomers by methods utilizing specific chiral resolving agents like quinine, Chiral HPLC, SFC (super critical fluid chromatography) etc. All tautomers of the compounds are also considered part of the disclosure. The presently disclosed compounds also include all isotopes of atoms present in the compounds, which can include, but are not limited to, deuterium, tritium, ¹³C, ¹⁸F, stable and radioisotope, etc

### Compounds

The compounds disclosed herein have useful cannabinoid receptor modulating properties. The compounds may be useful in treating complications arising from metabolic, inflammatory and fibrotic disorders.

For example, disclosed herein are novel peripherally restricted cannabinoid receptor mediating compounds for the treatment of, for example, fibrosis (e.g, liver fibrosis), diabetes, obesity and liver cancer.

The cannabinoid receptor may be CB₁ and/or CB₂ receptors. The compounds may be essentially non-selective for CB₁ versus CB₂, or show selectivity for either the CB₁ receptor or the CB₂ receptor. In a preferred embodiment, the cannabinoid receptor mediating compounds are selective of CB₁ receptors.

In certain embodiments, the cannabinoid receptor mediating compounds are cannabinoid receptor inverse agonists, particularly CB₁ inverse agonists. In certain embodiments, the cannabinoid receptor mediating compounds are neutral antagonists. A CB₁ inverse agonist is a drug that on its own produces an effect opposite to that of a CB₁ agonist, and is also able to block the effect of a CB₁ agonist. In contrast, a CB₁ neutral antagonist can only do the latter (i.e. blocking the effect of a CB₁ agonist), but has no effect on its own. CB₁ inverse agonism is usually documented by the ability of a drug to decrease GTPgammaS binding and/or to increase adenylate cyclase activity. The compounds may show functional bias for GTPgammaS or β-Arrestin or activity for both GTPgammaS and β-Arrestin.

In certain embodiments, the compounds preferentially target CB₁ receptors in peripheral tissue (e.g., adipose tissue, liver, muscle, lung, kidney, macrophages, pancreatic beta cells and gastrointestinal tract), while not interacting with CB₁ receptors in brain tissue. Peripherally-mediated effects are maintained, but CNS side effects are minimal or non-existent.

There is evidence that the metabolic effects of endocannabinoids are mediated, at least in part, by CB₁ receptors in peripheral tissues, whereas the neuropsychiatric side effects are mediated by CB receptor in the brain. This suggests CB₁ receptor blocking drugs with reduced ability to penetrate the brain would cause fewer if any neuropsychiatric side effects while retaining some or most of their metabolic benefits. As to limited metabolic efficacy of CB₁ receptor blocking drugs, this could be improved by the design of dual activity compounds that act on more than one target in the cell to influence the same metabolic process. As an example, such secondary targets could include, but not limited to, the enzyme inducible nitric oxide synthase (iNOS) or adenosine monophosphate kinase (AMPK), as suggested by findings that inhibition of iNOS or activation of AMPK improves insulin resistance, and reduces fibrosis and inflammation (Shinozaki S et al., J. Biol. Chem. 2012, 286 (40), 34959-34975; Young R J et al., Bioorg. Med. Chem Let. 2000, 10 (6), 597-600; da Silva Morais A et al., Clin. Sci. 2010, 118 (6), 411-420), Vascular adhesion protein (VAP-1), Aldehyde dehydrogenase (ALDH isoforms), glutathione peroxidase (e.g GPX4). Certain embodiments disclosed herein are CB₁ blocking compounds that have very low brain penetrance, and give rise to metabolites that either inhibit, for example, iNOS or activate AMPK directly. This is now termed Pharmacophore Assisted Novel Targeting of Multi-Mechanisms (PhaNToMM), Fragment Aided Novel Discovery of Multi-Mechanisms (FaNDoMM) or any combination. The acronyms are self-explanatory in that on a main privileged scaffold a pharmacophore or fragment can be attached such that this fragment can modulate a target which can synergize or complement additively to the main scaffold (for, e.g., CB1) to increase/improve efficacy.

In certain embodiments, a peripherally restricted cannabinoid CB₁ receptor mediating compound may be characterized and can be identified from a ratio of maximum concentration in the brain to maximum concentration in plasma which is less than 0.1, as measured in a mouse after intravenous dosing. The preferred peripherally restricted cannabinoid CB₁ receptor mediating compounds have a brain Cₘₐₓ to plasma Cₘₐₓ ratio which is less than 0.05. Especially preferred peripherally restricted cannabinoid receptor mediating compounds have a brain Cₘₐₓ to plasma Cₘₐₓ ratio which is less than 0.025.

In one embodiment, there is disclosed herein a compound, or a pharmaceutically acceptable salt or ester thereof, having a structure of:

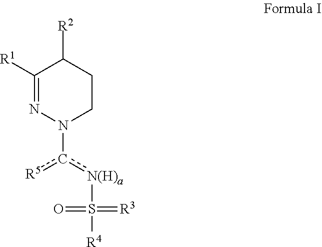

wherein R¹ and R² are each independently aryl, substituted aryl, heteroaryl, or substituted heteroaryl;
R³ is O or NH;
R⁴ is aryl, substituted aryl, heteroaryl, substituted heteroaryl, or N(R¹⁰)(R¹¹), wherein R¹⁰ and R¹¹ are each independently H, optionally-substituted alkyl, optionally-substituted cycloalkyl, halogen, cyano, nitro, hydroxy, optionally-substituted alkoxy, amino, aminocarbonyl, optionally-substituted sulfonyl, optionally-substituted aryl, optionally-substituted heteroaryl, optionally-substituted carboxyl, acyl, optionally-substituted alkenyl, optionally-substituted alkynyl, optionally-substituted phosphonyl, optionally-substituted phosphinyl, aralkyl, or optionally-substituted thiol;
R⁵ is =S, —SR¹², —SeR¹², ═O, —OR¹², —NR¹⁴R¹⁵, —S(O)R¹², —S(O₂) R¹², —Se(O)R¹²—Se(O₂) R¹², or

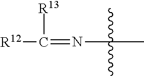

wherein R¹² is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl;
R¹³ is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, allenyl, substituted alkynyl, phosphonyl, substituted phosphonyl, phosphinyl, substituted phosphinyl, boronate, substituted boronate, silyl, substituted silyl, imino, thiol, substituted thiol, selenol, substituted selenol, acyl, or nitrate (ONO₂);
R¹⁴ is H, is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl;
R¹⁵ is H, is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl; or
R¹⁴ and R¹⁵ together form a cyclic ring, a substituted cyclic ring, a heterocyclic ring or a substituted heterocyclic ring;
provided that R⁵ is not —N(H)(methyl), and R⁵ is not ═O when R⁴ is 4-chlorophenyl,

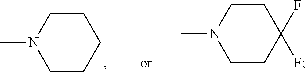

each bond represented by [symbol] is a single or double bond as needed to satisfy valence requirements; and
a is 0 or 1 as needed to satisfy valence requirements.

In certain embodiments, there is disclosed herein a compound, or a pharmaceutically acceptable salt or ester thereof, having a structure of:

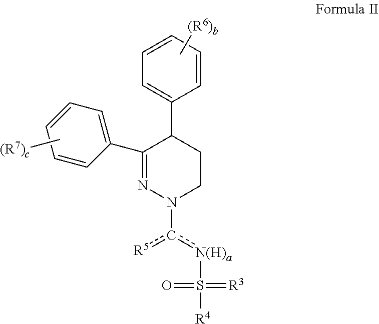

wherein R³, R⁴, R⁵, and subscript a are the same as in formula I;
R⁶ and R⁷ are each independently alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, or substituted thiol;
b is 0 to 5; and
c is 0 to 5.

In certain embodiments, there is disclosed herein a compound, or a pharmaceutically acceptable salt or ester thereof, having a structure of:

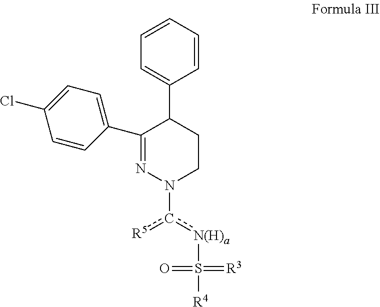

wherein R³, R⁴, R⁵, and subscript a are the same as in formula I.

In certain embodiments, there is disclosed herein a compound, or a pharmaceutically acceptable salt or ester thereof, having a structure of:

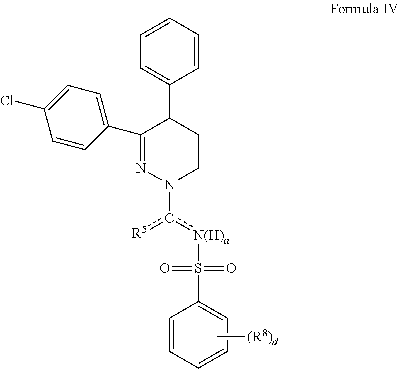

wherein R⁵ and subscript a are the same as in formula I; and
R⁸ is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, or substituted thiol; and d is 0 to 5.

In certain embodiments, there is disclosed herein a compound, or a pharmaceutically acceptable salt or ester thereof, having a structure of:

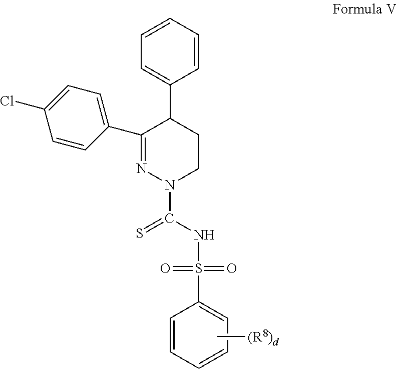

wherein R⁸ and subscript d are the same as in formula IV.

In certain embodiments, there is disclosed herein a compound, or a pharmaceutically acceptable salt or ester thereof, having a structure of:

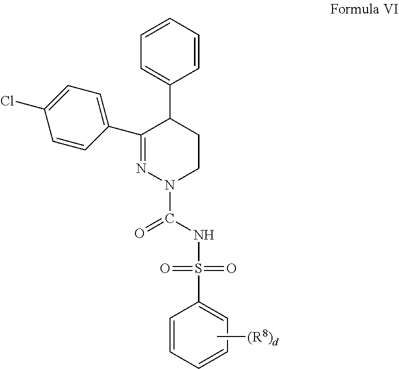

wherein R⁸ and subscript d are the same as in formula IV.

In certain embodiments, there is disclosed herein a compound, or a pharmaceutically acceptable salt or ester thereof, having a structure of:

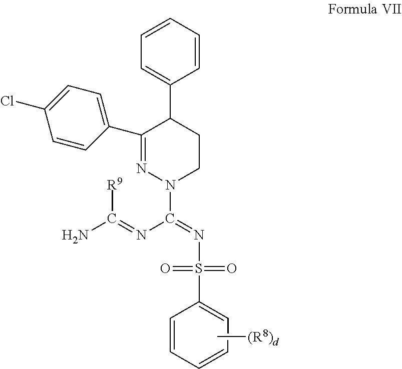

wherein R⁸ and subscript d are the same as in formula IV; and
R⁹ is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, selenol, or substituted selenol.

In certain embodiments, R¹ and R² are each independently phenyl or substituted phenyl, particularly halogen-substituted phenyl. In certain embodiments, R¹ is halogen-substituted phenyl and R² is phenyl.

In certain embodiments, R³ is O.

In certain embodiments, R⁴ is aryl or substituted aryl. In certain embodiments, R⁴ is phenyl, substituted phenyl, naphthyl, or substituted naphthyl. In certain embodiments, R⁴ is phenyl, halogen-substituted phenyl, halogenated alkyl-substituted phenyl, alkoxy-substituted phenyl, halogenated alkoxy-substituted phenyl, cyano-substituted phenyl, or naphthyl.

In certain embodiments, R⁵ is O.

In certain embodiments, R⁵ is S.

In certain embodiments, R⁵ is

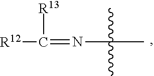

wherein R¹² is amino, substituted amino, acyl, or acetamido.

In certain embodiments, R⁵ is

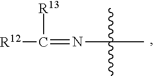

wherein R¹³ is alkyl (e.g., methyl), substituted thiol (e.g., —S(CH₂)ₙCN, —S(CH₂)ₙOH, —S(CH₂)ₙCOOH), wherein n is 0 to 6, or alkyl-substituted thiol (e.g., —SCH₃)), substituted selenol (e.g., —Se(CH₂)ₙCN, —Se(CH₂)ₙOH, —Se(CH₂)ₙ COOH), wherein n is 0 to 6, or alkyl-substituted selenol (e.g., —SeCH₃)), acetyl, 2-iminopiperidinyl, propionyl, cinnamoyl, guanidino, substituted guanidino (e.g., Boc-guanidino), 2-iminopyrrolidinyl, or hydrazine.

In certain embodiments, R⁵ is —NR¹⁴R¹⁵, wherein R¹⁴ is H and R¹⁵ is substituted alkyl or substituted heteroaryl.

In certain embodiments, R⁵ is —NR¹⁴R¹⁵, wherein R¹⁴ is H and R¹⁵ is —(CHR₂)ₙC(═O)OR²⁰, —(CHR²⁰)ₙC(═O)R²⁰, —(CHR²⁰)ₙC(═O)NHR²⁰, or —(CHR²⁰)ₙSO₂NHR²⁰, wherein n is 0 to 6, and R²⁰ is H or alkyl. In certain embodiments, n is 1.

In certain embodiments, R⁵ is —SR¹², wherein R¹² is substituted alkyl or substituted heteroaryl.

In certain embodiments, R⁵ is —SeR¹², wherein R¹² is substituted alkyl or substituted heteroaryl.

In certain embodiments, R⁵ is —S(O)R¹², wherein R¹² is substituted alkyl or substituted heteroaryl.

In certain embodiments, R⁵ is —Se(O)R¹², wherein R¹² is substituted alkyl or substituted heteroaryl.

In certain embodiments, R¹² is —NH₂ or —OCH₃.

In certain embodiments, R¹³ is alkyl (e.g., methyl), alkyl-substituted thiol (e.g., —SCH₃), alkyl-substituted selenol (e.g., —SeCH₃), acetyl, 2-iminopiperidinyl, propionyl, cinnamoyl, guanidino, Boc-guanidino, or 2-iminopyrrolidinyl.

In certain embodiments, b is 0.

In certain embodiments, c is 1.

In certain embodiments, R⁷ is —Cl, —CF₃, —Br, —I, —F, —OCF₃, —CN, —N₃, alkyl, —OH, —NO₂, —(CH₂)alkynyl, alkenyl, —CHF₂, —COR²¹, —COOR²¹, —OR²¹, —(CH₂)ₙCO₂R²¹, —O(CH₂)ₙCO₂R²¹, —OCH₂CH═CHCO₂R²¹, —SO₂NR²¹CH₃, or —NHR²², wherein R²¹ is H or alkyl, R²² is H, acyl, sulfonyl or alkyl, and n is 0 to 6.

In certain embodiments, R⁷ is particularly-C₁.

In certain embodiments, R⁷ is in the para position.

In certain embodiments, d is 0.

In certain embodiments, d is 1.

In certain embodiments, R⁸ is halogen, halogenated alkyl, lower alkyl, nitro, naphthyl, halogenated alkoxy, cyano, or alkoxy.

In certain embodiments, R⁸ is —Cl, —I, —Br, —F, —CH₃, β-naphthyl, —NO₂, —CF₃, —OCF₃, —CN, or —OCH₃.

In certain embodiments, d is 1 and R⁸ is in the para position.

In certain embodiments, d is 1 and R⁸ is in a meta position.

In certain embodiments, d is 1 and R⁸ is in an ortho position.

In certain embodiments, d is 1, R⁸ is in the para position, and is —CF; or —OCF₃.

In certain embodiments, R⁹ is alkyl (e.g., methyl or tert-butyl), heteroaryl (e.g., a N-heteroaryl such as a 5-membered N-heteroaryl), or a substituted aryl (e.g., a halogen-substituted phenyl).

In certain embodiments, the compound is an (−) enantiomer.

In certain embodiments, the agents disclosed herein are hybrid compounds that include (i) a CB₁ receptor mediating scaffold (e.g., an inverse agonist or neutral antagonist) and (ii) a second therapeutic scaffold. In certain embodiments, the —NH—C(NH)R¹-moiety or —N═C(NH₂) R¹-moiety in any of the formulae disclosed herein constitutes at least a portion of the second therapeutic scaffold. In certain embodiments, the second therapeutic scaffold may undergo in vivo cleavage, thereby releasing the second therapeutic scaffold which may retain at least a portion of its therapeutic activity. For example, in the case of metformin as the second therapeutic scaffold, the resulting hybrid compound could have therapeutic efficacy not only due to its blockade of CB₁ receptors, but also due to the release of metformin, a widely used antidiabetic agent, during the in vivo metabolism of the compound. The in vivo cleavage may occur at any location in the body, but typically occurs in the liver, via the action of drug metabolizing enzymes, such as isoforms of cytochrome P450. In certain embodiments, the cleavage occurs at the bond between the —NH—C(NH)R¹-moiety or the —N═C(NH₂)R¹-moiety and the C atom of the carboximidiamide portion of the compound.

Illustrative second therapeutic scaffolds include an antidiabetic agent, an anticancer agent, an antiobesity agent, and an antifibrotic agent.

The second therapeutic scaffold is either implicit as shown below or as an explicit attachment at the unsubstituted nitrogen end, or at the S, Se or O end:

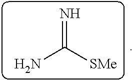

In certain embodiments, the compounds disclosed herein have improved chemical stability resulting in a plasma half-life in the 1-16 hours range, more particularly 4-8 hours range.

In certain embodiments, the agents disclosed herein are hybrid compounds that include (i) a CB₁ receptor mediating scaffold (e.g., an inverse agonist or neutral antagonist) and (ii) a diagnostic agent or entity. In certain embodiments, the —NH—C(NH)R¹-moiety or the —N═C(NH₂) R¹-moiety in any of the formulae disclosed herein constitutes at least a portion of the diagnostic agent or entity (for example, an antibody, biotin tag, ¹⁸F, a PROTAC-tag, or ¹¹C-bearing group). Alternatively, the diagnostic moiety for PET, SPECT imaging, autoradiography, can be obtained by replacing atoms in the X, Y or Q portion of any of the formulae yielding high affinity ligands, with radioisotopic atoms like ²H, ³H, ¹¹C, ¹³N, ¹⁵O, ¹⁸F, ⁷⁵⁻⁷⁷Br, ¹²³⁻¹³¹ I, or ^{99m}Tc. The diagnostic group or entity can be used in the imaging diagnosis of pathologies, like fibrosis, cancer, cardiovascular, metabolic, inflammatory and neurodegenerative diseases. The diagnostic compounds can be used as fluorescent probes, affinity labels, in nuclear medicine, optical imaging, like PET, SPECT etc. These compounds comprise a targeting CB1 scaffold linked to at least a diagnostic signal entity. A CB1 scaffold bearing the diagnostic entity would be capable of targeting at least one marker of a pathologic state, for example proteins, enzymes or cell receptors that are expressed in a pathologic state.

In certain embodiments, the compounds disclosed herein have low or no cytochrome P450 activity meaning that the agents may result in few, if any, drug-to-drug interactions.

In certain embodiments, the compounds disclosed herein have a CB₁R binding affinity in the range of 0.1 to 20 nM, and CB₁/CB₂ selectivity of at least 20-fold, or more particularly 100-fold or greater.

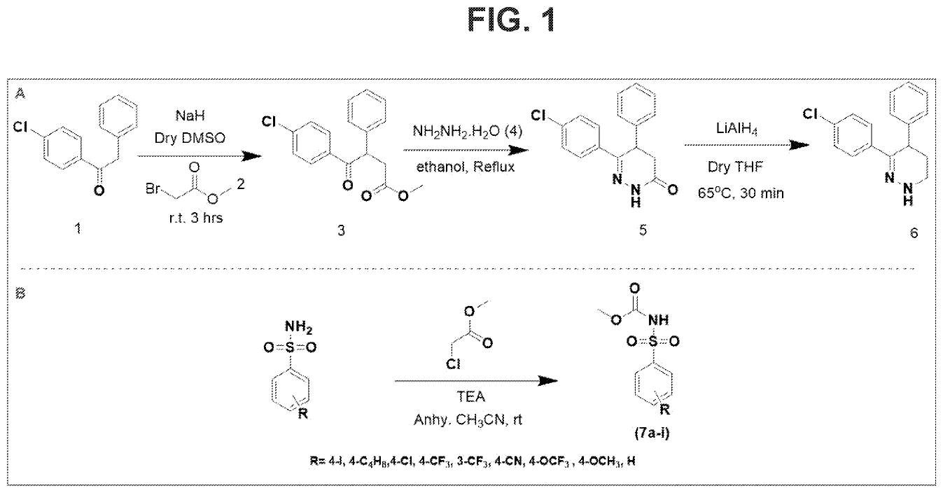

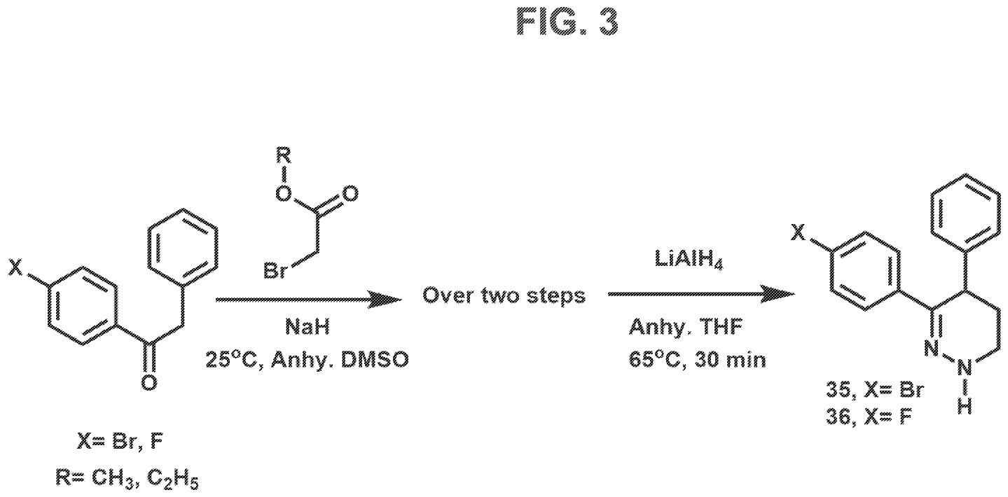

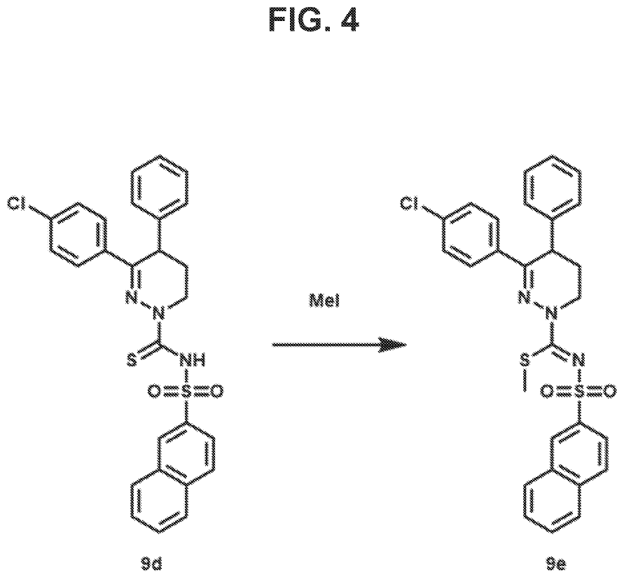

FIGS. 1-5 depict general synthesis methods for making the compounds disclosed herein.

The 1,4,5,6-tetrahydropyridazines have been synthesized as depicted in FIGS. 1-5. Commercially available benzyl 4-chlorophenyl ketone 1 was reacted with ethyl bromoacetate 2 to yield ester adduct 3 in an 37% yield. Further modification of was made by using methyl bromoacetate as a starting materials and yield was improved to 84%. Hydrolysis of 3 led to the corresponding carboxylic acid 4 in 82% yield. Treatment of 4 with hydrazine hydrate gave the 4,5-dihydropyridazin-3-one 5 in a yield of 80%. Whereas direct cyclization of the 3 with hydrazine monohydrate afforded 5 with 85% of yield. 1,4,5,6-tetrahydropyridazine intermediate 6 was obtained from 5 by reduction with LiAlH, in dry THF. The synthetic strategy is based on the coupling of 1,4,5,6-tetrahydropyridazine of general formula 6 with sulfonylated carbamic acid methyl esters of general formula (7a-m), which were obtained from the corresponding arylsulfonamides and methyl chloroformate, to furnish the products 8a-m, respectively, in yields ranging from 78% to 81%.

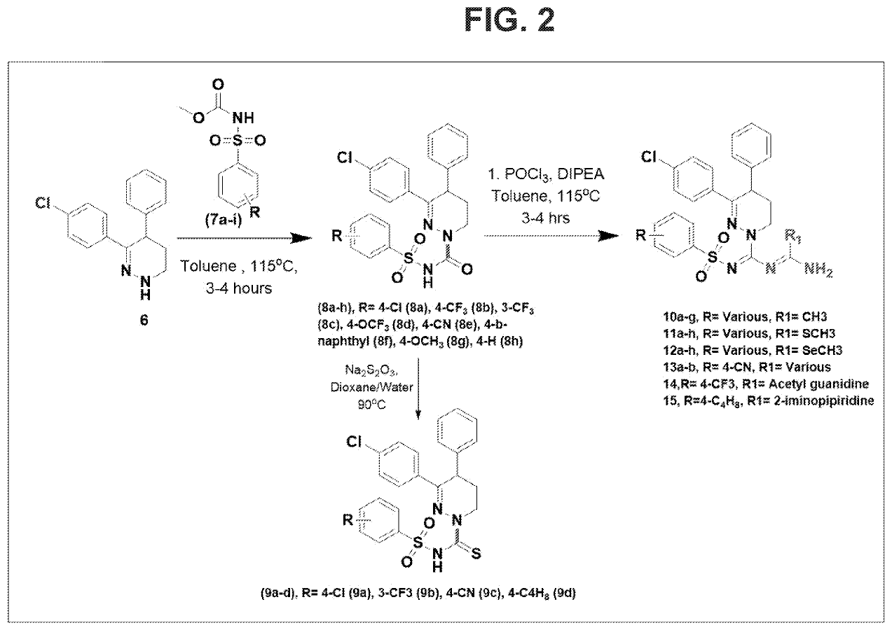

The urea derivatives were converted to the thiourea derivatives (9a-d) by employing sodium thiosulfate. Furthermore, the intermediates 8a-h were chlorinated with phosphorus oxychloride in the presence of DIPEA base and reacted with acetamidine hydrochloride/methyl carbamimidothioate iodide/methyl carbamimidoselenoate iodide and other amines to furnish the target compounds (10-15) (Scheme 2, FIG. 2). The target compounds were evaluated in vitro at the human CB1 receptor, stably expressed into Chinese hamster ovary (CHO) cells by utilizing radioligand binding studies. The CB1 receptor binding data in the amidine-NH₂ series revealed that replacement of the 4-H group in by Cl, CN or CF₃ gave a substantial gain in affinity, whereas OCH₃ substitution did not elicit a clear effect on affinity.

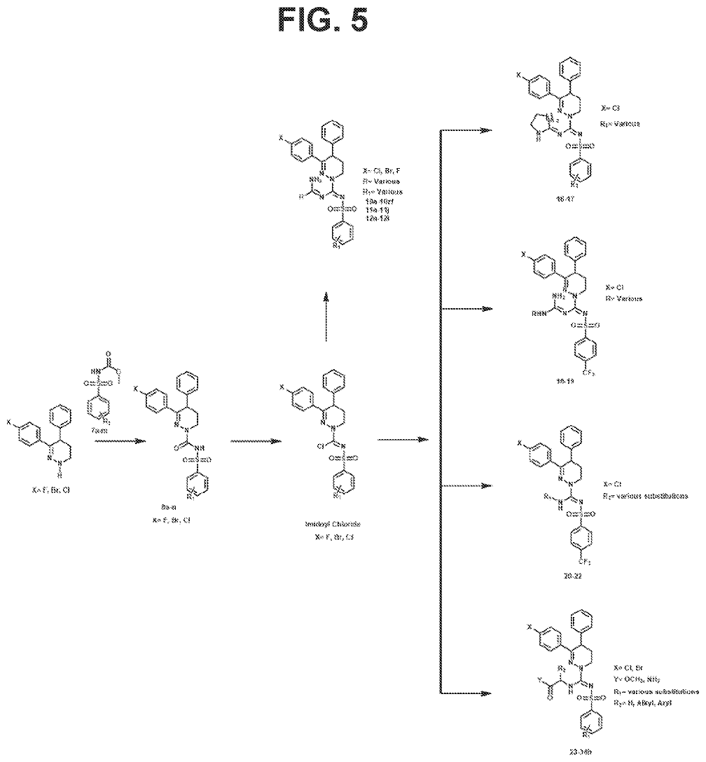

Furthermore, the intermediates urea compounds 8a-m were chlorinated with phosphorus oxychloride in the presence of DIPEA base to form imidoyl chloride intermediate and reacted with acetamidine hydrochloride/methyl carbamimidothioate iodide/methyl carbamimidoselenoate iodide and other amines to furnish the target compounds (10a-34b) (Scheme 2, FIG. 2, Scheme 5, FIG. 5). The urea derivatives were converted to the thiourea derivatives 9a-9d by employing sodium thiosulfate. Compound 8a-b, 8e-f were converted to corresponding thiourea by employing sodium thiosulfate dissolved in water dioxane mixture and the reaction was heated to 85° C. for 2 h. Upon completion of reaction as seen by TLC/LCMS, alkylating agent was added dropwise to the reaction mixture and reaction continued until all the thiourea 9a-d is consumed as seen by TLC/LCMS. The binding constant for the synthesized compounds were determined by using radioligand binding assay in mouse brain membranes as well as the target compounds were evaluated in vitro at the human CB1 receptor, stably expressed into Chinese hamster ovary (CHO) cells as well as mouse brain membrane CB1.

A number of these amino carboxamido adducts with a variety of side chains have been synthesized. This led to interesting CB₁R binding affinities of the synthesized molecules. Several of these racemic (glycine, valine) or diastereomeric molecules have binding affinities in single digit nanomolar range (2 nM to 10 nM) obtained from radioligand binding assay. Furthermore, we investigated the antagonist effect of a few compounds to analyze their capability to modulate CBIR induced by CP55940 in the MPXNomad-CB1 HEK293 cell line. This cell line has been validated measuring cAMP signaling and ß-Arrestin mobilization analyzing the fluorescence intensity of the red cAMPNomad and the green ArresNomad biosensors.

The IC₅₀ for one of the potent compounds [2-(3-(4-chlorophenyl)-4-phenyl-N′-((4-(trifluoromethyl)phenyl)sulfonyl)-1,4,5,6-tetrahydropyridazine-1-carboximidamido)-3-methylbutanamide] was found to be 8.04×10⁻¹⁰ M and 2.27×10⁻¹⁰ M in the red cAMPNomad and the green ArresNomad biosensors, respectively. The corresponding Z′ factors were 0.74 and 0.54 for cAMP and β-arrestin assays, respectively.

A series of synthetic hybrid molecules containing a CB₁R targeting scaffold and a fragment that forms the structural component for iNOS inhibitors were prepared. The resulting compounds are expected to act synergistically via CB1R modulation and iNOS inhibition. Preliminary data (Inducible NOS) showed the significant responses (≥50% inhibition or stimulation for Biochemical assays) in the primary assays are listed below:

TABLE 1

| | | Conc. | |
|---|---|---|---|
| Compound | Species | Criteria | % Response |
| 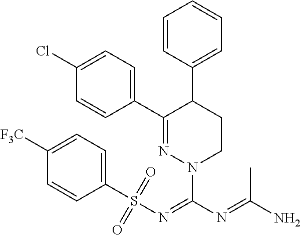 [SMILES: C/C(N)=N\C(=N\S(=O)(=O)c1ccc(C(F)(F)F)cc1)N1CCC(c2ccccc2)C(c2ccc(Cl)cc2)=N1] | Mouse | 10 μM ≥ ±50% | 64% |
| 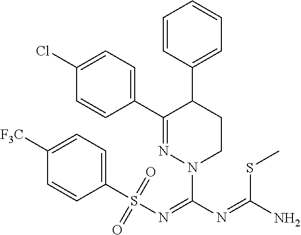 [SMILES: CS/C(N)=N\C(=N\S(=O)(=O)c1ccc(C(F)(F)F)cc1)N1CCC(c2ccccc2)C(c2ccc(Cl)cc2)=N1] | Mouse | 10 μM ≥ ±50% | 56% |
| 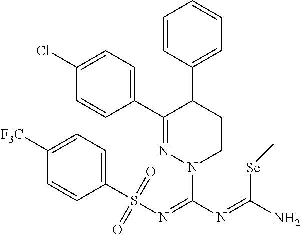 [SMILES: C[Se]/C(N)=N\C(=N\S(=O)(=O)c1ccc(C(F)(F)F)cc1)N1CCC(c2ccccc2)C(c2ccc(Cl)cc2)=N1] | Mouse | 10 μM ≥ ±50% | 64% |

Biochemical assay results are presented as the percent inhibition of specific binding or activity (iNOS, CAMP, beta arrestin).

The MPXNomad-CB1 cell line was used to test substances or examine their ability to interact with Cannabinoid receptor 1 where an agonist that binds to CB1 activates a G protein, which in turn causes CAMP to be released and a cellular response that is then internalized by ß-Arrestin. The assay determined the efficacy to block the activation of the CB1 receptor brought on by CP55940 in the MPXNomad-CB1 HEK293 cell line. AM251 1 μM and 3 UM was utilized as the positive control, CP55940 3 μM was used as the negative control, and vehicle (DMSO) served as the zero control. Treatment with CP55940 3 μM produced an increment in the fluorescence intensity of the red cAMPNomad and the green ArresNomad biosensors. Third generation dual CB1/iNOS inhibitor compound 10b showed a reduction in the fluorescence intensity of approximately a 47% at 1 μM, a 29% at 100 nM and a 19% at 10 nM in the cAMP assay. Instead, the reduction was a 50%, 34% and 22% at 1 μM, 100 nM and 10 nM, respectively in the arrestin assay.

As the pyridazine moiety contains a chiral center at its 4-position, the prepared target compounds of are racemates. To further investigate the stereochemical requirements for binding to the CB1 receptor in this chiral pyridazine series in more detail, the key compounds 10b,10o,10t and 10v were separated into their enantiomers by applying chiral preparative HPLC (SFC) to furnish two optically pure sets of compounds, respectively.

TABLE 2
Caco-2 Pormcability (A-B and B-A permeability data)

| Test | Pₐₚₚ (AB), 10⁻⁶ cm/s | | | | Pₐₚₚ (BA), 10⁻⁶ cm/s | | | | Efflux |
|---|---|---|---|---|---|---|---|---|---|
| Compounds | 1 | 2 | Mean | SD | 1 | 2 | Mean | SD | ratio* |
| 10b | 2.4 | 2.5 | 2.5 | 0.1 | 1.9 | 1.8 | 1.9 | 0.1 | 0.8 |
| 10o | 1.9 | 2.4 | 2.2 | 0.4 | 0.5 | 0.5 | 0.5 | 0.1 | 0.2 |
| 10p | 1.5 | 1.3 | 1.4 | 0.1 | 0.6 | 0.5 | 0.6 | 0.1 | 0.4 |
| 29a | 1.4 | 1.4 | 1.4 | 0.0 | 0.5 | 0.5 | 0.5 | 0.0 | 0.4 |
| 29b | 0.9 | 1.0 | 0.9 | 0.0 | 0.3 | 0.4 | 0.3 | 0.0 | 0.4 |

*Efflux ratio is expressed as the quotient of Pₐₚₚ(BA) to Pₐₚₚ(AB)

TABLE 3
Metabolic Stability in Mouse and Human Liver Microsomes

| | Metabolic Stability in | | Metabolic Stability in | |
|---|---|---|---|---|
| | Mouse Liver Microsomes | | Human Liver Microsomes | |
| Test | t_{1/2}, | Clᵢₙₜ, | t_{1/2}, | Clᵢₙₜ, |
| Compounds | min | μl/min/mg | min | μl/min/mg |
| 10b | 48.8 | 34 | 762.9 | 2 |
| 10o | 73.8 | 23 | 64.2 | 26 |
| 10p | 67.8 | 25 | 287.9 | 6 |
| 29a | 7.1 | 236 | 72.9 | 23 |
| 29b | 1.9 | 883 | 114.6 | 15 |

The compounds showed high microsomal stability in human and mouse microsomes (10b Clint, =34 μl/min/mg, 10o Clint,=23 μl/min/mg, 10p Clint,=25 μl/min/mg in mouse and 10o t1/2=26 μl/min/mg in human) and a moderate intrinsic clearance rate (10b t1/2=48.8 min, 10o Clint,=68 min, 10p t1/2=78 min in mouse and 10o t1/2=64 min in human)

Additional illustrative compounds include:

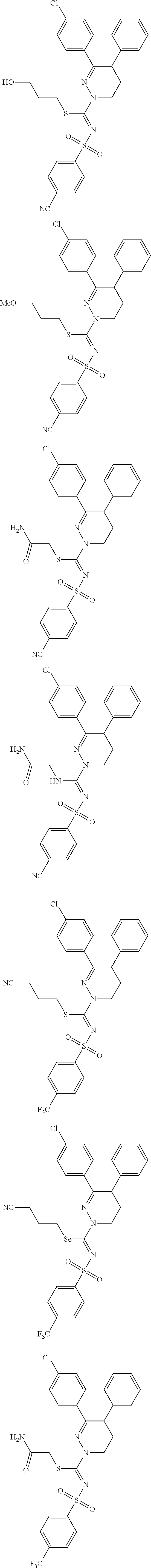

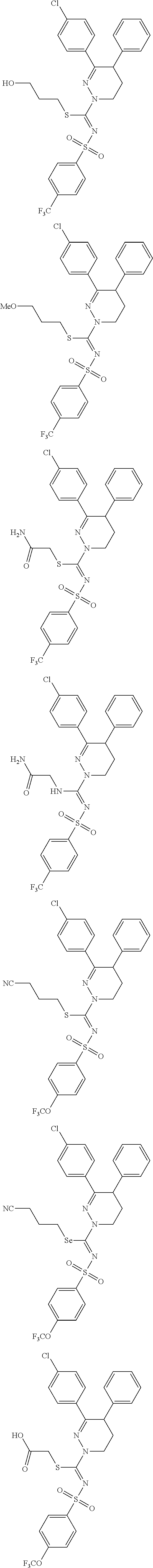

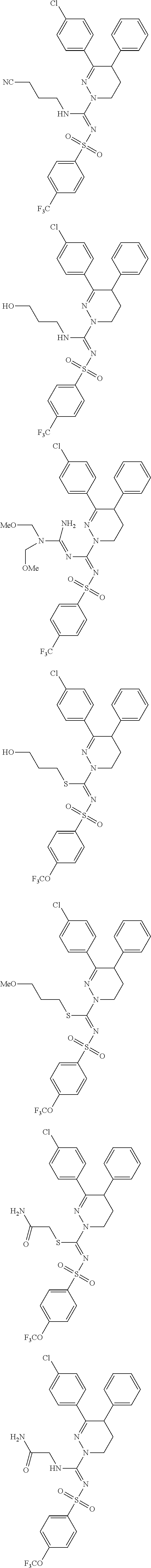

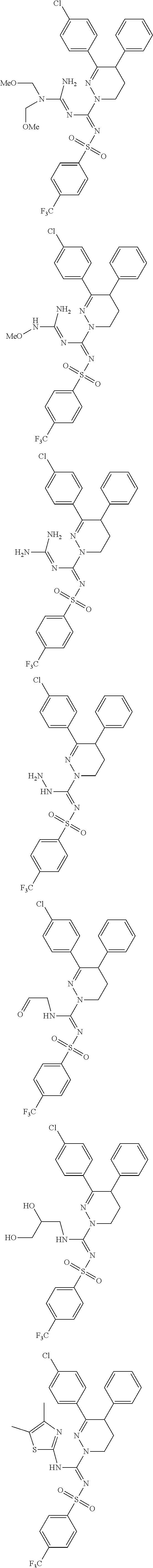

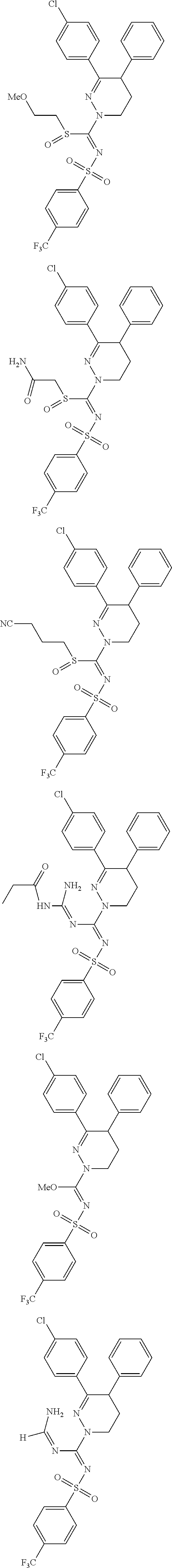

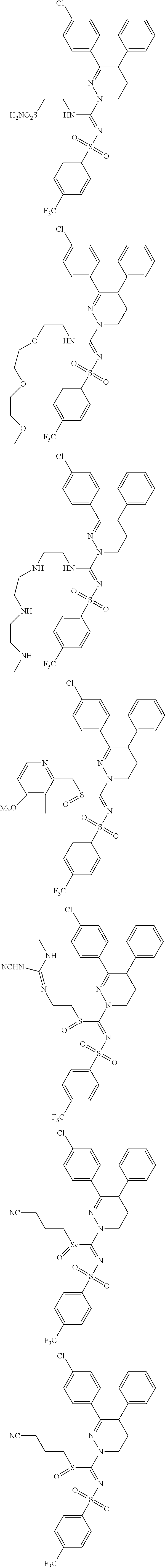

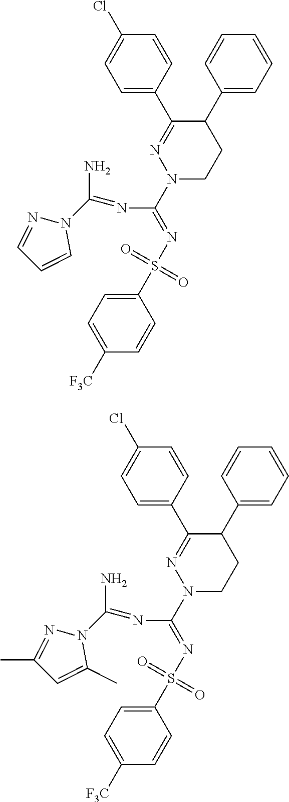 [SMILES: N/C(=N\C(=N/S(=O)(=O)c1ccc(C(F)(F)F)cc1)N1CCC(c2ccccc2)C(c2ccc(Cl)cc2)=N1)n1cccn1 ; Cc1cc(C)n(/C(N)=N/C(=N\S(=O)(=O)c2ccc(C(F)(F)F)cc2)N2CCC(c3ccccc3)C(c3ccc(Cl)cc3)=N2)n1]

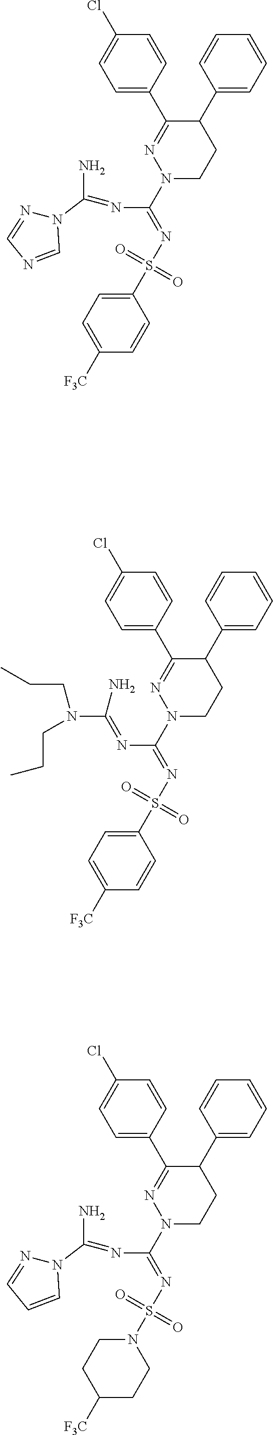 [SMILES: N/C(=N\C(=N/S(=O)(=O)c1ccc(C(F)(F)F)cc1)N1CCC(c2ccccc2)C(c2ccc(Cl)cc2)=N1)n1cncn1 ; CCCN(CCC)/C(N)=N/C(=N\S(=O)(=O)c1ccc(C(F)(F)F)cc1)N1CCC(c2ccccc2)C(c2ccc(Cl)cc2)=N1 ; N/C(=N\C(=N/S(=O)(=O)N1CCC(C(F)(F)F)CC1)N1CCC(c2ccccc2)C(c2ccc(Cl)cc2)=N1)n1cccn1]

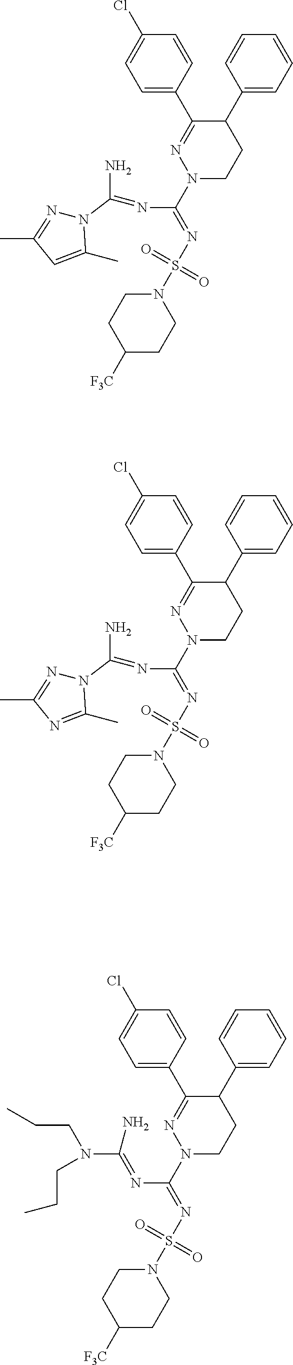 [SMILES: Cc1cc(C)n(/C(N)=N/C(=N\S(=O)(=O)N2CCC(C(F)(F)F)CC2)N2CCC(c3ccccc3)C(c3ccc(Cl)cc3)=N2)n1 ; Cc1nc(C)n(/C(N)=N/C(=N\S(=O)(=O)N2CCC(C(F)(F)F)CC2)N2CCC(c3ccccc3)C(c3ccc(Cl)cc3)=N2)n1 ; CCCN(CCC)/C(N)=N/C(=N\S(=O)(=O)N1CCC(C(F)(F)F)CC1)N1CCC(c2ccccc2)C(c2ccc(Cl)cc2)=N1]

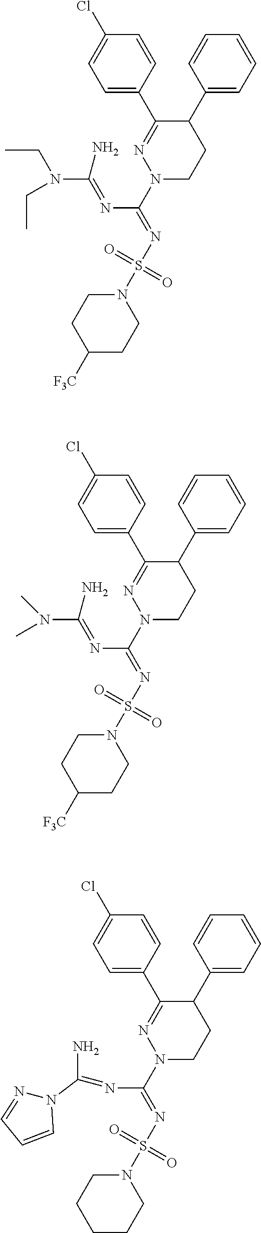 [SMILES: CCN(CC)/C(N)=N/C(=N\S(=O)(=O)N1CCC(C(F)(F)F)CC1)N1CCC(c2ccccc2)C(c2ccc(Cl)cc2)=N1 ; CN(C)/C(N)=N/C(=N\S(=O)(=O)N1CCC(C(F)(F)F)CC1)N1CCC(c2ccccc2)C(c2ccc(Cl)cc2)=N1 ; N/C(=N\C(=N/S(=O)(=O)N1CCCCC1)N1CCC(c2ccccc2)C(c2ccc(Cl)cc2)=N1)n1cccn1]

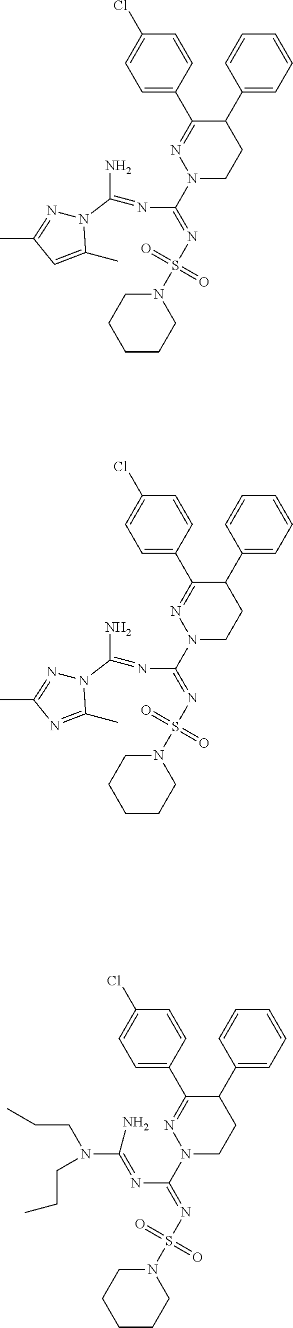 [SMILES: Cc1cc(C)n(/C(N)=N/C(=N\S(=O)(=O)N2CCCCC2)N2CCC(c3ccccc3)C(c3ccc(Cl)cc3)=N2)n1 ; Cc1nc(C)n(/C(N)=N/C(=N\S(=O)(=O)N2CCCCC2)N2CCC(c3ccccc3)C(c3ccc(Cl)cc3)=N2)n1 ; CCCN(CCC)/C(N)=N/C(=N\S(=O)(=O)N1CCCCC1)N1CCC(c2ccccc2)C(c2ccc(Cl)cc2)=N1]

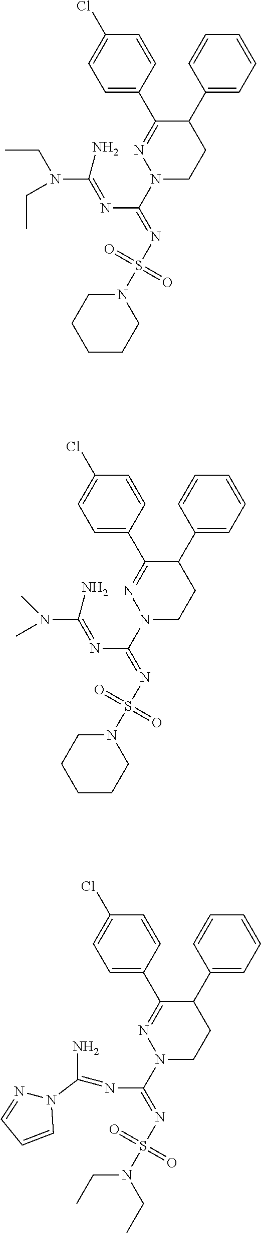 [SMILES: CCN(CC)/C(N)=N/C(=N\S(=O)(=O)N1CCCCC1)N1CCC(c2ccccc2)C(c2ccc(Cl)cc2)=N1 ; CN(C)/C(N)=N/C(=N\S(=O)(=O)N1CCCCC1)N1CCC(c2ccccc2)C(c2ccc(Cl)cc2)=N1 ; CCN(CC)S(=O)(=O)/N=C(\N=C(/N)n1cccn1)N1CCC(c2ccccc2)C(c2ccc(Cl)cc2)=N1]

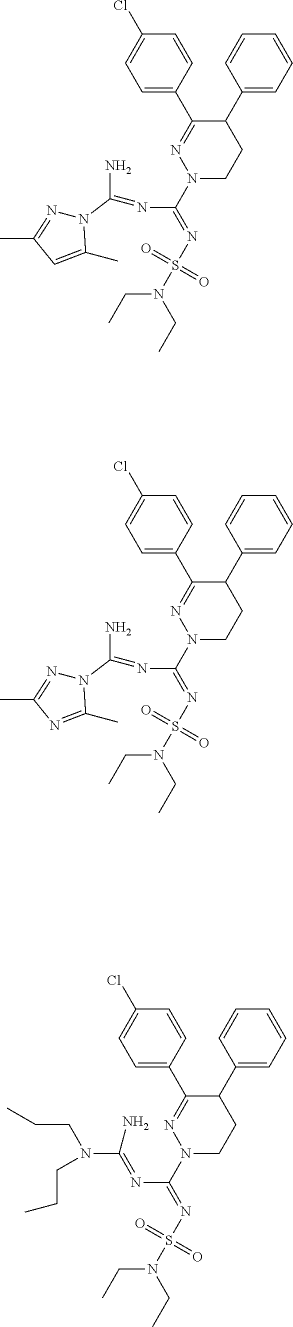 [SMILES: CCN(CC)S(=O)(=O)/N=C(\N=C(/N)n1nc(C)cc1C)N1CCC(c2ccccc2)C(c2ccc(Cl)cc2)=N1 ; CCN(CC)S(=O)(=O)/N=C(\N=C(/N)n1nc(C)nc1C)N1CCC(c2ccccc2)C(c2ccc(Cl)cc2)=N1 ; CCCN(CCC)/C(N)=N/C(=N\S(=O)(=O)N(CC)CC)N1CCC(c2ccccc2)C(c2ccc(Cl)cc2)=N1]

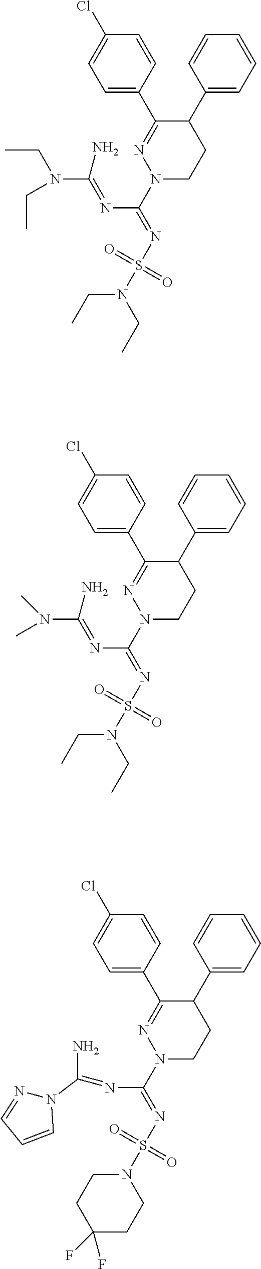 [SMILES: CCN(CC)/C(N)=N/C(=N\S(=O)(=O)N(CC)CC)N1CCC(c2ccccc2)C(c2ccc(Cl)cc2)=N1 ; CCN(CC)S(=O)(=O)/N=C(\N=C(/N)N(C)C)N1CCC(c2ccccc2)C(c2ccc(Cl)cc2)=N1 ; N/C(=N\C(=N/S(=O)(=O)N1CCC(F)(F)CC1)N1CCC(c2ccccc2)C(c2ccc(Cl)cc2)=N1)n1cccn1]

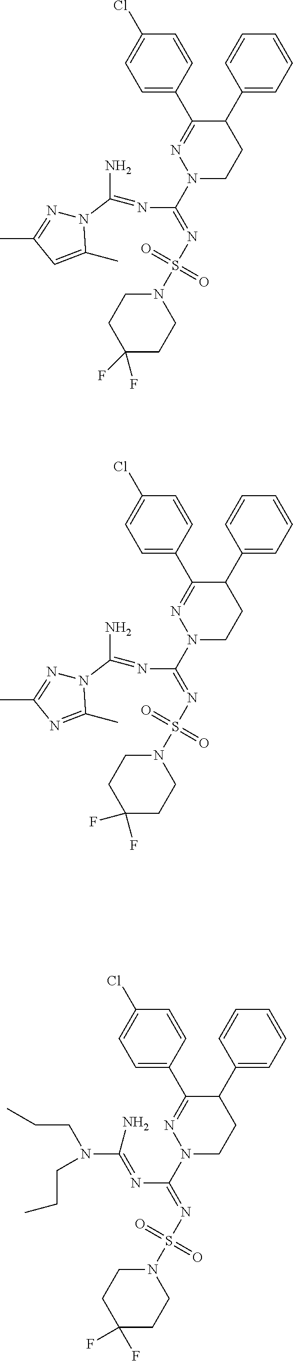 [SMILES: Cc1cc(C)n(/C(N)=N/C(=N\S(=O)(=O)N2CCC(F)(F)CC2)N2CCC(c3ccccc3)C(c3ccc(Cl)cc3)=N2)n1 ; Cc1nc(C)n(/C(N)=N/C(=N\S(=O)(=O)N2CCC(F)(F)CC2)N2CCC(c3ccccc3)C(c3ccc(Cl)cc3)=N2)n1 ; CCCN(CCC)/C(N)=N/C(=N\S(=O)(=O)N1CCC(F)(F)CC1)N1CCC(c2ccccc2)C(c2ccc(Cl)cc2)=N1]

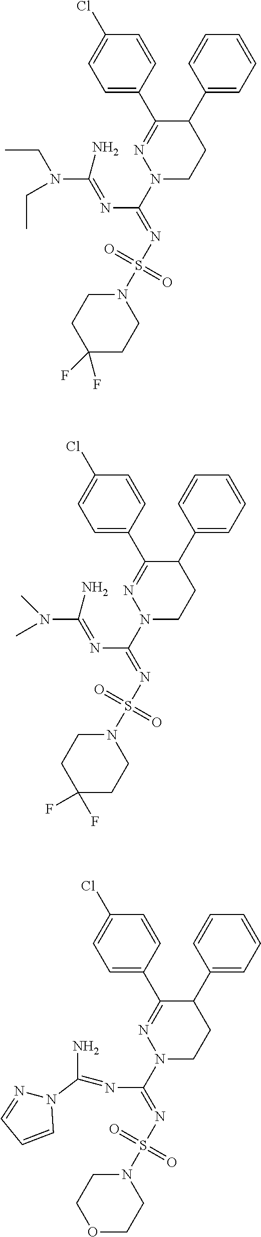 [SMILES: CCN(CC)/C(N)=N/C(=N\S(=O)(=O)N1CCC(F)(F)CC1)N1CCC(c2ccccc2)C(c2ccc(Cl)cc2)=N1 ; CN(C)/C(N)=N/C(=N\S(=O)(=O)N1CCC(F)(F)CC1)N1CCC(c2ccccc2)C(c2ccc(Cl)cc2)=N1 ; N/C(=N\C(=N/S(=O)(=O)N1CCOCC1)N1CCC(c2ccccc2)C(c2ccc(Cl)cc2)=N1)n1cccn1]

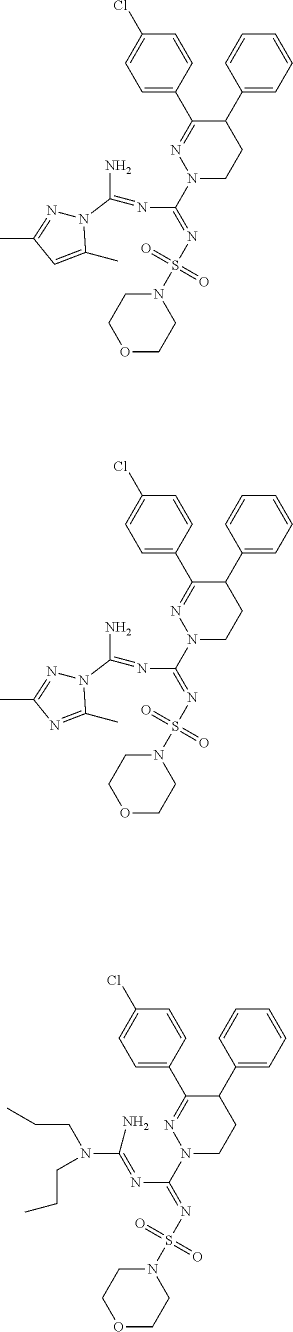 [SMILES: Cc1cc(C)n(/C(N)=N/C(=N\S(=O)(=O)N2CCOCC2)N2CCC(c3ccccc3)C(c3ccc(Cl)cc3)=N2)n1 ; Cc1nc(C)n(/C(N)=N/C(=N\S(=O)(=O)N2CCOCC2)N2CCC(c3ccccc3)C(c3ccc(Cl)cc3)=N2)n1 ; CCCN(CCC)/C(N)=N/C(=N\S(=O)(=O)N1CCOCC1)N1CCC(c2ccccc2)C(c2ccc(Cl)cc2)=N1]

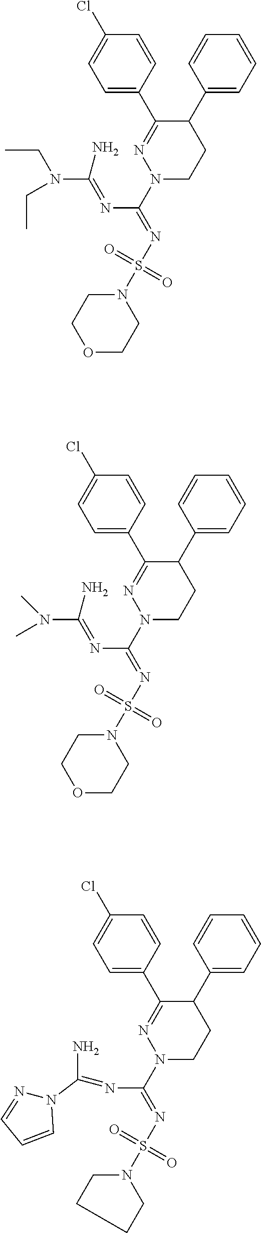 [SMILES: CCN(CC)/C(N)=N/C(=N\S(=O)(=O)N1CCOCC1)N1CCC(c2ccccc2)C(c2ccc(Cl)cc2)=N1 ; CN(C)/C(N)=N/C(=N\S(=O)(=O)N1CCOCC1)N1CCC(c2ccccc2)C(c2ccc(Cl)cc2)=N1 ; N/C(=N\C(=N/S(=O)(=O)N1CCCC1)N1CCC(c2ccccc2)C(c2ccc(Cl)cc2)=N1)n1cccn1]

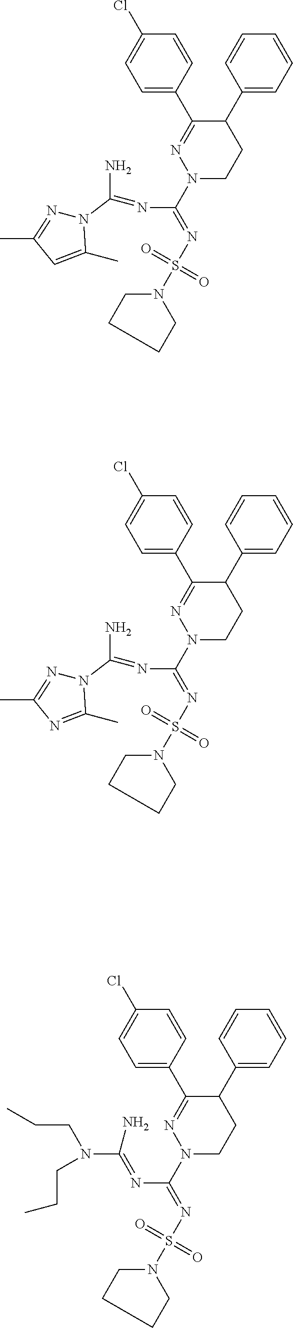 [SMILES: Cc1cc(C)n(/C(N)=N/C(=N\S(=O)(=O)N2CCCC2)N2CCC(c3ccccc3)C(c3ccc(Cl)cc3)=N2)n1 ; Cc1nc(C)n(/C(N)=N/C(=N\S(=O)(=O)N2CCCC2)N2CCC(c3ccccc3)C(c3ccc(Cl)cc3)=N2)n1 ; CCCN(CCC)/C(N)=N/C(=N\S(=O)(=O)N1CCCC1)N1CCC(c2ccccc2)C(c2ccc(Cl)cc2)=N1]

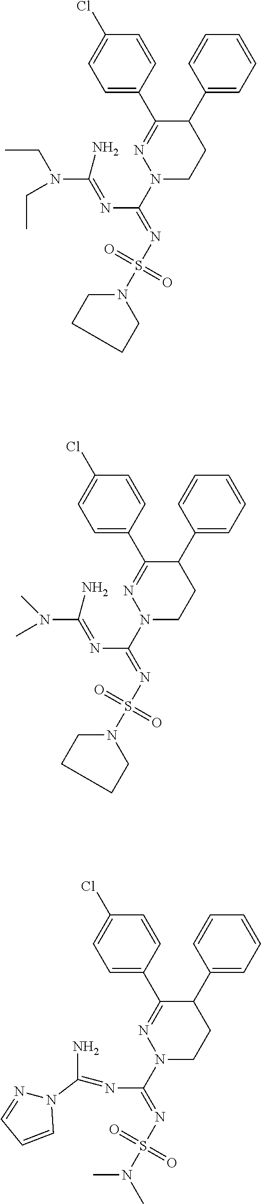 [SMILES: CCN(CC)/C(N)=N/C(=N\S(=O)(=O)N1CCCC1)N1CCC(c2ccccc2)C(c2ccc(Cl)cc2)=N1 ; CN(C)/C(N)=N/C(=N\S(=O)(=O)N1CCCC1)N1CCC(c2ccccc2)C(c2ccc(Cl)cc2)=N1 ; CN(C)S(=O)(=O)/N=C(\N=C(/N)n1cccn1)N1CCC(c2ccccc2)C(c2ccc(Cl)cc2)=N1]

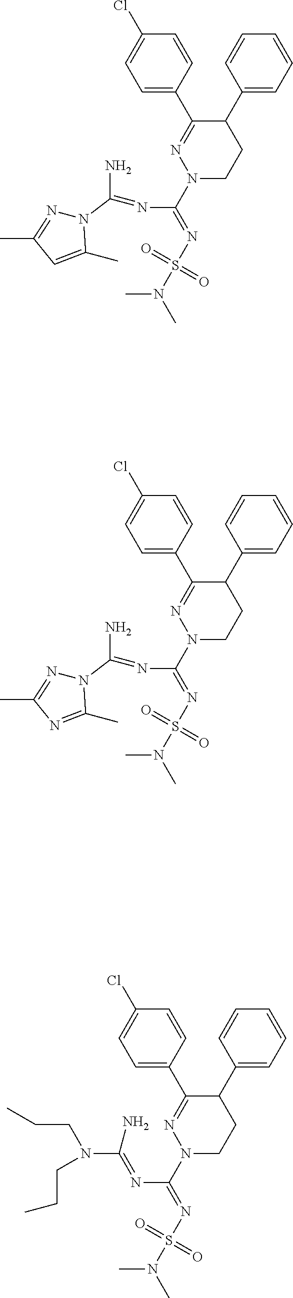 [SMILES: Cc1cc(C)n(/C(N)=N/C(=N\S(=O)(=O)N(C)C)N2CCC(c3ccccc3)C(c3ccc(Cl)cc3)=N2)n1 ; Cc1nc(C)n(/C(N)=N/C(=N\S(=O)(=O)N(C)C)N2CCC(c3ccccc3)C(c3ccc(Cl)cc3)=N2)n1 ; CCCN(CCC)/C(N)=N/C(=N\S(=O)(=O)N(C)C)N1CCC(c2ccccc2)C(c2ccc(Cl)cc2)=N1]

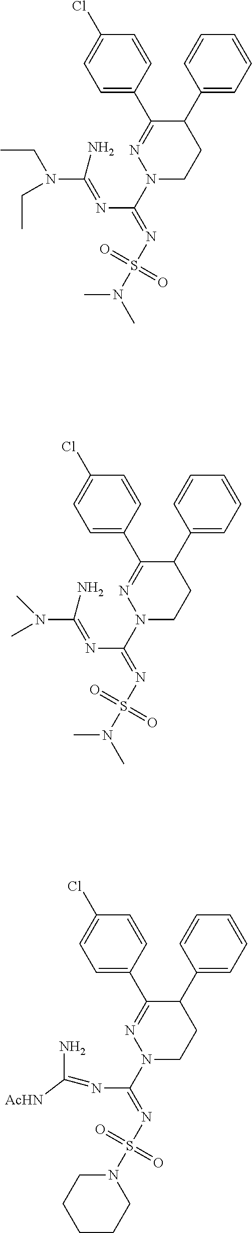 [SMILES: CCN(CC)/C(N)=N/C(=N\S(=O)(=O)N(C)C)N1CCC(c2ccccc2)C(c2ccc(Cl)cc2)=N1 ; CN(C)/C(N)=N/C(=N\S(=O)(=O)N(C)C)N1CCC(c2ccccc2)C(c2ccc(Cl)cc2)=N1 ; CC(=O)N/C(N)=N/C(=N\S(=O)(=O)N1CCCCC1)N1CCC(c2ccccc2)C(c2ccc(Cl)cc2)=N1]

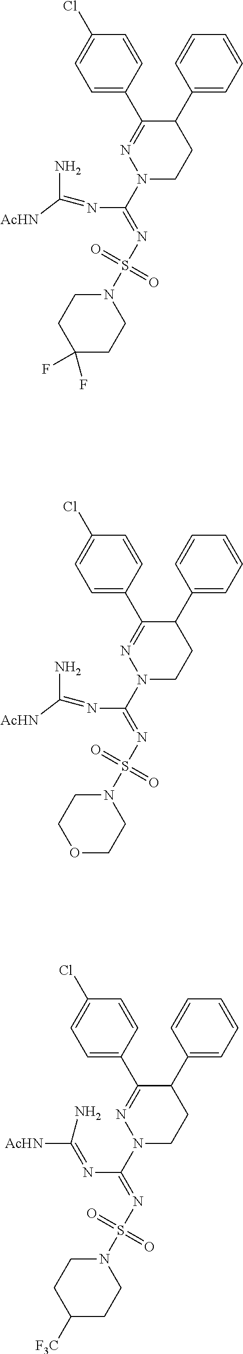 [SMILES: CC(=O)N/C(N)=N/C(=N\S(=O)(=O)N1CCC(F)(F)CC1)N1CCC(c2ccccc2)C(c2ccc(Cl)cc2)=N1 ; CC(=O)N/C(N)=N/C(=N\S(=O)(=O)N1CCOCC1)N1CCC(c2ccccc2)C(c2ccc(Cl)cc2)=N1 ; CC(=O)N/C(N)=N/C(=N\S(=O)(=O)N1CCC(C(F)(F)F)CC1)N1CCC(c2ccccc2)C(c2ccc(Cl)cc2)=N1]

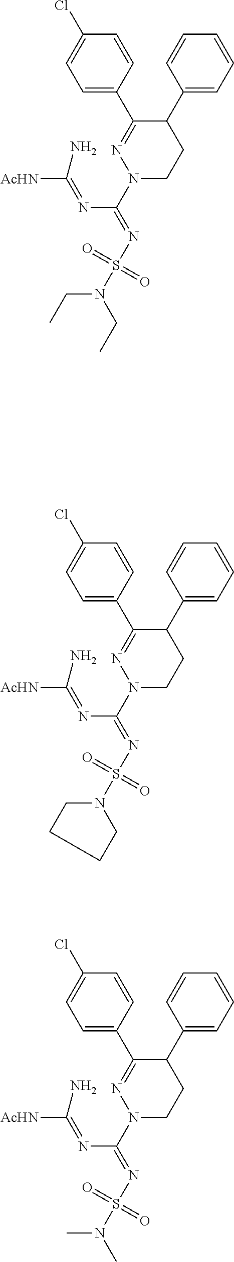 [SMILES: CCN(CC)S(=O)(=O)/N=C(\N=C(/N)NC(C)=O)N1CCC(c2ccccc2)C(c2ccc(Cl)cc2)=N1 ; CC(=O)N/C(N)=N/C(=N\S(=O)(=O)N1CCCC1)N1CCC(c2ccccc2)C(c2ccc(Cl)cc2)=N1 ; CC(=O)N/C(N)=N/C(=N\S(=O)(=O)N(C)C)N1CCC(c2ccccc2)C(c2ccc(Cl)cc2)=N1]

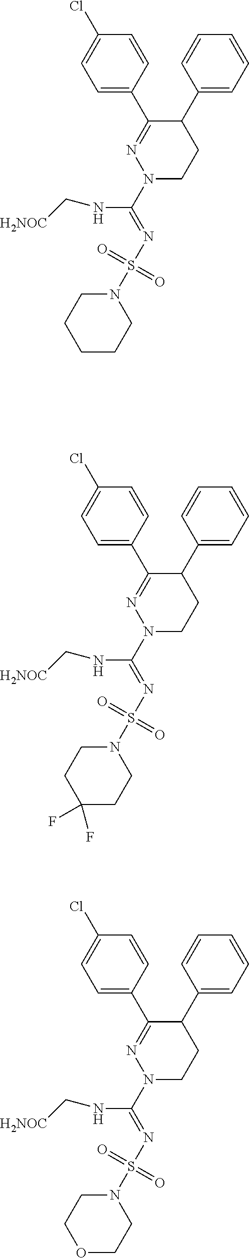 [SMILES: NC(=O)CN/C(=N\S(=O)(=O)N1CCCCC1)N1CCC(c2ccccc2)C(c2ccc(Cl)cc2)=N1 ; NC(=O)CN/C(=N\S(=O)(=O)N1CCC(F)(F)CC1)N1CCC(c2ccccc2)C(c2ccc(Cl)cc2)=N1 ; NC(=O)CN/C(=N\S(=O)(=O)N1CCOCC1)N1CCC(c2ccccc2)C(c2ccc(Cl)cc2)=N1]

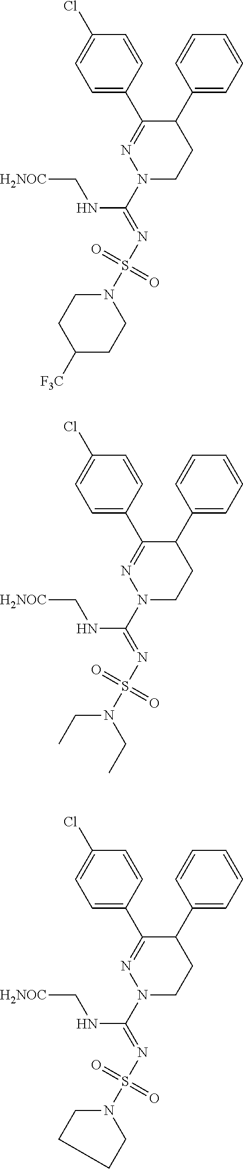 [SMILES: NC(=O)CN/C(=N\S(=O)(=O)N1CCC(C(F)(F)F)CC1)N1CCC(c2ccccc2)C(c2ccc(Cl)cc2)=N1 ; CCN(CC)S(=O)(=O)/N=C(\NCC(N)=O)N1CCC(c2ccccc2)C(c2ccc(Cl)cc2)=N1 ; NC(=O)CN/C(=N\S(=O)(=O)N1CCCC1)N1CCC(c2ccccc2)C(c2ccc(Cl)cc2)=N1]

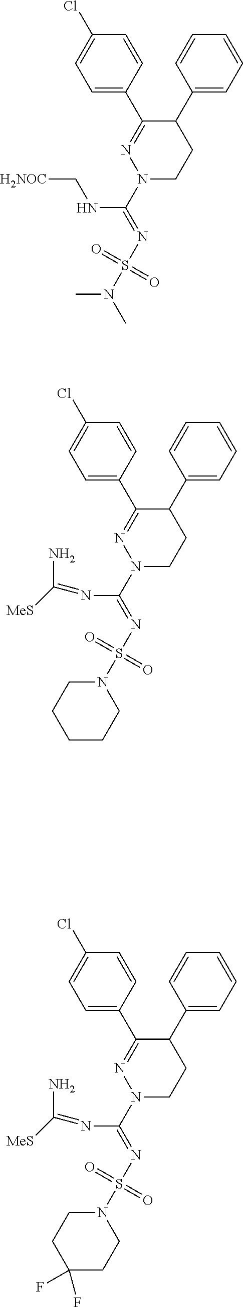 [SMILES: CN(C)S(=O)(=O)/N=C(\NCC(N)=O)N1CCC(c2ccccc2)C(c2ccc(Cl)cc2)=N1 ; CS/C(N)=N/C(=N\S(=O)(=O)N1CCCCC1)N1CCC(c2ccccc2)C(c2ccc(Cl)cc2)=N1 ; CS/C(N)=N/C(=N\S(=O)(=O)N1CCC(F)(F)CC1)N1CCC(c2ccccc2)C(c2ccc(Cl)cc2)=N1]

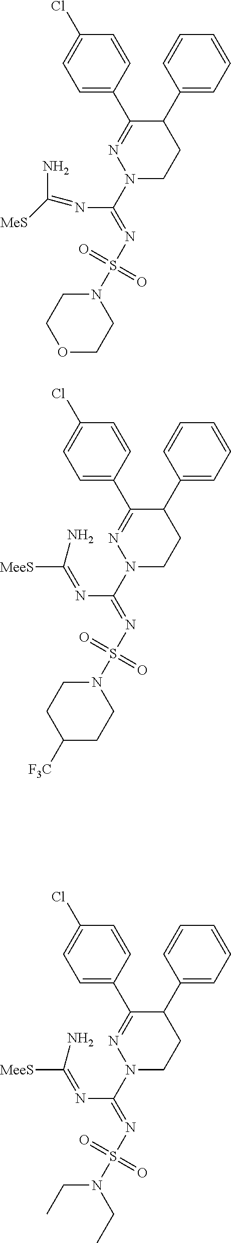

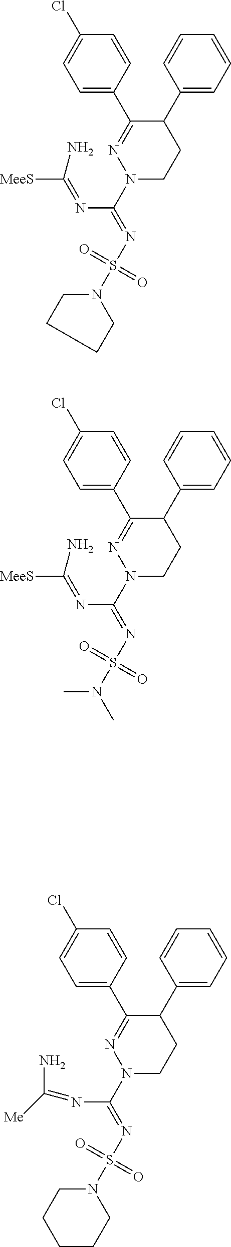

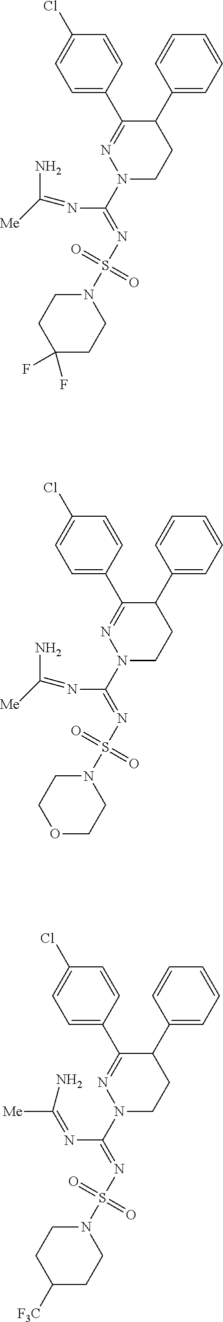 [SMILES: C/C(N)=N/C(=N\S(=O)(=O)N1CCC(F)(F)CC1)N1CCC(c2ccccc2)C(c2ccc(Cl)cc2)=N1 ; C/C(N)=N/C(=N\S(=O)(=O)N1CCOCC1)N1CCC(c2ccccc2)C(c2ccc(Cl)cc2)=N1 ; C/C(N)=N/C(=N\S(=O)(=O)N1CCC(C(F)(F)F)CC1)N1CCC(c2ccccc2)C(c2ccc(Cl)cc2)=N1]

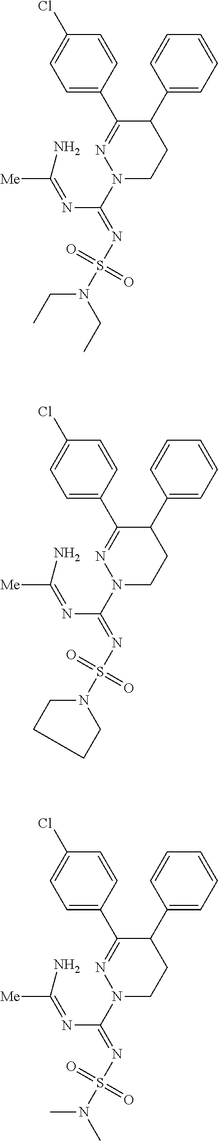 [SMILES: CCN(CC)S(=O)(=O)/N=C(\N=C(\C)N)N1CCC(c2ccccc2)C(c2ccc(Cl)cc2)=N1 ; C/C(N)=N/C(=N\S(=O)(=O)N1CCCC1)N1CCC(c2ccccc2)C(c2ccc(Cl)cc2)=N1 ; C/C(N)=N/C(=N\S(=O)(=O)N(C)C)N1CCC(c2ccccc2)C(c2ccc(Cl)cc2)=N1]

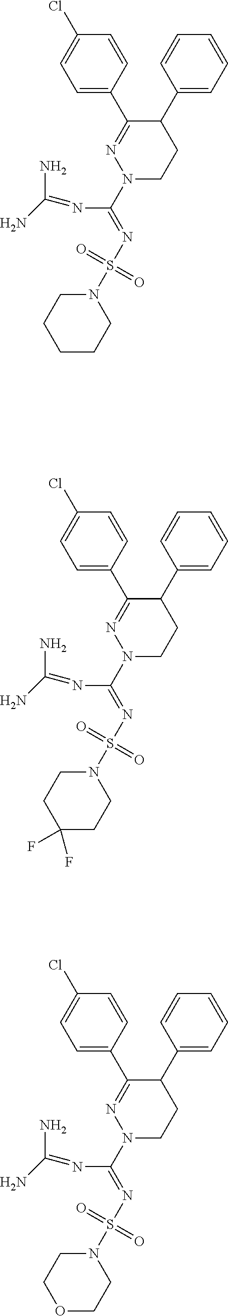 [SMILES: NC(N)=N/C(=N\S(=O)(=O)N1CCCCC1)N1CCC(c2ccccc2)C(c2ccc(Cl)cc2)=N1 ; NC(N)=N/C(=N\S(=O)(=O)N1CCC(F)(F)CC1)N1CCC(c2ccccc2)C(c2ccc(Cl)cc2)=N1 ; NC(N)=N/C(=N\S(=O)(=O)N1CCOCC1)N1CCC(c2ccccc2)C(c2ccc(Cl)cc2)=N1]

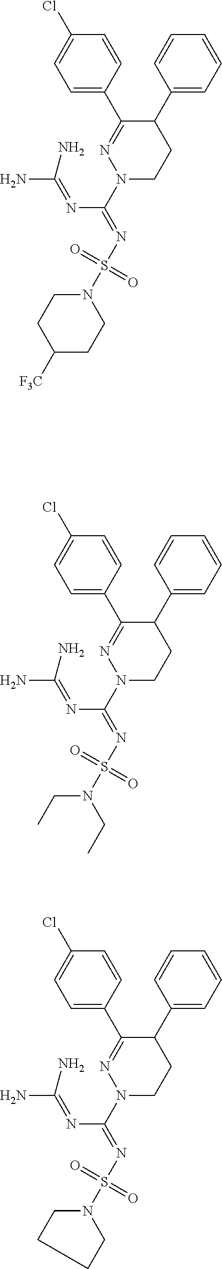 [SMILES: NC(N)=N/C(=N\S(=O)(=O)N1CCC(C(F)(F)F)CC1)N1CCC(c2ccccc2)C(c2ccc(Cl)cc2)=N1 ; CCN(CC)S(=O)(=O)/N=C(\N=C(N)N)N1CCC(c2ccccc2)C(c2ccc(Cl)cc2)=N1 ; NC(N)=N/C(=N\S(=O)(=O)N1CCCC1)N1CCC(c2ccccc2)C(c2ccc(Cl)cc2)=N1]

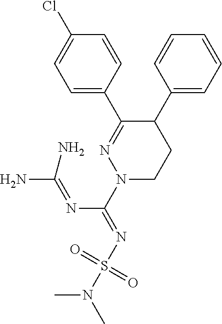 [SMILES: CN(C)S(=O)(=O)/N=C(\N=C(N)N)N1CCC(c2ccccc2)C(c2ccc(Cl)cc2)=N1]

### Compositions and Methods of Use

The peripherally restricted cannabinoid receptor mediating agents disclosed herein are unique in that they may improve all, or at least one, aspect(s) of the metabolic syndrome. They reduce food intake and body weight, reverse insulin and leptin resistance, reverse hepatic steatosis (fatty liver) and improve dyslipidemia. They may be used for treating obesity, diabetes (e.g., type 2 diabetes), and non-alcoholic and alcoholic fatty liver disease (NAFLD/AFLD), the latter being a risk factor for insulin resistance, cirrhosis and liver cancer, dyslipidemias that predispose to arteriosclerotic heart disease, diabetic nephropathy, gout, and fibrosis (e.g., liver fibrosis and pulmonary fibrosis). The agents disclosed herein may be devoid of the psychiatric side effects that prevent the use of globally acting CB₁ antagonists.

The diabetes disorder may be Type 1 diabetes, Type 2 diabetes, inadequate glucose tolerance, and/or insulin resistance.

Also disclosed herein is a method for treating a co-morbidity of obesity. The co-morbidity may be selected from diabetes, Metabolic Syndrome, dementia, heart disease, and cancer. In further embodiments, the co-morbidity is selected from hypertension; gallbladder disease; gastrointestinal disorders; menstrual irregularities; degenerative arthritis; venous statis ulcers; pulmonary hypoventilation syndrome; sleep apnea; snoring; coronary artery disease; arterial sclerotic disease; pseudotumor cerebri; accident proneness; increased risks with surgeries; osteoarthritis; high cholesterol; and, increased incidence of malignancies of the liver, ovaries, cervix, uterus, breasts, prostrate, and gallbladder.

Also disclosed herein is a method for treating diabetic nephropathy, obesity-induced chronic kidney disease.

Also disclosed herein is a method of preventing or reversing the deposition of adipose tissue in a subject. Also disclosed herein is a method of preventing or reversing ectopic deposition of fat in a subject. By preventing or reversing the deposition of adipose tissue, the compounds disclosed herein are expected to reduce the incidence or severity of obesity, thereby reducing the incidence or severity of associated co-morbidities (e.g Praeder-Willi Syndrome, Alstrom Syndrome).

Also disclosed herein is a method for treating the fibrotic disorders such as idiopathic pulmonary fibrosis, Hermansky-Pudlak syndrome associated pulmonary fibrosis, radiation induced pulmonary fibrosis, scleroderma associated organ fibrosis, skin fibrosis, kidney fibrosis, liver fibrosis, chronic graft versus host disease (cGvHD) or any combination thereof.

Also disclosed herein is a method for treating cancer such as liver cancer, breast cancer, and lung cancer.

Also disclosed herein is a method for treating liver impairment from malaria, bacterial (e.g., Tuberculosis), fungal (C. albicans), and viral diseases (e.g., HBV), pulmonary and kidney conditions (rare disease), and skin fibrosis (e.g systemic sclerosis)

Another aspect of the disclosure includes pharmaceutical compositions prepared for administration to a subject and which include a therapeutically effective amount of one or more of the compounds disclosed herein. The therapeutically effective amount of a disclosed compound will depend on the route of administration, the species of subject and the physical characteristics of the subject being treated. Specific factors that can be taken into account include disease severity and stage, weight, diet and concurrent medications. The relationship of these factors to determining a therapeutically effective amount of the disclosed compounds is understood by those of skill in the art.

Pharmaceutical compositions for administration to a subject can include at least one further pharmaceutically acceptable additive such as carriers, thickeners, diluents, buffers, preservatives, surface active agents and the like in addition to the molecule of choice. Pharmaceutical compositions can also include one or more additional active ingredients such as antimicrobial agents, anti-inflammatory agents, anesthetics, and the like. The pharmaceutically acceptable carriers useful for these formulations are conventional. Remington's Pharmaceutical Sciences, by E. W. Martin, Mack Publishing Co., Easton, PA, 19th Edition (1995), describes compositions and formulations suitable for pharmaceutical delivery of the compounds herein disclosed.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical compositions disclosed herein include those formed from pharmaceutically acceptable salts and/or solvates of the disclosed compounds. Pharmaceutically acceptable salts include those derived from pharmaceutically acceptable inorganic or organic bases and acids. Particular disclosed compounds possess at least one basic group that can form acid-base salts with acids. Examples of basic groups include, but are not limited to, amino and imino groups. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with α-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid. In particular, suitable salts include those derived from alkali metals such as potassium and sodium, alkaline earth metals such as calcium and magnesium, among numerous other acids well known in the pharmaceutical art.

Certain compounds include at least one acidic group that can form an acid-base salt with an inorganic or organic base. Examples of salts formed from inorganic bases include salts of the presently disclosed compounds with alkali metals such as potassium and sodium, alkaline earth metals, including calcium and magnesium and the like. Similarly, salts of acidic compounds with an organic base, such as an amine (as used herein terms that refer to amines should be understood to include their conjugate acids unless the context clearly indicates that the free amine is intended) are contemplated, including salts formed with basic amino acids, aliphatic amines, heterocyclic amines, aromatic amines, pyridines, guanidines and amidines. Of the aliphatic amines, the acyclic aliphatic amines, and cyclic and acyclic di- and tri-alkyl amines are particularly suitable for use in the disclosed compounds. In addition, quaternary ammonium counterions also can be used.

Particular examples of suitable amine bases (and their corresponding ammonium ions) for use in the present compounds include, without limitation, pyridine, N,N-dimethylaminopyridine, diazabicyclononane, diazabicycloundecene, N-methyl-N-ethylamine, diethylamine, triethylamine, diisopropylethylamine, mono-, bis- or tris-(2-hydroxyethyl)amine, 2-hydroxy-tert-butylamine, tris(hydroxymethyl)methylamine, N,N-dimethyl-N-(2-hydroxyethyl)amine, tri-(2-hydroxyethyl)amine and N-methyl-D-glucamine. For additional examples of “pharmacologically acceptable salts,” see Berge et al., J. Pharm. Sci. 66:1 (1977).

Compounds disclosed herein can be crystallized and can be provided in a single crystalline form or as a combination of different crystal polymorphs. As such, the compounds can be provided in one or more physical form, such as different crystal forms, crystalline, liquid crystalline or non-crystalline (amorphous) forms. Such different physical forms of the compounds can be prepared using, for example different solvents or different mixtures of solvents for recrystallization. Alternatively, or additionally, different polymorphs can be prepared, for example, by performing recrystallizations at different temperatures and/or by altering cooling rates during recrystallization. The presence of polymorphs can be determined by X-ray crystallography, or in some cases by another spectroscopic technique, such as solid phase NMR spectroscopy, IR spectroscopy, or by differential scanning calorimetry.

The pharmaceutical compositions can be administered to subjects by a variety of mucosal administration modes, including by oral, rectal, intranasal, intrapulmonary, or transdermal delivery, or by topical delivery to other surfaces. Optionally, the compositions can be administered by non-mucosal routes, including by intramuscular, subcutaneous, intravenous, intra-arterial, intra-articular, intraperitoneal, intrathecal, intracerebroventricular, or parenteral routes. In other alternative embodiments, the compound can be administered ex vivo by direct exposure to cells, tissues or organs originating from a subject.

Injectable solutions or suspensions may also be formulated, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The compounds can be administered parenterally, for example, by IV, IM, depo-IM, SC, or depo-SC. When administered parenterally, a therapeutically effective amount of about 0.1 to about 500 mg/day (such as about 1 mg/day to about 100 mg/day, or about 5 mg/day to about 50 mg/day) may be delivered. When a depot formulation is used for injection once a month or once every two weeks, the dose may be about 0.1 mg/day to about 100 mg/day, or a monthly dose of from about 3 mg to about 3000 mg.

The compounds can also be administered sublingually. When given sublingually, the compounds should be given one to four times daily in the amounts described above for IM administration.

The compounds can also be administered intranasally. When given by this route, the appropriate dosage forms are a nasal spray or dry powder. The dosage of the compounds for intranasal administration is the amount described above for IM administration. When administered by nasal aerosol or inhalation, these compositions may be prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents.

The compounds can be administered intrathecally. When given by this route, the appropriate dosage form can be a parenteral dosage form. The dosage of the compounds for intrathecal administration is the amount described above for IM administration.

The compounds can be administered topically. When given by this route, the appropriate dosage form is a cream, ointment, or patch. When administered topically, an illustrative dosage is from about 0.5 mg/day to about 200 mg/day. Because the amount that can be delivered by a patch is limited, two or more patches may be used.

The compounds can be administered rectally by suppository. When administered by suppository, an illustrative therapeutically effective amount may range from about 0.5 mg to about 500 mg. When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

To formulate the pharmaceutical compositions, the compound can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80 or Miglyol 812), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, NJ), Freund's adjuvant, MPL™ (3-O-deacylated monophosphoryl lipid A; Corixa, Hamilton, IN) and IL-12 (Genetics Institute, Cambridge, MA), among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The compound can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively, or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface.

The compound can be combined with the base or vehicle according to a variety of methods, and release of the compound can be by diffusion, disintegration of the vehicle, or associated formation of water channels. In some circumstances, the compound is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the compound can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain embodiments, the compound can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-caprolactone-CO-lactic acid), poly(epsilon.-caprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimcrosal, and the like.

In accordance with the various treatment methods of the disclosure, the compound can be delivered to a subject in a manner consistent with conventional methodologies associated with management of the disorder for which treatment or prevention is sought. In accordance with the disclosure herein, a prophylactically or therapeutically effective amount of the compound and/or other biologically active agent is administered to a subject in need of such treatment for a time and under conditions sufficient to prevent, inhibit, and/or ameliorate a selected disease or condition or one or more symptom(s) thereof.

The administration of the compound of the disclosure can be for either prophylactic or therapeutic purpose. When provided prophylactically, the compound is provided in advance of any symptom. The prophylactic administration of the compound serves to prevent or ameliorate any subsequent disease process. When provided therapeutically, the compound is provided at (or shortly after) the onset of a symptom of disease or infection.

For prophylactic and therapeutic purposes, the compound can be administered to the subject by the oral route or in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, avian, dog, sheep, porcine, feline, non-human primate, and other accepted animal model subjects known in the art. Alternatively, effective dosages can be determined using in vitro models. Using such models, only ordinary calculations and adjustments are required to determine an appropriate concentration and dose to administer a therapeutically effective amount of the compound (for example, amounts that are effective to alleviate one or more symptoms of a targeted disease). In alternative embodiments, an effective amount or effective dose of the compound may simply inhibit or enhance one or more selected biological activities correlated with a disease or condition, as set forth herein, for either therapeutic or diagnostic purposes.

The actual dosage of the compound will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the compound for eliciting the desired activity or biological response in the subject. Dosage regimens can be adjusted to provide an optimum prophylactic or therapeutic response. A therapeutically effective amount is also one in which any toxic or detrimental side effects of the compound and/or other biologically active agent is outweighed in clinical terms by therapeutically beneficial effects. A non-limiting range for a therapeutically effective amount of a compound and/or other biologically active agent within the methods and formulations of the disclosure is about 0.01 mg/kg body weight to about 20 mg/kg body weight, such as about 0.05 mg/kg to about 5 mg/kg body weight, or about 0.2 mg/kg to about 2 mg/kg body weight.

Dosage can be varied by the attending clinician to maintain a desired concentration at a target site (for example, the lungs or systemic circulation). Higher or lower concentrations can be selected based on the mode of delivery, for example, trans-epidermal, rectal, oral, pulmonary, intraosseous, or intranasal delivery versus intravenous or subcutaneous or intramuscular delivery. Dosage can also be adjusted based on the release rate of the administered formulation, for example, of an intrapulmonary spray versus powder, sustained release oral versus injected particulate or transdermal delivery formulations, and so forth.

The compounds disclosed herein may also be co-administered with an additional therapeutic agent. Such agents include, but are not limited to, an antidiabetic agent, a cholesterol-lowering agent, an anti-inflammatory agent, an antimicrobial agent, a matrix metalloprotease inhibitor, a lipoxygenase inhibitor, a cytokine antagonist, an immunosuppressant, an anti-cancer agent, an anti-viral agent, a cytokine, a growth factor, an immunomodulator, a prostaglandin or an anti-vascular hyperproliferation compound.

The instant disclosure also includes kits, packages and multi-container units containing the herein described pharmaceutical compositions, active ingredients, and/or means for administering the same for use in the prevention and treatment of diseases and other conditions in mammalian subjects. Kits for diagnostic use are also provided. In one embodiment, these kits include a container or formulation that contains one or more of the compounds described herein. In one example, this component is formulated in a pharmaceutical preparation for delivery to a subject. The compound is optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the pharmaceutical agent packaged therewith can be used.

Certain embodiments are described below in the following numbered clauses:

1. A compound, or a pharmaceutically acceptable salt or ester thereof, having a structure of:

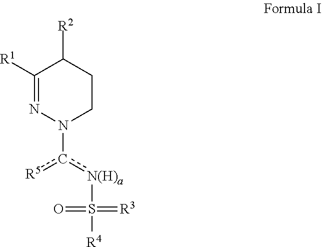

wherein R¹ and R² are each independently aryl, substituted aryl, heteroaryl, or substituted heteroaryl;
R³ is O or NH;
R⁴ is aryl, substituted aryl, heteroaryl, substituted heteroaryl, or N(R¹⁰)(R¹¹), wherein R¹⁰ and R¹¹ are each independently H, optionally-substituted alkyl, optionally-substituted cycloalkyl, halogen, cyano, nitro, hydroxy, optionally-substituted alkoxy, amino, aminocarbonyl, optionally-substituted sulfonyl, optionally-substituted aryl, optionally-substituted heteroaryl, optionally-substituted carboxyl, acyl, optionally-substituted alkenyl, optionally-substituted alkynyl, optionally-substituted phosphonyl, optionally-substituted phosphinyl, aralkyl, or optionally-substituted thiol;
R⁵ is =S, —SR¹², —SeR¹², ═O, —OR¹², —NR¹⁴R¹⁵, —S(O)R¹², —Se(O)R¹², or

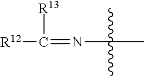

wherein R¹² is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl;
R¹³ is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, allenyl, substituted alkynyl, phosphonyl, substituted phosphonyl, phosphinyl, substituted phosphinyl, boronate, substituted boronate, silyl, substituted silyl, imino, thiol, substituted thiol, selenol, substituted selenol, acyl, or nitrate (ONO₂);
R¹⁴ is H, alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl;
R¹⁵ is H, alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl;
provided that R⁵ is not —N(H)(methyl);
each bond represented by [symbol] is a single or double bond as needed to satisfy valence requirements; and
a is 0 or 1 as needed to satisfy valence requirements.

2. The compound of clause 1, wherein R¹ and R² are each independently phenyl or substituted phenyl.

3. The compound of clause 1, wherein R¹ is halogen-substituted phenyl and R² is phenyl.

4. The compound of clause 1, having a structure of:

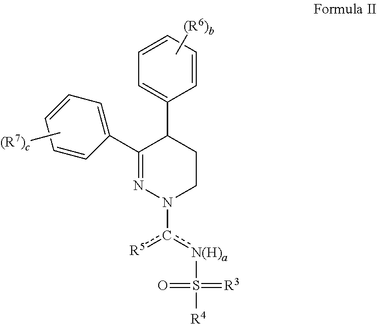

wherein R³, R⁴, R⁵, and subscript a are the same as in formula I;
R⁶ and R⁷ are each independently alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, or substituted thiol;
b is 0 to 5; and
c is 0 to 5.

5. The compound clause 4, wherein b is 0 and c is 1.

6. The compound of clause 4 or 5, wherein R⁷ is halogen.

7. The compound of clause 1, having a structure of:

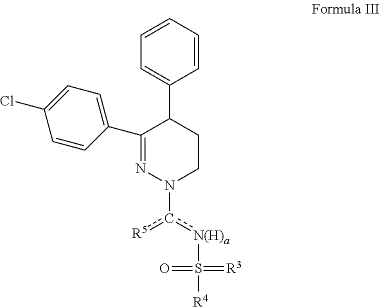

wherein R³, R⁴, R⁵, and subscript a are the same as in formula I.

8. The compound of clause 1, having a structure of:

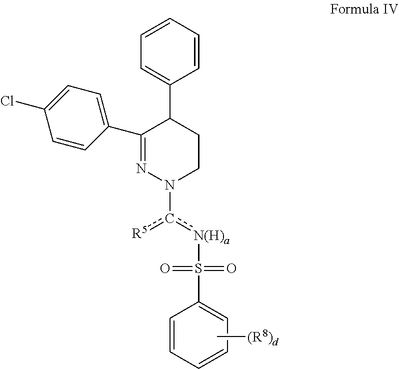

wherein R⁵ and subscript a are the same as in formula I; and
R⁸ is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, or substituted thiol; and d is 0 to 5.

9. The compound of any one of clauses 1 to 8, wherein R³ is O.

10. The compound of any one of clauses 1 to 9, wherein R⁴ is aryl or substituted aryl.

11. The compound of clause 10, wherein R⁴ is phenyl, substituted phenyl, naphthyl, or substituted naphthyl.

12. The compound of clause 10, wherein R⁴ is phenyl, halogen-substituted phenyl, halogenated alkyl-substituted phenyl, alkoxy-substituted phenyl, halogenated alkoxy-substituted phenyl, cyano-substituted phenyl, or naphthyl.

13. The compound of any one of clauses 1 to 12, wherein R⁵ is O.

14. The compound of any one of clauses 1 to 12, wherein R⁵ is S.

15. The compound of any one of clauses 1 to 12, wherein R⁵ is

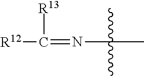

wherein R¹² is amino, substituted amino, acyl, or acetamido.

16. The compound of any one of clauses 1 to 12 or 15, wherein R⁵ is

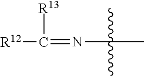

wherein R¹³ is alkyl, alkyl-substituted thiol, alkyl-substituted selenol, acetyl, 2-iminopiperidinyl, propionyl, cinnamoyl, guanidino, Boc-guanidino, or 2-iminopyrrolidinyl.

17. The compound of clause 15 or 16, wherein R¹² is —NH₂.

18. The compound of clause 8, having a structure of:

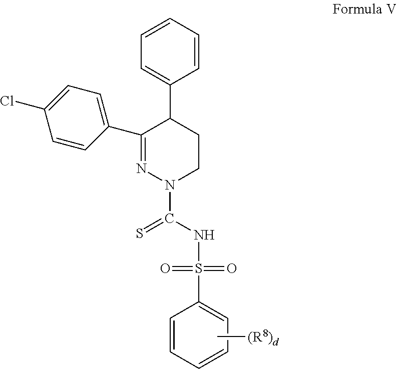

wherein R⁸ and subscript d are the same as in formula IV.

19. The compound of clause 8, having a structure of:

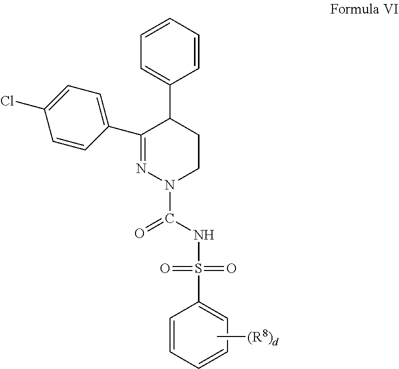

wherein R⁸ and subscript d are the same as in formula IV.

20. The compound of clause 8, having a structure of:

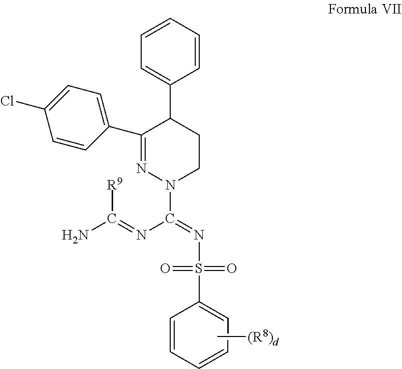

wherein R⁸ and subscript d are the same as in formula IV; and
R⁹ is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, selenol, or substituted selenol.

21. The compound of any one of clauses 8, 18, 19 or 20, wherein R⁸ is halogen, halogenated alkyl, lower alkyl, nitro, naphthyl, halogenated alkoxy, cyano, or alkoxy.

22. The compound of any one of clauses 8, 18, 19, or 20, wherein R⁸ is —Cl, —I, —Br, —F, —CH₃, β-naphthyl, —NO₂, —CF₃, —OCF₃, —CN, or —OCH₃.

23. The compound of any one of clauses 8, 18, 19 or 20, wherein d is 1, R⁸ is in the para position, and is —CF₃ or —OCF₃.

24. A pharmaceutical composition comprising a compound of any one of clauses 1 to 23, and at least one pharmaceutically acceptable additive.

25. A pharmaceutical composition comprising a compound of any one of clauses 1 to 23, wherein the composition is in unit dosage form.

26. A method for reducing food intake and body weight, reversing insulin and leptin resistance, revering hepatic steatosis or improving dyslipidemia in a subject, comprising administering a therapeutically effective amount of a compound of any one of clauses 1 to 23 to a subject in need thereof.

27. A method for treating obesity, diabetes, non-alcoholic fatty liver disease, alcoholic fatty liver disease, insulin resistance, cirrhosis, liver cancer, dyslipidemias that predispose to arteriosclerotic heart disease, diabetic nephropathy, gout, fibrosis, or obesity-induced chronic kidney disease in a subject, comprising administering a therapeutically effective amount of a compound of any one of clauses 1 to 23 to a subject in need thereof.

28. A method for treating a co-morbidity of obesity in a subject, comprising administering a therapeutically effective amount of a compound of any one of clauses 1 to 23 to a subject in need thereof, wherein the co-morbidity is diabetes, Metabolic Syndrome, dementia, heart disease, cancer, hypertension, gallbladder disease, gastrointestinal disorders, menstrual irregularities, degenerative arthritis, venous statis ulcer, pulmonary hypoventilation syndrome, sleep apnea, snoring, coronary artery disease, arterial sclerotic disease, pseudotumor cerebri, accident proneness, increased risks with surgeries, osteoarthritis, high cholesterol, or increased incidence of malignancies of the liver, ovaries, cervix, uterus, breasts, prostrate, or gallbladder.

29. A method of preventing or reversing the deposition of adipose tissue in a subject, or preventing or reversing ectopic deposition of fat in a subject comprising administering a therapeutically effective amount of a compound of any one of clauses 1 to 23 to a subject in need thereof.

30. A method for treating idiopathic pulmonary fibrosis, Hermansky-Pudlak syndrome associated pulmonary fibrosis, radiation induced pulmonary fibrosis, scleroderma associated organ fibrosis, skin fibrosis, kidney fibrosis, liver fibrosis, chronic graft versus host disease (cGvHD) or any combination thereof in a subject, comprising administering a therapeutically effective amount of a compound of any one of clauses 1 to 23 to a subject in need thereof.

### EXAMPLES

### Example 1

### General Method A for the Synthesis of 3-(4-chlorophenyl)-4-phenyl-1,4,5,6-tetrahydropyridazine (6)

To a suspension of NaH (60% dispersion in oil; 2.5 g, 0.0064 mol) in DMSO (50 mL) under nitrogen atmosphere, a solution of the 4 chlorobenzyl phenyl ketones (1) (10 g, 0.0433 mol) in anhydrous DMSO (50 mL) was added dropwise. After the reaction subsides, methyl bromoacetate (2) (4.8 mL, 0.0433 mol) in DMSO (30 mL) was added dropwise and the mixture stirred for 3 hours. The mixture was then acidified by 6N HCl, diluted with water and extracted by dichloromethane. After drying over sodium sulfate, the solvent was evaporated, and the residue purified by flash chromatography to afford the desired product 3 as light-yellow semisolid (2.75 g, 84%).

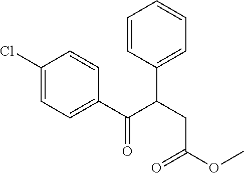 [SMILES: COC(=O)CC(C(=O)c1ccc(Cl)cc1)c1ccccc1]

A mixture of the ester (10 g, 0.034 mol) and hydrazine monohydrate (4) (10 mL, 0.14 mol) in ethanol (50 mL) was refluxed for 24 hours. After cooling, the ethanol was evaporated off and reaction mixture was extracted by ethyl acetate to afford yellowish solid 5 (6.9 g, 85%).

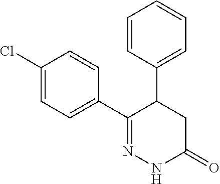 [SMILES: O=C1CC(c2ccccc2)C(c2ccc(Cl)cc2)=NN1]

To LiAIH₄ (476 mg, 12.5 mmol), dry THF (20 ml) was added at 0° C. Compound 5 (1.19 g, 4.18 mmol) was also dissolved in THF (20 ml) and added dropwise to the magnetically stirred LiAIH₄/THF solution at 0° C. Then the mixture was successively stirred for 30 min at reflux temperature (65° C.). After completion of the reaction the mixture was cooled and 2 N NaOH was added carefully followed by the addition of ethyl acetate. The formed mixture was extracted with of ethyl acetate, washed with brine solution and the organic layer was successively dried over sodium sulfate and concentrated in vacuo. Flash chromatography purification afforded the desired compound as yellowish gummy solid 6 (3-(4-chlorophenyl)-4-phenyl-1,4,5,6-tetrahydropyridazine) (1.04 g; 92%) which was solidified after keeping it in the fridge.

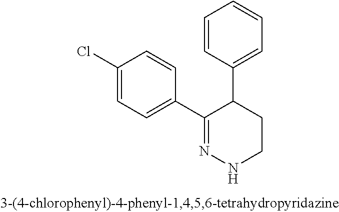 [SMILES: Clc1ccc(C2=NNCCC2c2ccccc2)cc1]

General method for the synthesis of 3-(4-bromophenyl)-4-phenyl-1,4,5,6-tetrahydropyridazine (35) Following the above procedure starting from 6-(4-bromophenyl)-5-phenyl-4,5-dihydropyridazin-3 (2H)-one (1.4 g), 3-(4-bromophenyl)-4-phenyl-1,4,5,6-tetrahydropyridazine (35) isolated as yellow gummy solid (0.8 g, 55%).

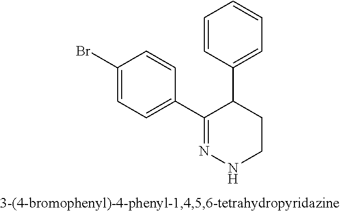 [SMILES: Brc1ccc(C2=NNCCC2c2ccccc2)cc1]

General method for the synthesis of 3-(4-fluorophenyl)-4-phenyl-1,4,5,6-tetrahydropyridazine (36) Following the above procedure starting from 6-(4-fluorophenyl)-5-phenyl-4,5-dihydropyridazin-3 (2H)-one (1.2 g), 3-(4-fluorophenyl)-4-phenyl-1,4,5,6-tetrahydropyridazine (36) isolated as yellow gummy solid (1.0 g, 75%).

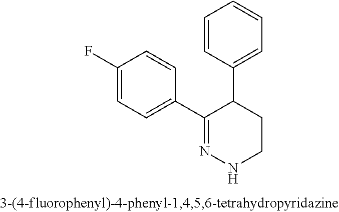 [SMILES: Fc1ccc(C2=NNCCC2c2ccccc2)cc1]

### Example 2

### General Procedure B for the Synthesis of 7a-7m

### Methyl((4-Iodophenyl)sulfonyl)carbamate (7a)

To a solution of 4-iodobenzenesulfonamide (5.0 g, 0.018 mol) stirred at 0° C. in acetonitrile (50 mL), Et₃N (4.5 g, 0.045 mol) was added, followed by the slow addition of methyl chloroformate (2.0 mL, 0.027 mol). The resulting slurry was stirred 16 hours at room temperature. The acetonitrile solution was evaporated, and the residue was dissolved in EtOAc, and a saturated solution of NaHCO₃ was added. The aqueous layer was separated, and ice was added to it followed by acidification with conc. HCl to give precipitate. The precipitate was filtered, washed with water, and dried to afford light brown solid compound H1 (4.8 g, 81%).

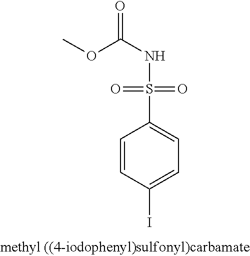

Methyl(Naphthalen-2-ylsulfonyl)carbamate (7b). Refer to general procedure B. White Solid (5.2 g, 83%)

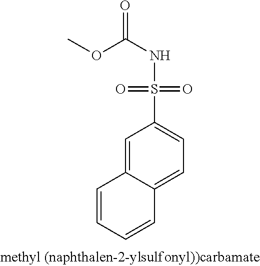 [SMILES: COC(=O)NS(=O)(=O)c1ccc2ccccc2c1]

Methyl ((4-Chlorophenyl)sulfonyl)carbamate (7c). Refer to general procedure B. White Solid (5.2 g, 80%)

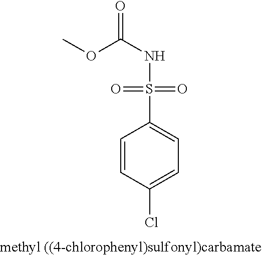

Methyl ((4-Trifluoromethylphenyl)sulfonyl)carbamate (7d). Refer to general procedure B. White Solid (4.9 g, 78%)

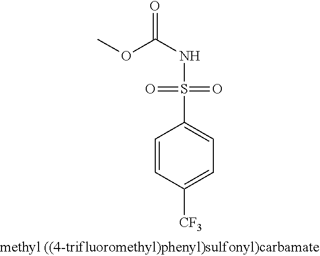 [SMILES: COC(=O)NS(=O)(=O)c1ccc(C(F)(F)F)cc1]

Methyl ((3-Trifluoromethylphenyl)sulfonyl)carbamate (7e). Refer to general procedure B. White Solid (4.7 g, 75%)

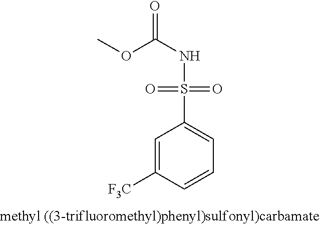 [SMILES: COC(=O)NS(=O)(=O)c1cccc(C(F)(F)F)c1]

Methyl ((4-Trifluoromethoxyphenyl)sulfonyl)carbamate (7f). Refer to general procedure B. White Solid (4.6 g, 74.1%)

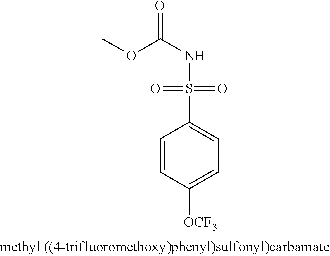 [SMILES: COC(=O)NS(=O)(=O)c1ccc(OC(F)(F)F)cc1]

Methyl ((4-cyanophenyl)sulfonyl)carbamate (7g). Refer to general procedure B. White Solid (5.1 g, 79.6%)

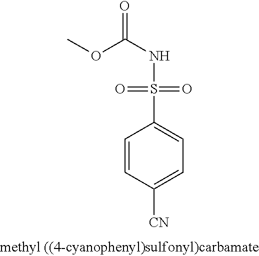 [SMILES: COC(=O)NS(=O)(=O)c1ccc(C#N)cc1]

Methyl (4-methoxyphenyl)sulfonyl)carbamate (7h). Refer to general procedure B. White Solid (2.9 g. 45%)

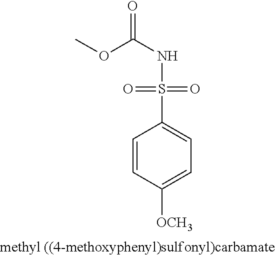 [SMILES: COC(=O)NS(=O)(=O)c1ccc(OC)cc1]

Methyl (phenylsulfonyl)carbamate (71). Refer to general procedure B. White Solid (1.8 g, 26.3%)

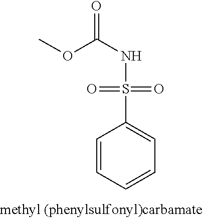

Methyl (pyridin-3-ylsulfonyl)carbamate (7j). Refer to general procedure B. Red Solid (1.3 g, 19%)

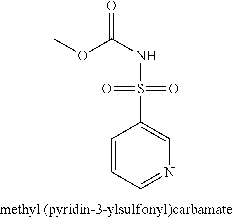

Methyl tosylcarbamate (7k). Refer to general procedure B. White Solid (1.7 g. 25.4%)

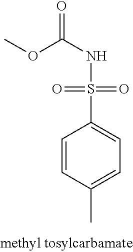

methyl ((2-(trifluoromethyl)phenyl)sulfonyl)carbamate (71), Refer to general procedure B. White Solid (5.8 g, 92%)

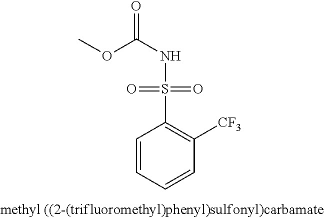 [SMILES: COC(=O)NS(=O)(=O)c1ccccc1C(F)(F)F]

methyl ((4-fluorophenyl)sulfonyl)carbamate (7m). Refer to general procedure B. White Solid (0.46 g, 35%)

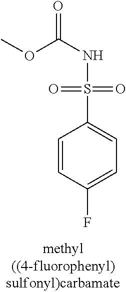

### Example 3

### General Procedure C for the Synthesis of 3-(4-chlorophenyl)-N-((4-chlorophenyl)sulfonyl)-4-phenyl-5,6-dihydropyridazine-1 (4H)-carboxamide (8a)

To a solution of methyl ((4-chlorophenyl)sulfonyl)carbamate (0.47 g 0.0018 mol) in toluene was added 3-(4-chlorophenyl)-4-phenyl-1,4,5,6-tetrahydropyridazine (0.5 g, 0.0018 mol) and the resulting slurry was refluxed for 4 h. After cooling to room temperature, the toluene solution was evaporated, and the slurry was triturated with isopropyl alcohol to obtain white slurry. The solution was filtered and washed with a mixture of cold IPA and hexanes (1:1) to give compound I1 as a white solid (0.7 g, 78%). ¹H NMR (800 MHz, CDCl₃): δ 9.24 (s, 1H), 8.12 (d, J=7.2 Hz, 2H), 7.53 (t, J=8.8 Hz, 4H), 7.31-7.28 (m, 4H), 7.26-7.24 (m, 1H), 7.06 (d, J=7.4 Hz, 2H), 4.18 (d, J=12.0 Hz, 2H), 3.05 (t, J=12.1 Hz, 1H), 2.14-2.06 (m, 2H). LRMS 488.1, HRMS (ESI) m/z: [M+H]+Calcd for (C₂₃ H₂₀ N₃ O₃ S Cl₂) 488.0602; Found 488.0597.

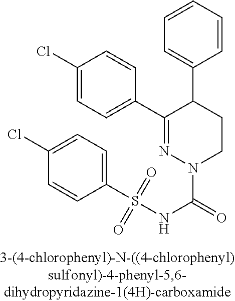 [SMILES: O=C(NS(=O)(=O)c1ccc(Cl)cc1)N1CCC(c2ccccc2)C(c2ccc(Cl)cc2)=N1]

### Synthesis of 3-(4-chlorophenyl)-4-phenyl-N-((4-(trifluoromethyl)phenyl)sulfonyl)-5,6 dihydropyridazine-1 (4H)-carboxamide (8c)

Following above procedure C, 4-trifluoromethylbenzenesulfonamide (0.52 g, 0.0018 mol) gave the carbamate 8c (0.78 g, 81%) as a white solid. 1H-NMR (800 MHz; CDCl₃): δ 9.28 (s, 1H), 8.31 (d, J=8.2 Hz, 2H), 7.82 (d, J=8.2 Hz, 2H), 7.51 (d, J=8.4 Hz, 2H), 7.29 (q, J=8.2 Hz, 4H), 7.24 (d, J=7.2 Hz, 1H), 7.05 (d, J=7.6 Hz, 2H), 4.17 (d, J=16.9 Hz, 2H), 3.04 (td, J=13.1, 3.7 Hz, 1H), 2.13-2.03 (m, 2H). LRMS 522.1, HRMS (ESI) m/z: [M+H]+Calcd for (C₂₄ H₂₀ N₃ O₃ F₃ S Cl) 522.0866; Found 522.0862.

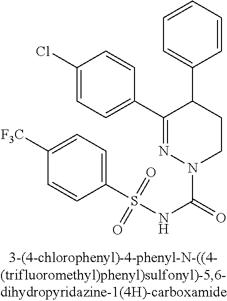 [SMILES: O=C(NS(=O)(=O)c1ccc(C(F)(F)F)cc1)N1CCC(c2ccccc2)C(c2ccc(Cl)cc2)=N1]

### 3-(4-chlorophenyl)-4-phenyl-N-((3-(trifluoromethyl)phenyl)sulfonyl)-5,6-dihydropyridazine-1 (4H)-carboxamide (8b)

Following procedure C, obtained carbamate (0.92 g, 60%) as a white solid. LRMS 522.1, HRMS (ESI) m/z: [M+H]+Calcd for (C24 H20 N3 O3 F3 S Cl) 522.0866; Found 522.0865.

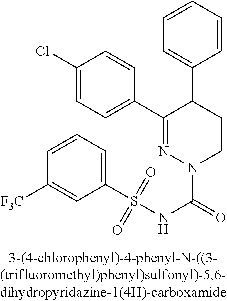 [SMILES: O=C(NS(=O)(=O)c1cccc(C(F)(F)F)c1)N1CCC(c2ccccc2)C(c2ccc(Cl)cc2)=N1]

### 3-(4-chlorophenyl)-4-phenyl-N-((4-(trifluoromethoxy)phenyl)sulfonyl)-5,6-dihydropyridazine-1 (4H)-carboxamide (8d)

Following procedure C, obtained carbamate (0.63 g, 61%) as a white solid. LRMS 538.1, HRMS (ESI) m/z: [M+H]+Calcd for (C24 H20 N3 O4 F3 S Cl) 538.0815; Found 538.0819.

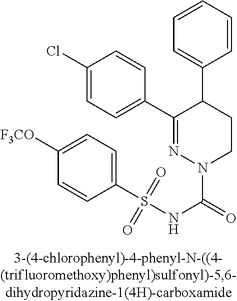 [SMILES: O=C(NS(=O)(=O)c1ccc(OC(F)(F)F)cc1)N1CCC(c2ccccc2)C(c2ccc(Cl)cc2)=N1]

### 3-(4-chlorophenyl)-N-((4-cyanophenyl)sulfonyl)-4-phenyl-5,6-dihydropyridazine-1 (4H)-carboxamide (8e)

Following procedure C, obtained carbamate (1.2 g, 65%) as a light brown solid. LRMS 479.1, HRMS (ESI) m/z: [M+H]+Calcd for (C24 H20 N4 O3 S Cl) 479.0945; Found 479.0939.

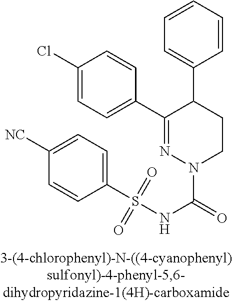 [SMILES: N#Cc1ccc(S(=O)(=O)NC(=O)N2CCC(c3ccccc3)C(c3ccc(Cl)cc3)=N2)cc1]

### 3-(4-chlorophenyl)-N-(naphthalen-2-ylsulfonyl)-4-phenyl-5,6-dihydropyridazine-1 (4H)-carboxamide (8f)

Following procedure, obtained carbamate (1.5 g, 80%) as a pale-yellow solid. LRMS 504.1, HRMS (ESI) m/z: [M+H]+Calcd for (C27 H23 N3 O3 S Cl) 504.1149; Found 504.1146.

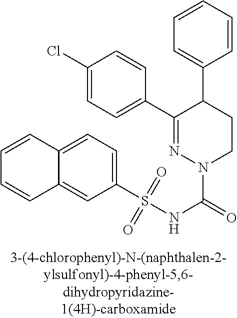 [SMILES: O=C(NS(=O)(=O)c1ccc2ccccc2c1)N1CCC(c2ccccc2)C(c2ccc(Cl)cc2)=N1]

### 3-(4-chlorophenyl)-N-((4-methoxyphenyl)sulfonyl)-4-phenyl-5,6-dihydropyridazine-1 (4H)-carboxamide (8g)

Following procedure C, obtained carbamate (1.0 g, 75%) as a white solid. LRMS 484.1, HRMS (ESI) m/z: [M+H]+Calcd for (C24 H23 N3 O4 S Cl) 484.1098; Found 484.1091.

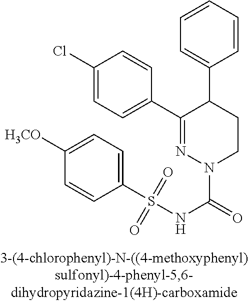 [SMILES: COc1ccc(S(=O)(=O)NC(=O)N2CCC(c3ccccc3)C(c3ccc(Cl)cc3)=N2)cc1]

### 3-(4-chlorophenyl)-4-phenyl-N-(phenylsulfonyl)-5,6-dihydropyridazine-1 (4H)-carboxamide (8h)

Following procedure C, obtained carbamate (0.33 g, 85%) as a white solid. LRMS 454.1, HRMS (ESI) m/z: [M+H]+Calcd for (C23 H21 N3 O3 S Cl) 454.0992; Found 454.0988.

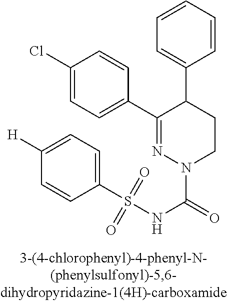 [SMILES: O=C(NS(=O)(=O)c1ccccc1)N1CCC(c2ccccc2)C(c2ccc(Cl)cc2)=N1]

### 3-(4-chlorophenyl)-N-((4-iodophenyl)sulfonyl)-4-phenyl-5,6-dihydropyridazine-1 (4H)-carboxamide (8i)

Following procedure C, obtained carbamate (0.287 g, 34%) as a white solid. LRMS 580.0, HRMS (ESI) m/z: [M+H]+Calcd for (C23 H20 N3 O3 S Cl I) 579.9959; Found 579.9947.

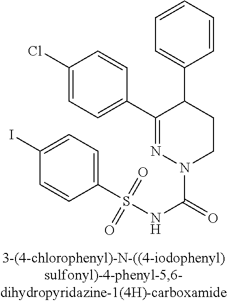 [SMILES: O=C(NS(=O)(=O)c1ccc(I)cc1)N1CCC(c2ccccc2)C(c2ccc(Cl)cc2)=N1]

### 3-(4-chlorophenyl)-N-((4-fluorophenyl)sulfonyl)-4-phenyl-5,6-dihydropyridazine-1 (4H)-carboxamide (8j)

Following procedure C, obtained carbamate (0.73 g, 90%) as a white solid. LRMS 472.1, HRMS (ESI) m/z: [M+H]+Calcd for (C23 H20 N3 O3 F S Cl) 472.0898; Found 472.0891.

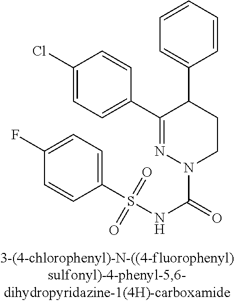 [SMILES: O=C(NS(=O)(=O)c1ccc(F)cc1)N1CCC(c2ccccc2)C(c2ccc(Cl)cc2)=N1]

### 3-(4-chlorophenyl)-4-phenyl-N-((2-(trifluoromethyl)phenyl)sulfonyl)-5,6-dihydropyridazine-1 (4H)-carboxamide (8k)

Following procedure, obtained carbamate (0.655 g, 85%) as a white solid. LRMS 522.1, HRMS (ESI) m/z: [M+H]+Calcd for (C24 H20 N3 O3 F3 S Cl) 522.0866; Found 522.0863.

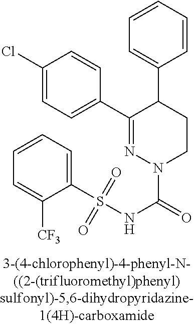 [SMILES: O=C(NS(=O)(=O)c1ccccc1C(F)(F)F)N1CCC(c2ccccc2)C(c2ccc(Cl)cc2)=N1]

### 3-(4-chlorophenyl)-4-phenyl-N-tosyl-5,6-dihydropyridazine-1 (4H)-carboxamide (81)

Following procedure, obtained carbamate (0.601 g, 86.8%) as a white solid. LRMS 468.1, HRMS (ESI) m/z: [M+H]+Calcd for (C24 H23 N3 O3 S Cl) 468.1149; Found 468.1141.

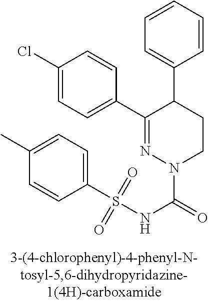 [SMILES: Cc1ccc(S(=O)(=O)NC(=O)N2CCC(c3ccccc3)C(c3ccc(Cl)cc3)=N2)cc1]

### 3-(4-fluorophenyl)-4-phenyl-N-((4-(trifluoromethyl)phenyl)sulfonyl)-5,6-dihydropyridazine-1 (4H)-carboxamide (8m)

Following procedure, obtained carbamate (0.35 g, 70%) as a white solid. LRMS 506.1.

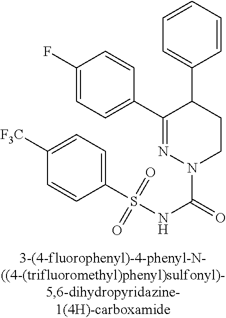 [SMILES: O=C(NS(=O)(=O)c1ccc(C(F)(F)F)cc1)N1CCC(c2ccccc2)C(c2ccc(F)cc2)=N1]

### 3-(4-bromophenyl)-4-phenyl-N-((4-(trifluoromethyl)phenyl)sulfonyl)-5,6-dihydropyridazine-1 (4H)-carboxamide (8n)

Following procedure, obtained carbamate (1.3 g, 90%) as a white solid. LRMS 566.1.

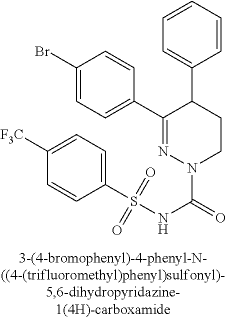 [SMILES: O=C(NS(=O)(=O)c1ccc(C(F)(F)F)cc1)N1CCC(c2ccccc2)C(c2ccc(Br)cc2)=N1]

### General Procedure D for the Synthesis of (Z)—N—((E)-1-aminoethylidene)-3-(4-chlorophenyl)-N′-((4-chlorophenyl)sulfonyl)-4-phenyl-5,6-dihydropyridazine-1 (4H)-carboximidamide (10a)

To a mixture of compound, 8a (200 mg, 0.41 mmol) in toluene (2 mL), POCl₃ (58 μL, 0.615 mmol), was added, followed by the addition of DIPEA (144 μL, 0.82 mmol) and the mixture was refluxed for 3-4 h. The reaction mixture was then cooled, and toluene was evaporated in vacuo. The crude imidoyl chloride compound was dissolved in dichloromethane (1 mL), and to it was added a premixed mixture of acetamidine hydrochloride (175 mg, 1.84 mmol) in methanol/dichloromethane (1:4, 2 mL) and Et₃N (344 μL, 2.46 mmol) at room temperature dropwise. The mixture was allowed to warm up to room temperature and stirred for 18 hrs. The reaction mixture was extracted into dichloromethane, washed with water, and purified by flash chromatography using hexanes/EtOAC (4:6) followed by trituration with IPA/hexane to afford compound (Z)—N—((E)-1-aminoethylidene)-3-(4-chlorophenyl)-N′-((4-chlorophenyl)sulfonyl)-4-phenyl-5,6-dihydropyridazine-1 (4H)-carboximidamide (61 mg, 23%) as white solid. ¹H-NMR (800 MHZ; CDCl₃): δ 7.89 (d, J=8.0 Hz, 2H), 7.53 (d, J=8.1 Hz, 2H), 7.40 (d, J=7.9 Hz, 2H), 7.29 (t, J=7.4 Hz, 2H), 7.24 (d, J=7.0 Hz, 1H), 7.19 (d, J=8.2 Hz, 2H), 7.06 (d, J=7.5 Hz, 2H), 4.50 (d, J=13.0 Hz, 1H), 4.15 (d, J=3.8 Hz, 1H), 3.22 (td, J=13.5, 3.5 Hz, 1H), 2.18 (tt, J=13.4, 5.0 Hz, 1H), 2.08 (dd, J=13.5, 2.0 Hz, 1H), 2.02 (s, 3H). 13C NMR (201 MHz, CDCl₃) & 160.58, 159.60, 148.50, 141.67, 140.48, 137.71, 135.33, 134.79, 129.19, 128.84, 128.67, 128.42, 127.82, 127.45, 127.17, 77.17, 77.01, 76.85, 37.93, 37.73, 26.32, 20.75. HRMS [M+H]⁺: found m/z 528.1037, calcd 528.1028.

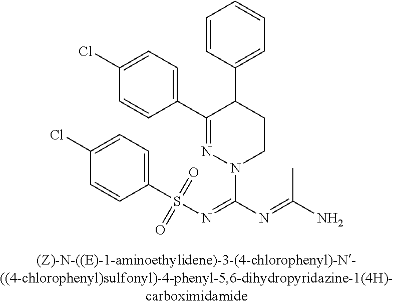 [SMILES: C/C(N)=N\C(=N\S(=O)(=O)c1ccc(Cl)cc1)N1CCC(c2ccccc2)C(c2ccc(Cl)cc2)=N1]

### General Procedure E for the synthesis of methyl (Z)—N′—((Z)-(3-(4-chlorophenyl)-4-phenyl-5,6-dihydropyridazin-1 (4H)-yl)(((4-chlorophenyl)sulfonyl)imino)methyl)carbamimidothioate (11a)

To a mixture of compound, 8a (200 mg, 0.41 mmol) in toluene (2 mL), POCl₃ (58 μL, 0.615 mmol), was added, followed by the addition of DIPEA (144 μL, 0.82 mmol) and the mixture was refluxed for 3-4 h. The reaction mixture was then cooled, and toluene was evaporated in vacuo. The crude imidoyl chloride compound was dissolved in dichloromethane (1 mL), and to it was added a premixed mixture of methyl carbamimidothioate iodide (402 mg, 1.84 mmol) in methanol/dichloromethane (1:8, 2 mL) and Et₃N (344 μL, 2.46 mmol) at room temperature dropwise. The mixture was allowed to warm up to room temperature and stirred for 18 hrs. The reaction mixture was extracted into dichloromethane, washed with water, and purified by flash chromatography using hexanes/EtOAC (1:1) followed by trituration with IPA/hexane to afford compound (Z)—N′—((Z)-(3-(4-chlorophenyl)-4-phenyl-5,6-dihydropyridazin-1 (4H)-yl)(((4-chlorophenyl)sulfonyl)imino)methyl)carbamimidoselenoate (86 mg, 38%) as white solid.

¹H-NMR (800 MHZ; CDCl₃): δ 7.90 (d, J=7.7 Hz, 2H), 7.57 (s, 2H), 7.41 (d, J=7.7 Hz, 2H), 7.30 (t, J=7.1 Hz, 2H), 7.24 (d, J=7.5 Hz, 1H), 7.20 (d, J=8.0 Hz, 2H), 7.08 (d, J=7.4 Hz, 2H), 5.28 (d, J=36.6 Hz, 1H), 4.48 (d, J=13.4 Hz, 1H), 4.16 (s, 1H), 3.25 (t, J=11.5 Hz, 1H), 2.29 (s, 3H), 2.24-2.21 (m, 1H), 2.08 (dd, J=12.7, 1.3 Hz, 1H). 13C NMR (201 MHz, CDCl₃) δ 159.96, 159.52, 148.79, 140.47, 137.70, 135.38, 134.77, 129.18, 128.74, 128.62, 128.47, 127.88, 127.43, 77.17, 77.01, 76.85, 38.17, 37.76, 29.72, 26.34, 14.26. HRMS [M+H]⁺: found m/z 560.0751, calcd 560.0748.

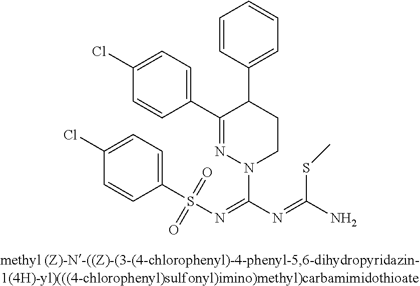 [SMILES: CS/C(N)=N\C(=N\S(=O)(=O)c1ccc(Cl)cc1)N1CCC(c2ccccc2)C(c2ccc(Cl)cc2)=N1]

### General Procedure F for the Synthesis of methyl (Z)—N′—((Z)-(3-(4-chlorophenyl)-4-phenyl-5,6-dihydropyridazin-1 (4H)-yl)(((4-chlorophenyl)sulfonyl)imino)methyl)carbamimidoselenoate (12a)

To a mixture of compound, 8a (200 mg, 0.41 mmol) in toluene (2 mL), POCl₃ (58 μL, 0.615 mmol), was added, followed by the addition of DIPEA (144 μL, 0.82 mmol) and the mixture was refluxed for 3-4 h. The reaction mixture was then cooled, and toluene was evaporated in vacuo. The crude imidoyl chloride compound was dissolved in dichloromethane (1 mL), and to it was added a premixed mixture of methyl carbamimidoselenoate iodide (402 mg, 1.84 mmol) in methanol/dichloromethane (1:8, 2 mL) and Et₃N (344 μL, 2.46 mmol) at room temperature dropwise. The mixture was allowed to warm up to room temperature and stirred for 18 hrs. The reaction mixture was extracted into dichloromethane, washed with water, and purified by flash chromatography using hexanes/EtOAC (1:1) followed by trituration with IPA/hexane to afford compound methyl (Z)—N′—((Z)-(3-(4-chlorophenyl)-4-phenyl-5,6-dihydropyridazin-1 (4H)-yl)(((4-chlorophenyl)sulfonyl)imino)methyl)carbamimidoselenoate (90 mg, 36%) as white solid. ¹H-NMR (800 MHZ; CDCl₃): δ 7.91 (d, J=7.5 Hz, 2H), 7.58 (s, 2H), 7.41 (d, J=7.9 Hz, 2H), 7.28 (t, J=6.4 Hz, 2H), 7.24 (t, J=7.1 Hz, 1H), 7.20 (d, J=7.8 Hz, 2H), 7.08 (d, J=6.3 Hz, 2H), 5.65 (s, 2H), 4.46 (d, J=12.7 Hz, 1H), 4.15 (s, 1H), 3.24 (t, J=12.7 Hz, 1H), 2.23 (s, 3H), 2.21-2.06 (m, 2H). HRMS [M+H]⁺: found m/z 608.0193, calcd 608.0193.

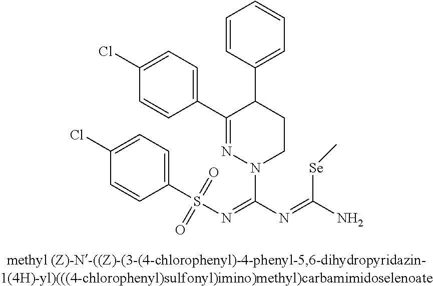 [SMILES: C[Se]/C(N)=N\C(=N\S(=O)(=O)c1ccc(Cl)cc1)N1CCC(c2ccccc2)C(c2ccc(Cl)cc2)=N1]

### Synthesis of (Z)—N—((E)-1-aminoethylidene)-3-(4-chlorophenyl)-4-phenyl-N′-((4-(trifluoromethyl)phenyl)sulfonyl)-5,6-dihydropyridazine-1 (4H)-carboximidamide (10b)

Following general procedure D, starting material (200 mg) gave final compound (57 mg, 26.5%) as a white solid. 1H-NMR (800 MHz; CDCl3): δ 8.08 (d, J=8.0 Hz, 2H), 7.70 (d, J=8.1 Hz, 2H), 7.53 (d, J=8.0 Hz, 2H), 7.29 (t, J=7.6 Hz, 2H), 7.24 (d, J=7.9 Hz, 1H), 7.19 (d, J=8.1 Hz, 2H), 7.06 (d, J=7.4 Hz, 2H), 5.32 (s, 2H), 4.51 (d, J=11.9 Hz, 1H), 4.17 (d, J=3.7 Hz, 1H), 3.24 (td, J=13.3, 3.2 Hz, 1H), 2.21-2.17 (m, 1H), 2.09 (dd, J=13.6, 1.7 Hz, 1H), 2.03 (s, 3H). LRMS 562.1, HRMS (EST) m/z: [M+H]+Calcd for (C26 H24 N5 O2 F3 S Cl) 562.1291; Found 562.1288.

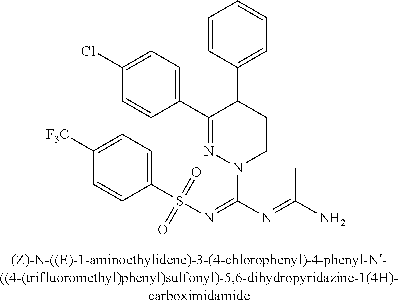 [SMILES: C/C(N)=N\C(=N\S(=O)(=O)c1ccc(C(F)(F)F)cc1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)(((4-(trifluoromethyl)phenyl)sulfonyl)imino)methyl)carbamimidothioate (11b)

Following general procedure E, starting material (200 mg) gave final compound (143 mg, 62%) as a white solid. 1H-NMR (800 MHz; CDCl3): δ 8.09 (d, J=8.1 Hz, 2H), 7.71 (d, J=8.0 Hz, 2H), 7.56 (s, 2H), 7.29 (t, J=7.4 Hz, 2H), 7.24 (d, J=6.5 Hz, 1H), 7.20 (d, J=8.7 Hz, 2H), 7.08 (d, J=7.2 Hz, 2H), 5.30 (s, 1H), 4.49 (d, J=13.4 Hz, 1H), 4.17 (d, J=3.4 Hz, 1H), 3.26 (t, J=11.7 Hz, 1H), 2.24 (s, 3H), 2.09 (d, J=13.4 Hz, 2H). LRMS 594.1, HRMS (ESI) m/z: [M+H]+Calcd for (C26 H24 N5 O2 F3 S2 Cl) 594.1012; Found 594.1013.

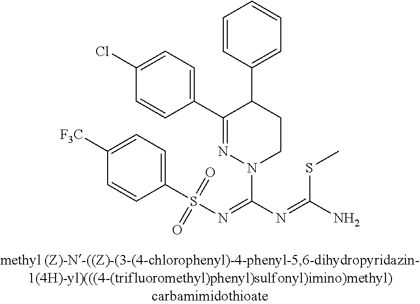 [SMILES: CS/C(N)=N\C(=N\S(=O)(=O)c1ccc(C(F)(F)F)cc1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)(((4-(trifluoromethyl)phenyl)sulfonyl)imino)methyl)carbamimidoselenoate (12b)

Following general procedure F, starting material (200 mg) gave final compound (127 mg, 51.7%) as a white solid. 1H-NMR (800 MHz; CDCl3): δ 8.10 (d, J=6.2 Hz, 2H), 7.71 (d, J=7.4 Hz, 2H), 7.57 (s, 2H), 7.28 (d, J=6.3 Hz, 2H), 7.24 (s, 1H), 7.20 (d, J=6.1 Hz, 2H), 7.08 (d, J=5.6 Hz, 2H), 5.65 (s, 2H), 4.48 (d, J=12.6 Hz, 1H), 4.16 (s, 1H), 3.25 (t, J=13.0 Hz, 1H), 2.20 (d, J=24.3 Hz, 4H), 2.08 (d, J=13.1 Hz, 1H). LRMS 642.1, HRMS (ESI) m/z: [M+H]+Calcd for (C26 H24 N5 O2 F3 S Cl Se) 642.0457; Found 642.0458.

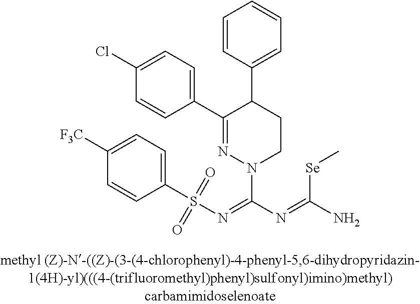 [SMILES: C[Se]/C(N)=N\C(=N\S(=O)(=O)c1ccc(C(F)(F)F)cc1)N1CCC(c2ccccc2)C(c2ccc(Cl)cc2)=N1]

### Synthesis of (Z)—N—((E)-1-aminoethylidene)-3-(4-chlorophenyl)-4-phenyl-N′-((3-(trifluoromethyl)phenyl)sulfonyl)-5,6-dihydropyridazine-1 (4H)-carboximidamide (10c)

Following general procedure D, starting material (300 mg) gave final compound (86 mg, 26.6%) as a white solid. 1H-NMR (800 MHz; CDCl3): δ 8.25 (s, 1H), 8.14 (d, J=7.9 Hz, 1H), 7.74 (d, J=7.6 Hz, 1H), 7.59-7.52 (m, 3H), 7.30 (t, J=7.4 Hz, 2H), 7.25 (d, J=7.5 Hz, 1H), 7.19 (d, J=8.8 Hz, 2H), 7.06 (d, J=7.3 Hz, 2H), 5.17 (s, 2H), 4.52 (d, J=11.5 Hz, 1H), 4.16 (d, J=4.1 Hz, 1H), 3.24 (td, J=13.5, 3.8 Hz, 1H), 2.19 (ddq, J=13.6, 9.0, 4.6 Hz, 1H), 2.10 (dd, J=13.6, 1.9 Hz, 1H), 2.04 (s, 3H). LRMS 562.1, HRMS (ESI) m/z: [M+H]+Calcd for (C26 H24 N5 O2 F3 S Cl) 562.1291; Found 562.1291.

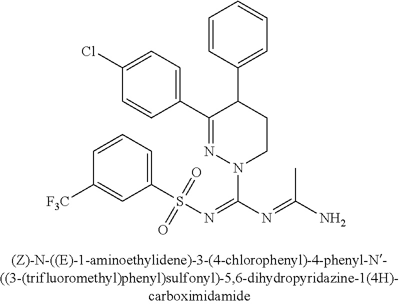 [SMILES: C/C(N)=N\C(=N\S(=O)(=O)c1cccc(C(F)(F)F)c1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)(((3-(trifluoromethyl)phenyl)sulfonyl)imino)methyl)carbamimidothioate (11c)

Following general procedure E, starting material (200 mg) gave final compound (150 mg, 65.9%) as a white solid. 1H-NMR (800 MHz; CDCl3): δ 8.26 (s, 1H), 8.15 (d, J=0.1 Hz, 1H), 7.75 (d, J=7.6 Hz, 1H), 7.58 (dd, J=19.1, 11.3 Hz, 3H), 7.30 (t, J=7.4 Hz, 2H), 7.25 (d, J=7.5 Hz, 1H), 7.20 (d, J=8.7 Hz, 2H), 7.09 (d, J=7.4 Hz, 2H), 5.28 (d, J=42.4 Hz, 2H), 4.50 (d, J=13.2 Hz, 1H), 4.17 (s, 1H), 3.27 (t, J=12.1 Hz, 1H), 2.26 (s, 3H), 2.09 (d, J=13.5 Hz, 2H). LRMS 594.1, HRMS (ESI) m/z: [M+H]+Calcd for (C26 H24 N5 O2 F3 S2 Cl) 594.1012; Found 594.1011.

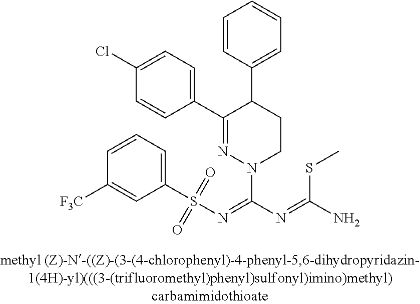 [SMILES: CS/C(N)=N\C(=N\S(=O)(=O)c1cccc(C(F)(F)F)c1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)(((3-(trifluoromethyl)phenyl)sulfonyl)imino)methyl)carbamimidoselenoate (12c)

Following general procedure F, starting material (200 mg) gave final compound (90 mg, 36.6%) as a yellowish white solid. 1H-NMR (800 MHz; CDCl₃): δ 8.26 (s, 1H), 8.16 (d, J=5.7 Hz, 1H), 7.74 (d, J=3.9 Hz, 1H), 7.58 (d, J=7.1 Hz, 3H), 7.29 (d, J=6.0 Hz, 2H), 7.26 (s, 1H), 7.20 (t, J=4.4 Hz, 2H), 7.09 (s, 2H), 5.55 (s, 1H), 4.48 (d, J=13.0 Hz, 1H), 4.17 (s, 1H), 3.26 (t, J=14.1 Hz, 1H), 2.22 (s, 4H), 2.09 (d, J=13.5 Hz, 1H). LRMS 642.1, HRMS (ESI) m/z: [M+H]+Calcd for (C26 H24 N5 O2 F3 S Cl Se) 642.0457; Found 642.0460.

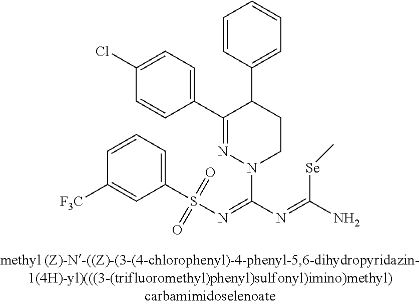 [SMILES: C[Se]/C(N)=N\C(=N\S(=O)(=O)c1cccc(C(F)(F)F)c1)N1CCC(c2ccccc2)C(c2ccc(Cl)cc2)=N1]

### Synthesis of (Z)—N—((E)-1-aminoethylidene)-3-(4-chlorophenyl)-4-phenyl-N′-((4-(trifluoromethoxy)phenyl)sulfonyl)-5,6-dihydropyridazine-1 (4H)-carboximidamide (10d)

Following general procedure D, starting material (300 mg) gave final compound (114 mg, 35.3%) as a white solid. 1H-NMR (800 MHz; CDCl3): δ 8.00 (d, J=7.9 Hz, 2H), 7.54 (d, J=8.0 Hz, 2H), 7.29 (dd, J=16.8, 9.8 Hz, 4H), 7.25 (d, J=7.0 Hz, 1H), 7.20 (d, J=7.4 Hz, 2H), 7.06 (d, J=7.5 Hz, 2H), 5.26 (s, 2H), 4.51 (d, J=12.4 Hz, 1H), 4.16 (d, J=3.1 Hz, 1H), 3.23 (td, J=13.5, 3.1 Hz, 1H), 2.21-2.16 (m, 1H), 2.09 (dd, J=13.4, 1.0 Hz, 1H), 2.02 (s, 3H). LRMS 578.1, HRMS (ESI) m/z: [M+H]+Calcd for (C26 H24 N5 O3 S F3 Cl) 578.1240; Found 578.1247.

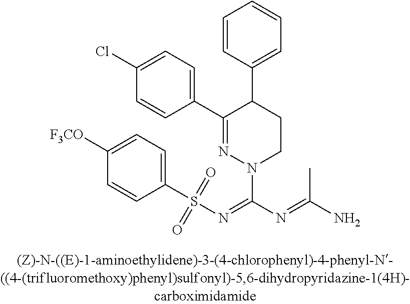 [SMILES: C/C(N)=N\C(=N\S(=O)(=O)c1ccc(OC(F)(F)F)cc1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)(((4-(trifluoromethoxy)phenyl)sulfonyl)imino)methyl)carbamimidothioate (11d)

Following general procedure E, starting material (150 mg) gave final compound (91 mg, 53.5%) as a white solid. 1H-NMR (800 MHz; CDCl3): δ 8.01 (d, J=8.5 Hz, 2H), 7.56 (s, 2H), 7.29 (dt, J=14.0, 7.3 Hz, 4H), 7.24 (d, J=7.1 Hz, 1H), 7.20 (d, J=8.5 Hz, 2H), 7.09 (d, J=7.4 Hz, 2H), 5.24 (s, 2H), 4.49 (d, J=13.5 Hz, 1H), 4.16 (d, J=3.3 Hz, 1H), 3.26 (t, J=12.8 Hz, 1H), 2.25 (s, 3H), 2.10-2.04 (m, 2H). LRMS 610.1, HRMS (ESI) m/z: [M+H]+Calcd for (C26 H24 N5 O3 F3 S2 Cl) 610.0961; Found 610.0962.

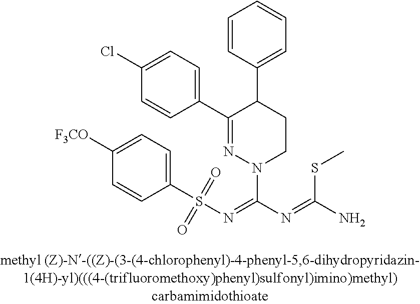 [SMILES: CS/C(N)=N\C(=N\S(=O)(=O)c1ccc(OC(F)(F)F)cc1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)(((4-(trifluoromethoxy)phenyl)sulfonyl)imino)methyl)carbamimidoselenoate (12d)

Following general procedure F, starting material (150 mg) gave final compound (95 mg, 51.8%) as a pale yellow solid. 1H-NMR (800 MHz; CDCl3): δ 8.02 (d, J=6.7 Hz, 2H), 8.02 (d, J=6.7 Hz, 2H), 7.58 (s, 2H), 7.58 (s, 2H), 7.28 (dd, J=15.3, 7.0 Hz, 5H), 7.28 (dd, J=15.3, 7.0 Hz, 5H), 7.20 (d, J=6.8 Hz, 2H), 7.20 (d, J=6.8 Hz, 2H), 7.09 (d, J=6.4 Hz, 2H), 7.09 (d, J=6.4 Hz, 2H), 5.56 (s, 2H), 5.56 (s, 2H), 4.48 (d, J=12.5 Hz, 1H), 4.48 (d, J=12.5 Hz, 1H), 4.17 (s, 1H), 4.17 (s, 1H), 3.25 (t, J=13.0 Hz, 1H), 3.25 (t, J=13.0 Hz, 1H), 2.21 (s, 4H), 2.21 (s, 4H), 2.08 (d, J=13.1 Hz, 1H), 2.09-2.04 (m, 1H). LRMS 658.0, HRMS (ESI) m/z: [M+H]+Calcd for (C26 H24 N5 O3 F3 S Cl Se) 658.0406; Found 658.0410.

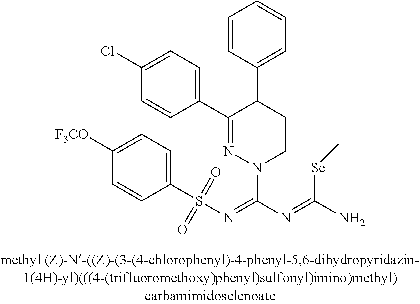 [SMILES: C[Se]/C(N)=N\C(=N\S(=O)(=O)c1ccc(OC(F)(F)F)cc1)N1CCC(c2ccccc2)C(c2ccc(Cl)cc2)=N1]

### Synthesis of (Z)—N—((E)-1-aminoethylidene)-3-(4-chlorophenyl)-N′-((4-cyanophenyl)sulfonyl)-4-phenyl-5,6-dihydropyridazine-1 (4H)-carboximidamide (10e)

Following general procedure D, starting material (300 mg) gave final compound (80 mg, 24.6%) as a pale yellow solid. ¹H-NMR (800 MHz; CDCl₃): δ 8.06 (d, J=7.3 Hz, 2H), 7.73 (d, J=7.0 Hz, 2H), 7.53 (d, J=7.3 Hz, 2H), 7.30 (t, J=6.9 Hz, 2H), 7.25 (s, 1H), 7.20-7.19 (m, 2H), 7.06 (d, J=7.1 Hz, 2H), 5.31 (s, 2H), 4.50 (d, J=12.9 Hz, 1H), 4.17 (d, J=0.6 Hz, 1H), 3.23 (t, J=13.3 Hz, 1H), 2.21-2.17 (m, 1H), 2.10 (d, J=13.5 Hz, 1H), 2.04 (s, 3H). LRMS 519.1, HRMS (ESI) m/z: [M+H]+Calcd for (C26 H24 N6 O2 S Cl) 519.1370; Found 519.1369.

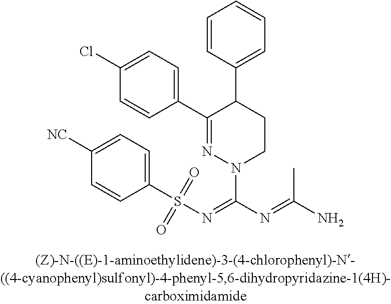 [SMILES: C/C(N)=N\C(=N\S(=O)(=O)c1ccc(C#N)cc1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)(((4-cyanophenyl)sulfonyl)imino)methyl)carbamimidothioate (11e)

Following general procedure E, starting material (150 mg) gave final compound (72 mg, 38.4%) as a white solid. 1H-NMR (800 MHz; CDCl3): δ 8.07 (d, J=7.8 Hz, 2H), 7.74 (d, J=7.9 Hz, 2H), 7.56 (s, 2H), 7.30 (t, J=7.5 Hz, 2H), 7.27 (s, 1H), 7.21 (d, J=8.1 Hz, 2H), 7.08 (d, J=7.0 Hz, 2H), 5.26 (d, J=60.9 Hz, 2H), 4.48 (d, J=13.4 Hz, 1H), 4.18 (d, J=1.5 Hz, 1H), 3.26 (t, J=12.0 Hz, 1H), 2.26 (d, J=28.0 Hz, 3H), 2.24-2.04 (m, 2H). LRMS 551.1, HRMS (ESI) m/z: [M+H]+Calcd for (C26 H24 N6 O2 S2 Cl) 551.1091; Found 551.1091.

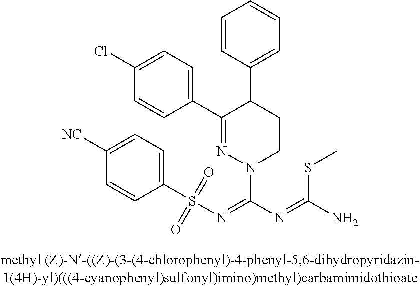 [SMILES: CS/C(N)=N\C(=N\S(=O)(=O)c1ccc(C#N)cc1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)(((4-cyanophenyl)sulfonyl)imino)methyl)carbamimidoselenoate (12e)

Following general procedure F, starting material (150 mg) gave final compound (70 mg, 37.3%) as a pale yellow solid. 1H-NMR (800 MHz; CDCl3): δ 8.08 (d, J=5.5 Hz, 2H), 7.74 (d, J=6.6 Hz, 2H), 7.57 (s, 2H), 7.29 (d, J=5.5 Hz, 2H), 7.26-7.23 (m, 1H), 7.20 (d, J=7.0 Hz, 2H), 7.09 (d, J=3.5 Hz, 2H), 5.65 (s, 2H), 4.46 (d, J=14.5 Hz, 1H), 3.25 (d, J=11.0 Hz, 1H), 2.22 (s, 4H), 2.09-2.04 (m, 1H). LRMS 599.0, HRMS (ESI) m/z: [M+H]+Calcd for (C26 H24 N6 O2 S Cl Se) 599.0535; Found 599.0544.

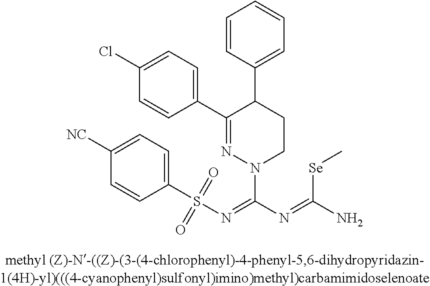 [SMILES: C[Se]/C(N)=N\C(=N\S(=O)(=O)c1ccc(C#N)cc1)N1CCC(c2ccccc2)C(c2ccc(Cl)cc2)=N1]

### Synthesis of (Z)—N—((E)-1-aminoethylidene)-3-(4-chlorophenyl)-4-phenyl-N′-(phenylsulfonyl)-5,6-dihydropyridazine-1 (4H)-carboximidamide (10g)

Following general procedure D, starting material (300 mg) gave final compound (38 mg, 11.7%) as a white solid. LRMS 494.1, HRMS (EST) m/z: [M+H]+Calcd for (C25 H25 N5 O2 S Cl) 494.1417; Found 494.1413.

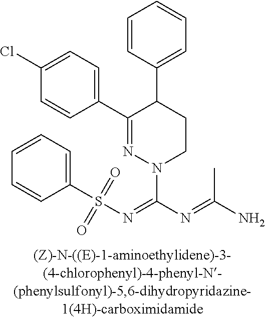 [SMILES: C/C(N)=N\C(=N\S(=O)(=O)c1ccccc1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)((phenylsulfonyl)imino)methyl)carbamimidothioate (11g)

Following general procedure E, starting material (150 mg) gave final compound (87 mg, 49%) as a white solid. LRMS 526.1, HRMS (ESI) m/z: [M+H]+Calcd for (C25 H25 N5 O2 S2 Cl) 526.1138; Found 526.1132.

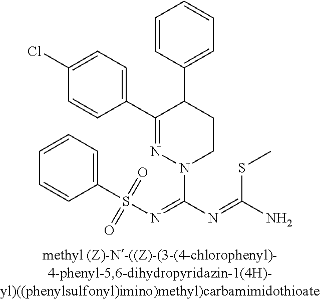 [SMILES: CS/C(N)=N\C(=N\S(=O)(=O)c1ccccc1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)((phenylsulfonyl)imino)methyl)carbamimidoselenoate (12g)

Following general procedure F, starting material (150 mg) gave final compound (89 mg, 47%) as a white solid. LRMS 574.1, HRMS (ESI) m/z: [M+H]+Calcd for (C25 H25 N5 O2 S Cl Se) 574.0583; Found 574.0582.

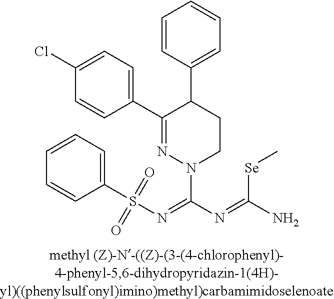 [SMILES: C[Se]/C(N)=N\C(=N\S(=O)(=O)c1ccccc1)N1CCC(c2ccccc2)C(c2ccc(Cl)cc2)=N1]

### Synthesis of (Z)—N—((E)-1-aminoethylidene)-3-(4-chlorophenyl)-N′-(naphthalen-2-ylsulfonyl)-4-phenyl-5,6-dihydropyridazine-1 (4H)-carboximidamide (10f)

Following general procedure D, starting material (300 mg) gave final compound 30 (129 mg, 39.8%) as a white solid. 1H-NMR (800 MHz; CDCl3): δ 8.51 (s, 1H), 7.99 (d, J=8.6 Hz, 1H), 7.93 (d, J=8.0 Hz, 1H), 7.87 (dd, J=21.9, 8.2 Hz, 2H), 7.57-7.50 (m, 4H), 7.26 (t, J=6.8 Hz, 2H), 7.22 (t, J=7.2 Hz, 1H), 7.16 (d, J=8.5 Hz, 2H), 7.04 (d, J=7.3 Hz., 2H), 5.42 (s, 2H), 4.53 (d, J=12.7 Hz, 1H), 4.12 (s, 1H), 3.23 (t, J=13.5 Hz, 1H), 2.15 (dd, J=32.9, 19.8 Hz, 1H), 2.06 (t, J=13.6 Hz, 1H), 1.97 (s, 3H). LRMS 544.2, HRMS (ESI) m/z: [M+H]+Calcd for (C29 H27 N5 O2 S Cl) 544.1574; Found 544.1570.

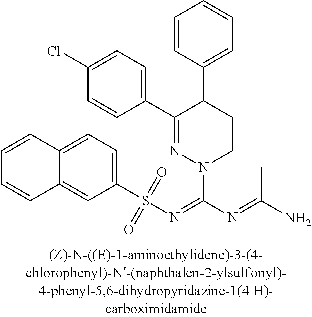 [SMILES: C/C(N)=N\C(=N\S(=O)(=O)c1ccc2ccccc2c1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)((naphthalen-2-ylsulfonyl)imino)methyl)carbamimidothioate (11f)

Following general procedure E, starting material (150 mg) gave final compound 31 (86 mg. 50%) as a white solid. 1H-NMR (800 MHZ; CDCl3: δ 8.52 (s, 1H), 7.99 (d, J=8.5 Hz, 1H), 7.95 (d, J=7.9 Hz, 1H), 7.90 (d, J=8.6 Hz, 1H), 7.87 (d, J=8.1 Hz, 1H), 7.57 (dt, J=21.1, 7.2 Hz, 4H), 7.28 (t, J=7.4 Hz, 2H), 7.23 (t, J=7.2 Hz, 1H), 7.17 (d, J=8.6 Hz, 2H), 7.08 (d, J=7.2 Hz, 2H), 5.30 (s, 1H), 4.53 (d, J=13.2 Hz, 1H), 4.14 (s, 1H), 3.27 (t, J=13.0 Hz, 1H), 2.23 (t, J=13.3 Hz, 1H), 2.11 (s, 3H), 2.07 (d, J=13.4 Hz, 1H). LRMS 576.1, HRMS (ESI) m/z: [M+H]+Calcd for (C29 H27 N5 O2 S2 Cl) 576.1295; Found 576.1296.

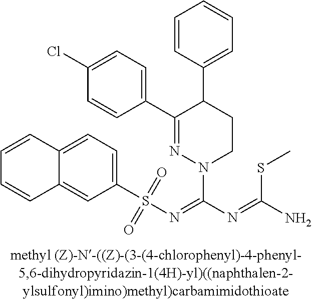 [SMILES: CS/C(N)=N\C(=N\S(=O)(=O)c1ccc2ccccc2c1)N1CCC(c2ccccc2)C(c2ccc(Cl)cc2)=N1]

### Synthesis of methyl (Z)—N′—((Z)-(3-(4-chlorophenyl)-4-phenyl-5,6-dihydropyridazin-1 (4H)-yl)((naphthalen-2-ylsulfonyl)imino)methyl)carbamimidoselenoate (12f)

Following general procedure F, starting material (150 mg) gave final compound 32 (26 mg, 14%) as a white solid. 1H-NMR (800 MHz; CDCl3): δ 8.54 (s, 1H), 8.00 (d, J=8.6 Hz, 1H), 7.96 (d, J=8.1 Hz, 1H), 7.90 (d, J=8.5 Hz, 1H), 7.87 (d, J=7.8 Hz, 1H), 7.57 (dt, J=14.3, 7.0 Hz, 4H), 7.28 (d, J=7.3 Hz, 2H), 7.23 (t, J=7.2 Hz, 1H), 7.17 (d, J=8.6 Hz, 2H), 7.07 (d, J=7.2 Hz, 2H), 5.59 (s, 2H), 4.51 (d, J=13.4 Hz, 1H), 4.14 (s, 1H), 3.26 (t, J=12.7 Hz, 1H), 2.21 (t, J=14.1 Hz, 1H), 2.06 (d, J=13.9 Hz, 1H), 2.03 (s, 3H). LRMS 624.1, HRMS (ESI) m/z: [M+H]+Calcd for (C29 H27 N5 O2 S Cl Se) 624.0739; Found 624.0743.

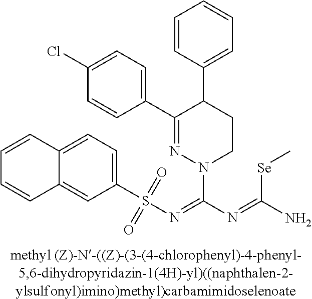 [SMILES: C[Se]/C(N)=N\C(=N\S(=O)(=O)c1ccc2ccccc2c1)N1CCC(c2ccccc2)C(c2ccc(Cl)cc2)=N1]

### Synthesis of (Z)—N—((E)-1-aminoethylidene)-3-(4-chlorophenyl)-N′-((4-methoxyphenyl)sulfonyl)-4-phenyl-5,6-dihydropyridazine-1 (4H)-carboximidamide (10h)

Following general procedure D, starting material (300 mg) gave final compound (75 mg, 24%) as a white solid. LRMS 524.2, HRMS (ESI) m/z: [M+H]+Calcd for (C26 H27 N5 O3 S Cl) 524.1523; Found 524.1515.

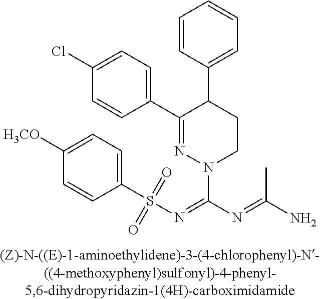 [SMILES: COc1ccc(S(=O)(=O)/N=C(/N=C(\C)N)N2CCC(c3ccccc3)C(c3ccc(Cl)cc3)=N2)cc1]

### Synthesis of methyl (Z)—N′—((Z)-(3-(4-chlorophenyl)-4-phenyl-5,6-dihydropyridazin-1 (4H)-yl)(((4-methoxyphenyl)sulfonyl)imino)methyl)carbamimidothioate (11h)

Following general procedure E, starting material (150 mg) gave final compound (61 mg, 37%) as a white solid. LRMS 556.1, HRMS (EST) m/z: [M+H]+Calcd for (C26 H27 N5 O3 S2 Cl) 556.1244; Found 556.1246.

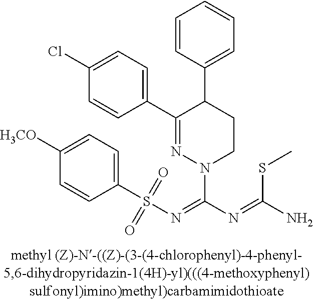 [SMILES: COc1ccc(S(=O)(=O)/N=C(/N=C(/N)SC)N2CCC(c3ccccc3)C(c3ccc(Cl)cc3)=N2)cc1]

### Synthesis of methyl (Z)—N′—((Z)-(3-(4-chlorophenyl)-4-phenyl-5,6-dihydropyridazin-1 (4H)-yl)(((4-methoxyphenyl)sulfonyl)imino)methyl)carbamimidoselenoate (12h)

Following general procedure F, starting material (150 mg) gave final compound (61 mg, 34%) as a white solid. LRMS 604.1, HRMS (ESI) m/z: [M+H]+Calcd for (C26 H27 N5 O3 S Cl Se) 604.0688; Found 604.0696.

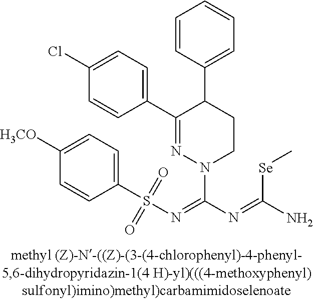 [SMILES: COc1ccc(S(=O)(=O)/N=C(/N=C(/N)[Se]C)N2CCC(c3ccccc3)C(c3ccc(Cl)cc3)=N2)cc1]

### Synthesis of ethyl (Z)—N′—((E)-(3-(4-chlorophenyl)-4-phenyl-5,6-dihydropyridazin-1 (4H)-yl)(((4-(trifluoromethyl)phenyl)sulfonyl)imino)methyl)carbamimidothioate (11i)

Following general procedure E, starting material (300 mg) gave final compound (260 mg, 74.5%) as a white solid. LRMS 608.1, HRMS (ESI) m/z: [M+H]+Calcd for (C27 H26 N5 O2 F3 S2 Cl) 608.1169; Found 608.1162.

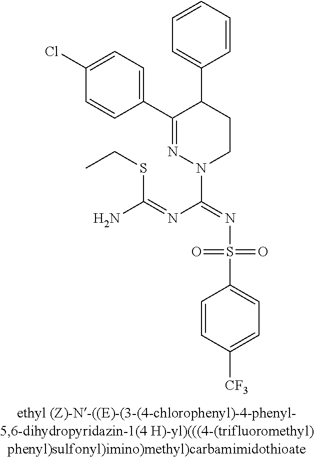 [SMILES: CCS/C(N)=N\C(=N/S(=O)(=O)c1ccc(C(F)(F)F)cc1)N1CCC(c2ccccc2)C(c2ccc(Cl)cc2)=N1]

### Synthesis of methyl (E)-N′—((Z)-(3-(4-fluorophenyl)-4-phenyl-5,6-dihydropyridazin-1 (4H)-yl)(((4-(trifluoromethyl)phenyl)sulfonyl)imino)methyl)carbamimidothioate (11j)

Following general procedure D, starting material (210 mg) gave final compound (101 mg, 42%) as a white solid. LRMS 578.1, HRMS (EST) m/z: [M+H]+Calcd for (C26 H24 N5 O2 F4 S2) 578.1308; Found 578.1311.

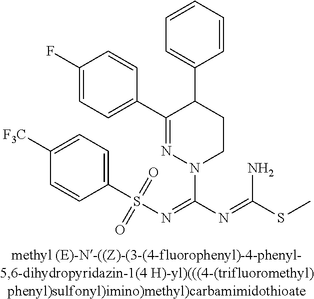 [SMILES: CS/C(N)=N/C(=N/S(=O)(=O)c1ccc(C(F)(F)F)cc1)N1CCC(c2ccccc2)C(c2ccc(F)cc2)=N1]

### Synthesis of methyl (E)-N′—((Z)-(3-(4-fluorophenyl)-4-phenyl-5,6-dihydropyridazin-1 (4H)-yl)(((4-(trifluoromethyl)phenyl)sulfonyl)imino)methyl)carbamimidoselenoate (12i)

Following general procedure D, starting material (210 mg) gave final compound (90 mg, 34.7%) as a white solid. LRMS 626.1, HRMS (ESI) m/z: [M+H]+Calcd for (C26 H24 N5 O2 F4 S Se) 626.0752; Found 626.0750.

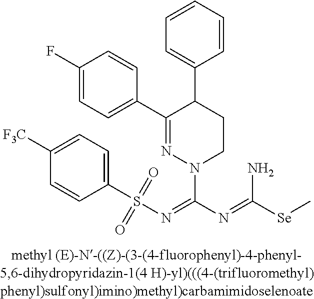 [SMILES: C[Se]/C(N)=N/C(=N/S(=O)(=O)c1ccc(C(F)(F)F)cc1)N1CCC(c2ccccc2)C(c2ccc(F)cc2)=N1]

### Synthesis of (Z)—N—((E)-1-aminoethylidene)-3-(4-chlorophenyl)-N′-((4-iodophenyl)sulfonyl)-4-phenyl-5,6-dihydropyridazine-1 (4H)-carboximidamide (10i)

Following general procedure D, starting material (250 mg) gave final compound (33 mg, 12.4%) as a white solid. LRMS 620.1, HRMS (ESI) m/z: [M+H]+Calcd for (C25 H24 N5 O2 S Cl I) 620.0384; Found 620.0393.

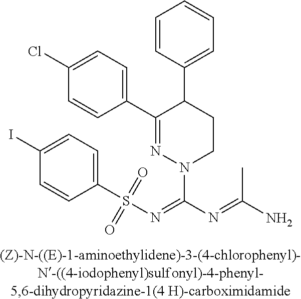 [SMILES: C/C(N)=N\C(=N\S(=O)(=O)c1ccc(I)cc1)N1CCC(c2ccccc2)C(c2ccc(Cl)cc2)=N1]

### Synthesis of (Z)—N—((E)-1-aminoethylidene)-3-(4-chlorophenyl)-N′-((4-fluorophenyl)sulfonyl)-4-phenyl-5,6-dihydropyridazine-1 (4H)-carboximidamide (10j)

Following general procedure D, starting material (300 mg) gave final compound (50 mg, 15.3%) as a white solid. LRMS 512.2, HRMS (ESI) m/z: [M+H]+Calcd for (C25 H24 N5 O2 F S Cl) 512.1323; Found 512.1321.

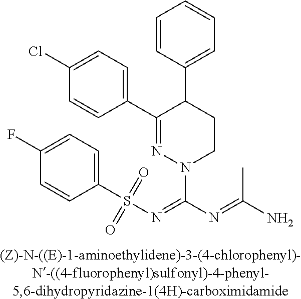 [SMILES: C/C(N)=N\C(=N\S(=O)(=O)c1ccc(F)cc1)N1CCC(c2ccccc2)C(c2ccc(Cl)cc2)=N1]

### Synthesis of (Z)—N—((E)-1-aminoethylidene)-3-(4-chlorophenyl)-4-phenyl-N′-((2-(trifluoromethyl)phenyl)sulfonyl)-5,6-dihydropyridazine-1 (4H)-carboximidamide (10k)

Following general procedure D, starting material (300 mg) gave final compound (84 mg, 26%) as a white solid. LRMS 562.2, HRMS (ESI) m/z: [M+H]+Calcd for (C26 H24 N5 O2 F3 S Cl) 562.1291; Found 562.1295.

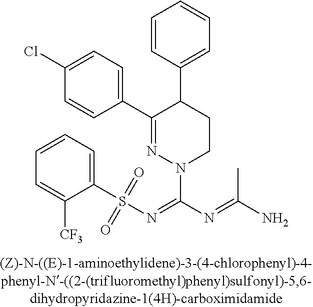 [SMILES: C/C(N)=N\C(=N\S(=O)(=O)c1ccccc1C(F)(F)F)N1CCC(c2ccccc2)C(c2ccc(Cl)cc2)=N1]

### Synthesis of (Z)—N—((E)-1-aminoethylidene)-3-(4-chlorophenyl)-4-phenyl-N′-tosyl-5,6-dihydropyridazine-1 (4H)-carboximidamide (101)

Following general procedure D, starting material (300 mg) gave final compound (26 mg, 8%) as a white solid. LRMS 508.2, HRMS (ESI) m/z: [M+H]+Calcd for (C26 H27 N5 O2 S Cl) 508.1574; Found 508.1572.

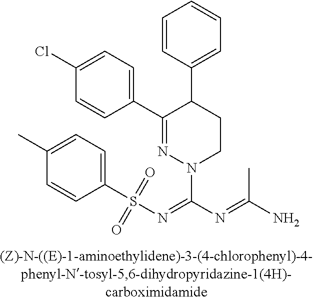 [SMILES: C/C(N)=N\C(=N\S(=O)(=O)c1ccc(C)cc1)N1CCC(c2ccccc2)C(c2ccc(Cl)cc2)=N1]

### Synthesis of (Z)—N—((E)-1-amino-2-methylpropylidene)-3-(4-chlorophenyl)-4-phenyl-N′-((4-(trifluoromethyl)phenyl)sulfonyl)-5,6-dihydropyridazine-1 (4H)-carboximidamide (10m)

Following general procedure D, starting material (275 mg) gave final compound (136 mg, 43.7%) as a white solid. LRMS 590.2, HRMS (ESI) m/z: [M+H]+Calcd for (C28 H28 N5 O2 F3 S Cl) 590.1604; Found 590.1605.

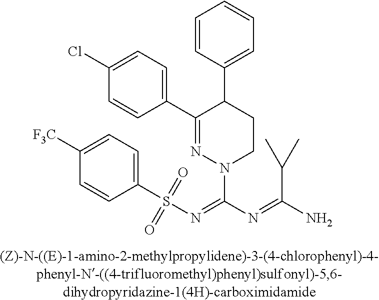 [SMILES: CC(C)/C(N)=N\C(=N\S(=O)(=O)c1ccc(C(F)(F)F)cc1)N1CCC(c2ccccc2)C(c2ccc(Cl)cc2)=N1]

### Synthesis of (Z)—N—((Z)-amino (cyclopropyl)methylene)-3-(4-chlorophenyl)-4-phenyl-N′-((4-(trifluoromethyl)phenyl)sulfonyl)-5,6-dihydropyridazine-1 (4H)-carboximidamide (10n)

Following general procedure D, starting material (275 mg) gave final compound (95 mg, 30.6%) as a white solid. LRMS 588.1, HRMS (ESI) m/z: [M+H]+Calcd for (C28 H26 N5 O2 F3 S Cl) 588.1448; Found 588.1440.

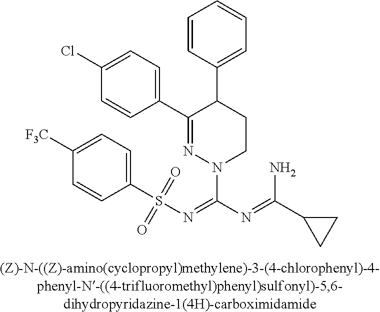 [SMILES: N/C(=N\C(=N\S(=O)(=O)c1ccc(C(F)(F)F)cc1)N1CCC(c2ccccc2)C(c2ccc(Cl)cc2)=N1)C1CC1]

### Synthesis of (Z)—N—((Z)-1-amino-2,2-dimethylpropylidene)-3-(4-chlorophenyl)-4-phenyl-N′-((4-(trifluoromethyl)phenyl)sulfonyl)-5,6-dihydropyridazine-1 (4H)-carboximidamide (10o)

Following general procedure D, starting material (320 mg) gave final compound (170 mg, 44%) as a white solid. 1H NMR (800 MHZ, CDCl₃) δ 8.09 (d, J=8.2 Hz, 2H), 7.71 (d, J=8.4 Hz, 2H), 7.56 (s, 1H), 7.33-7.29 (m, 2H), 7.26 (s, 3H), 7.19 (d, J=9.4 Hz, 2H), 7.09 (d, J=7.6 Hz, 2H), 4.96 (s, 1H), 4.50 (d, J=13.5 Hz, 1H), 4.18 (s, 1H), 3.24 (s, 1H), 2.33-2.03 (m, 2H), 1.29 (s, 9H). LRMS 604.2, HRMS (ESI) m/z: [M+H]+Calcd for (C29 H30 N5 O2 F3 S Cl) 604.1761; Found 604.1766.

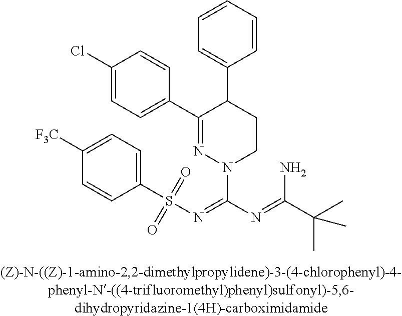 [SMILES: CC(C)(C)/C(N)=N/C(=N/S(=O)(=O)c1ccc(C(F)(F)F)cc1)N1CCC(c2ccccc2)C(c2ccc(Cl)cc2)=N1]

(Z)—N—((Z)-1-amino-2,2-dimethylpropylidene)-3-(4-chlorophenyl)-4-phenyl-N′-((4-(trifluoromethyl)phenyl)sulfonyl)-5,6-dihydropyridazine-1 (4H)-carboximidamide (10o) was chirally separated (Analytical Chiral HPLC (R,R)-Whelk-01 chiral column (250 mm×4.6 mm/5 μm): 80% hexanes: 20% Ethanol (Isocratic, 254 nm), Flow rate: 1 mL/min Peak 1 (10oE1)=12.7 min. Peak 2 (10oE2)=15.1 min.) and first peak 10oE1 eluted was checked for NMR. 1H NMR (800 MHZ, CDCl₃) δ 8.09 (d, J=8.2 Hz, 2H), 7.71 (d, J=8.4 Hz, 2H), 7.56 (s, 1H), 7.33-7.29 (m, 2H), 7.26 (s, 3H), 7.19 (d, J=9.4 Hz, 2H), 7.09 (d, J=7.6 Hz, 2H), 4.96 (s, 1H), 4.50 (d, J=13.5 Hz, 1H), 4.18 (s, 1H), 3.24 (s, 1H), 2.33-2.03 (m, 2H), 1.29 (s, 9H).

And the second peak 10oE2 was checked for NMR, ¹H NMR (800 MHZ, CDCl₃) δ 8.09 (d, J=8.2 Hz, 2H), 7.71 (d, J=8.5 Hz, 2H), 7.55 (s, 2H), 7.33-7.28 (m, 2H), 7.25 (d, J=4.1 Hz, 1H), 7.22-7.16 (m, 2H), 7.09 (d, J=7.2 Hz, 2H), 4.98 (s, 2H), 4.50 (d, J=13.5 Hz, 1H), 4.18 (s, 1H), 3.23 (t, J=11.8 Hz, 1H), 2.29-2.04 (m, 2H), 1.28 (s, 9H).

### Synthesis of (Z)—N—((Z)-amino (2,4-difluorophenyl)methylene)-3-(4-chlorophenyl)-4-phenyl-N′-((4-(trifluoromethyl)phenyl)sulfonyl)-5,6-dihydropyridazine-1 (4H)-carboximidamide (10p)

Following general procedure D, starting material (260 mg) gave final compound (85 mg, 26%) as a white solid. 1H NMR (800 MHZ, CDCl3) δ 8.06 (d, J=8.4 Hz, 2H), 7.87 (s, 1H), 7.68 (d, J=8.2 Hz, 2H), 7.44 (s, 2H), 7.31-7.22 (m, 6H), 7.12 (d, J=8.7 Hz, 2H), 7.02-6.83 (m, 4H), 5.87 (s, 2H), 4.52 (d, J=13.5 Hz, 1H), 4.13 (s, 1H), 3.20 (s, 1H), 2.21-1.97 (m, 2H). LRMS 660.1, HRMS (ESI) m/z: [M+H]+Calcd for (C₃₁ H₂₄ N₅ O₂ F₅ S Cl) 660.1259; Found 660.1259.

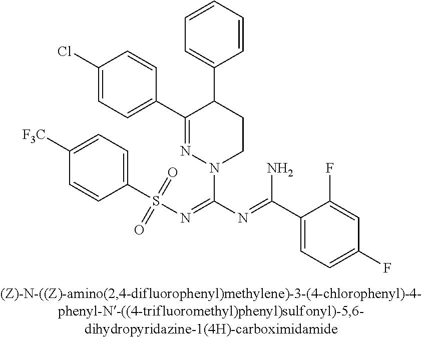 [SMILES: N/C(=N\C(=N\S(=O)(=O)c1ccc(C(F)(F)F)cc1)N1CCC(c2ccccc2)C(c2ccc(Cl)cc2)=N1)c1ccc(F)cc1F]

### Synthesis of (Z)—N—((Z)-((1S,3s)-adamantan-1-yl) (amino)methylene)-3-(4-chlorophenyl)-4-phenyl-N′-((4-(trifluoromethyl)phenyl)sulfonyl)-5,6-dihydropyridazine-1 (4H)-carboximidamide (10q)

Following general procedure D, starting material (260 mg) gave final compound (103 mg, 30.3%) as a white solid. LRMS 682.2, HRMS (ESI) m/z: [M+H]+Calcd for (C35 H36 N5 O2 F3 S Cl) 682.2230; Found 682.2240.

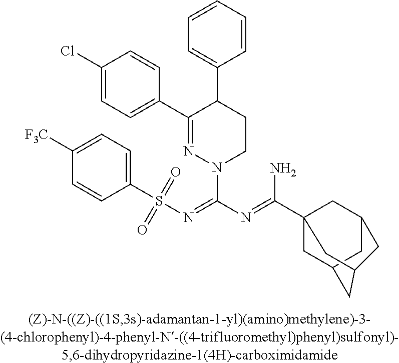 [SMILES: N/C(=N\C(=N\S(=O)(=O)c1ccc(C(F)(F)F)cc1)N1CCC(c2ccccc2)C(c2ccc(Cl)cc2)=N1)C12CC3CC(CC(C3)C1)C2]

### Synthesis of (Z)—N—((Z)-amino (phenyl)methylene)-3-(4-chlorophenyl)-4-phenyl-N′-((4-(trifluoromethyl)phenyl)sulfonyl)-5,6-dihydropyridazine-1 (4H)-carboximidamide (10r)

Following general procedure D, starting material (275 mg) gave final compound (160 mg, 48.6%) as a white solid. LRMS 624.1, HRMS (ESI) m/z: [M+H]+Calcd for (C31 H26 N5 O2 F3 S Cl) 624.1448; Found 624.1447.

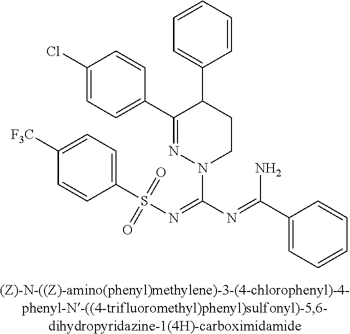 [SMILES: N/C(=N\C(=N\S(=O)(=O)c1ccc(C(F)(F)F)cc1)N1CCC(c2ccccc2)C(c2ccc(Cl)cc2)=N1)c1ccccc1]

### Synthesis of (Z)—N—((E)-amino (2-chlorophenyl)methylene)-3-(4-chlorophenyl)-4-phenyl-N′-((4-(trifluoromethyl)phenyl)sulfonyl)-5,6-dihydropyridazine-1 (4H)-carboximidamide (10s)

Following general procedure D, starting material (300 mg) gave final compound (115.7 mg, 30.5%) as a white solid. LRMS 658.1, HRMS (ESI) m/z: [M+H]+Calcd for (C31 H25 N5 O2 F3 S Cl2) 658.1058; Found 658.1047.

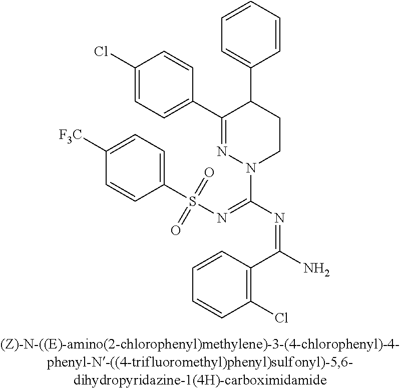 [SMILES: N/C(=N/C(=N/S(=O)(=O)c1ccc(C(F)(F)F)cc1)N1CCC(c2ccccc2)C(c2ccc(Cl)cc2)=N1)c1ccccc1Cl]

### Synthesis of (Z)—N—((Z)-amino (2-fluorophenyl)methylene)-3-(4-chlorophenyl)-4-phenyl-N′-((4-(trifluoromethyl)phenyl)sulfonyl)-5,6-dihydropyridazine-1 (4H)-carboximidamide (10t)

Following general procedure D, starting material (320 mg) gave final compound (120 mg, 30.5%) as a white solid. 1H NMR (800 MHZ, CDCl3) δ 8.07 (d, J=8.2 Hz, 2H), 7.79 (s, 1H), 7.66 (d, J=8.4 Hz, 2H), 7.57-7.39 (m, 3H), 7.26-7.17 (m, 4H), 7.18-7.06 (m, 3H), 6.93 (s, 2H), 5.93 (s, 2H), 4.54 (s, 1H), 4.11 (s, 1H), 3.21 (s, 1H), 2.22-1.98 (m, 2H). LRMS 642.1, HRMS (ESI) m/z: [M+H]+Calcd for (C₃₁ H₂₅ N₅ O₂ F₄ S Cl) 642.1354; Found 642.1353.

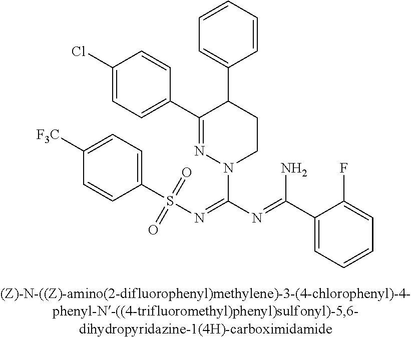 [SMILES: N/C(=N\C(=N\S(=O)(=O)c1ccc(C(F)(F)F)cc1)N1CCC(c2ccccc2)C(c2ccc(Cl)cc2)=N1)c1ccccc1F]

The compound (Z)—N—((Z)-amino (2-fluorophenyl)methylene)-3-(4-chlorophenyl)-4-phenyl-N′-((4-(trifluoromethyl)phenyl)sulfonyl)-5,6-dihydropyridazine-1 (4H)-carboximidamide (10t) was further separated through chiral HPLC to afford two enantiomers 10tE1 and 10tE2.

### Synthesis of (Z)—N—((Z)-amino (4-fluorophenyl)methylene)-3-(4-chlorophenyl)-4-phenyl-N′-((4-(trifluoromethyl)phenyl)sulfonyl)-5,6-dihydropyridazine-1 (4H)-carboximidamide (10u)

Following general procedure D, starting material (320 mg) gave final compound (120 mg, 30.5%) as a white solid. LRMS 642.1, HRMS (ESI) m/z: [M+H]+Calcd for (C31 H25 N5 O2 F4 S Cl) 642.1354; Found 642.1359.

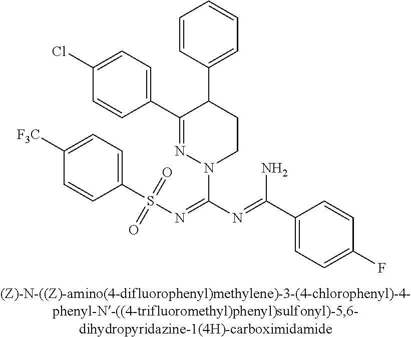 [SMILES: N/C(=N\C(=N\S(=O)(=O)c1ccc(C(F)(F)F)cc1)N1CCC(c2ccccc2)C(c2ccc(Cl)cc2)=N1)c1ccc(F)cc1]

### Synthesis of (Z)—N—((Z)-amino (pyridin-2-yl)methylene)-3-(4-chlorophenyl)-4-phenyl-N′-((4-(trifluoromethyl)phenyl)sulfonyl)-5,6-dihydropyridazine-1 (4H)-carboximidamide (10v)

Following general procedure D, starting material (320 mg) gave final compound (170 mg, 44%) as a white solid. ¹H NMR (800 MHZ, CDCl₃) δ 8.64 (s, 1H), 8.02 (s, 2H), 7.85-7.71 (m, 2H), 7.63 (d, J=7.0 Hz, 2H), 7.50 (s, 1H), 7.39-7.27 (m, 5H), 7.05 (d, J=50.6 Hz, 4H), 4.62 (d, J=12.8 Hz, 1H), 4.18 (s, 1H), 3.32 (s, 1H), 2.34-2.09 (m, 2H). LRMS 625.1, HRMS (ESI) m/z: [M+H]+Calcd for (C₃₀ H₂₅ N₆ O₂ F₃ S Cl) 625.1400; Found 625.1398.

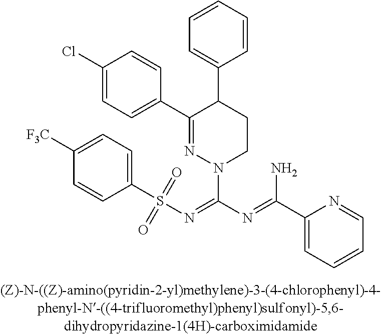 [SMILES: N/C(=N\C(=N\S(=O)(=O)c1ccc(C(F)(F)F)cc1)N1CCC(c2ccccc2)C(c2ccc(Cl)cc2)=N1)c1ccccn1]

The compound (Z)—N—((Z)-amino (pyridin-2-yl)methylene)-3-(4-chlorophenyl)-4-phenyl-N′-((4-(trifluoromethyl)phenyl)sulfonyl)-5,6-dihydropyridazine-1 (4H)-carboximidamide (10v) was further separated through chiral HPLC to afford two enantiomers 10vE1 and 10vE2.

### Synthesis of (Z)—N—((Z)-amino (thiophen-2-yl)methylene)-3-(4-chlorophenyl)-4-phenyl-N′-((4-(trifluoromethyl)phenyl)sulfonyl)-5,6-dihydropyridazine-1 (4H)-carboximidamide (10w)

Following general procedure D, starting material (320 mg) gave final compound (170 mg, 44%) as a pale white solid. LRMS 630.1, HRMS (ESI) m/z: [M+H]+Calcd for (C29 H24 N5 O2 F3 S2 Cl) 630.1012; Found 630.1015.

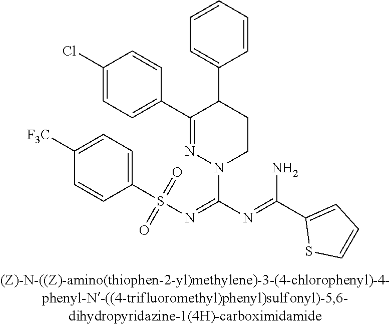 [SMILES: N/C(=N\C(=N\S(=O)(=O)c1ccc(C(F)(F)F)cc1)N1CCC(c2ccccc2)C(c2ccc(Cl)cc2)=N1)c1cccs1]

### Synthesis of (Z)—N—((Z)-1-amino-2,2-dimethylpropylidene)-3-(4-bromophenyl)-4-phenyl-N′-((4-(trifluoromethyl)phenyl)sulfonyl)-5,6-dihydropyridazine-1 (4H)-carboximidamide (10x)

Following general procedure D, starting material (265 mg) gave final compound (110 mg, 36.3%) as a white solid. LRMS 650.1, HRMS (ESI) m/z: [M+H]+Calcd for (C29 H30 N5 O2 F3 S Br) 648.1256; Found 648.1263.

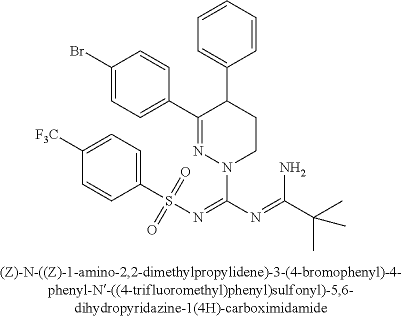 [SMILES: CC(C)(C)/C(N)=N/C(=N/S(=O)(=O)c1ccc(C(F)(F)F)cc1)N1CCC(c2ccccc2)C(c2ccc(Br)cc2)=N1]

### Synthesis of (Z)—N—((Z)-1-aminoethylidene)-3-(4-bromophenyl)-4-phenyl-N′-((4-(trifluoromethyl)phenyl)sulfonyl)-5,6-dihydropyridazine-1 (4H)-carboximidamide (10y)

Following general procedure D, starting material (265 mg) gave final compound (101 mg, 35.6%) as a white solid. LRMS 608.1, HRMS (ESI) m/z: [M+H]+Calcd for (C26 H24 N5 O2 F3 S Br) 606.0786; Found 606.0789.

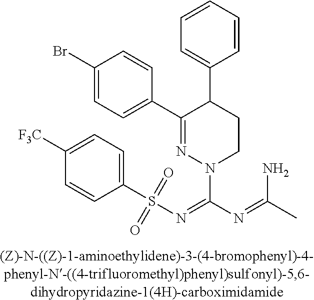 [SMILES: C/C(N)=N/C(=N/S(=O)(=O)c1ccc(C(F)(F)F)cc1)N1CCC(c2ccccc2)C(c2ccc(Br)cc2)=N1]

### Synthesis of (Z)—N—((Z)-amino (2-fluorophenyl)methylene)-3-(4-bromophenyl)-4-phenyl-N′-((4-(trifluoromethyl)phenyl)sulfonyl)-5,6-dihydropyridazine-1 (4H)-carboximidamide (10z)

Following general procedure D, starting material (265 mg) gave final compound (99 mg, 30.8%) as a white solid. LRMS 688.1, HRMS (ESI) m/z: [M+H]+Calcd for (C31 H25 N5 O2 F4 S Br) 686.0848; Found 686.0853.

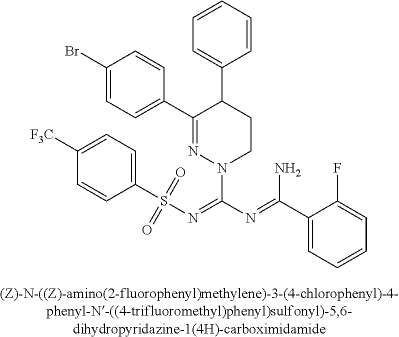 [SMILES: N/C(=N\C(=N\S(=O)(=O)c1ccc(C(F)(F)F)cc1)N1CCC(c2ccccc2)C(c2ccc(Br)cc2)=N1)c1ccccc1F]

### Synthesis of (Z)—N—((Z)-amino (2,4-difluorophenyl)methylene)-3-(4-bromophenyl)-4-phenyl-N′-((4-(trifluoromethyl)phenyl)sulfonyl)-5,6-dihydropyridazine-1 (4H)-carboximidamide (10za)

Following general procedure D, starting material (265 mg) gave final compound (130 mg, 39.5%) as a white solid. LRMS 706.1, HRMS (ESI) m/z: [M+H]+Calcd for (C31 H24 N5 O2 F5 S Br) 704.0754; Found 704.0748.

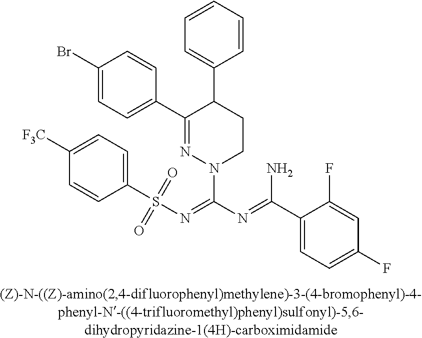 [SMILES: N/C(=N\C(=N\S(=O)(=O)c1ccc(C(F)(F)F)cc1)N1CCC(c2ccccc2)C(c2ccc(Br)cc2)=N1)c1ccc(F)cc1F]

### Synthesis of (Z)—N—((Z)-1-amino-2,2-dimethylpropylidene)-3-(4-fluorophenyl)-4-phenyl-N′-((4-(trifluoromethyl)phenyl)sulfonyl)-5,6-dihydropyridazine-1 (4H)-carboximidamide (10zb)

Following general procedure D, starting material (175 mg) gave final compound (80 mg, 39.3%) as a white solid. LRMS 588.2, HRMS (ESI) m/z: [M+H]+Calcd for (C29 H30 N5 O2 F4 S) 588.2056; Found 588.2057.

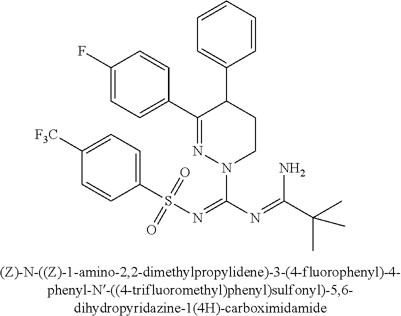 [SMILES: CC(C)(C)/C(N)=N/C(=N/S(=O)(=O)c1ccc(C(F)(F)F)cc1)N1CCC(c2ccccc2)C(c2ccc(F)cc2)=N1]

### Synthesis of (Z)—N—((Z)-1-aminoethylidene)-3-(4-fluorophenyl)-4-phenyl-N′-((4-(trifluoromethyl)phenyl)sulfonyl)-5,6-dihydropyridazine-1 (4H)-carboximidamide (10zc)

Following general procedure D, starting material (175 mg) gave final compound (35 mg, 18.5%) as a white solid. LRMS 546.2, HRMS (ESI) m/z: [M+H]+Calcd for (C26 H24 N5 O2 F4 S) 546.1587; Found 546.1592.

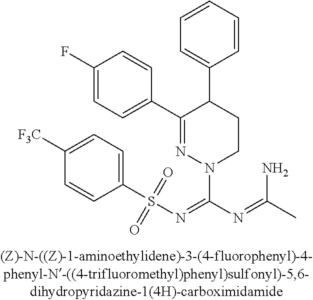 [SMILES: C/C(N)=N/C(=N/S(=O)(=O)c1ccc(C(F)(F)F)cc1)N1CCC(c2ccccc2)C(c2ccc(F)cc2)=N1]

### Synthesis of (Z)—N—((Z)-amino (2-fluorophenyl)methylene)-3-(4-fluorophenyl)-4-phenyl-N′-((4-(trifluoromethyl)phenyl)sulfonyl)-5,6-dihydropyridazine-1 (4H)-carboximidamide (10zd)

Following general procedure D, starting material (200 mg) gave final compound (82 mg, 33.2%) as a white solid. LRMS 626.2, HRMS (ESI) m/z: [M+H]+Calcd for (C31 H25 N5 O2 F5 S) 626.1649; Found 626.1647.

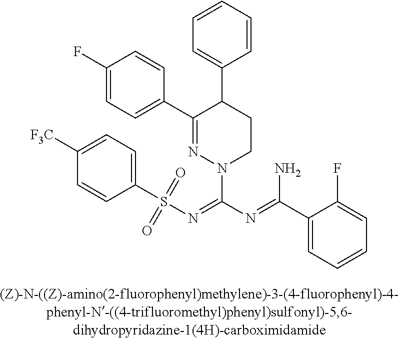 [SMILES: N/C(=N\C(=N\S(=O)(=O)c1ccc(C(F)(F)F)cc1)N1CCC(c2ccccc2)C(c2ccc(F)cc2)=N1)c1ccccc1F]

### Synthesis of (Z)—N—((Z)-amino (2,4-difluorophenyl)methylene)-3-(4-fluorophenyl)-4-phenyl-N′-((4-(trifluoromethyl)phenyl)sulfonyl)-5,6-dihydropyridazine-1 (4H)-carboximidamide (10zc)

Following general procedure D, starting material (210 mg) gave final compound (55 mg, 20.5%) as a white solid. LRMS 644.2, HRMS (ESI) m/z: [M+H]+Calcd for (C31 H24 N5 O2 F6 S) 644.1555; Found 644.1549.

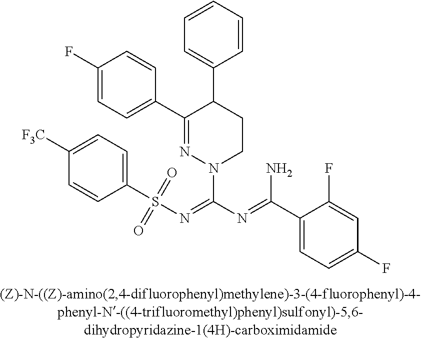 [SMILES: N/C(=N\C(=N\S(=O)(=O)c1ccc(C(F)(F)F)cc1)N1CCC(c2ccccc2)C(c2ccc(F)cc2)=N1)c1ccc(F)cc1F]

### Synthesis of (Z)—N—((Z)-amino (pyridin-2-yl)methylene)-3-(4-fluorophenyl)-4-phenyl-N′-((4-(trifluoromethyl)phenyl)sulfonyl)-5,6-dihydropyridazine-1 (4H)-carboximidamide (10zf)

Following general procedure D, starting material (210 mg) gave final compound (79 mg, 31.2%) as a white solid. LRMS 609.2, HRMS (ESI) m/z: [M+H]+Calcd for (C30 H25 N6 O2 F4 S) 609.1696; Found 609.1703.

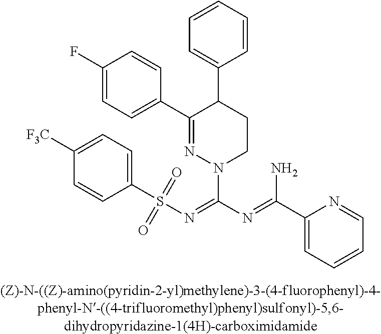 [SMILES: N/C(=N\C(=N\S(=O)(=O)c1ccc(C(F)(F)F)cc1)N1CCC(c2ccccc2)C(c2ccc(F)cc2)=N1)c1ccccn1]

### Synthesis of N—((Z)—N′—((Z)-(3-(4-chlorophenyl)-4-phenyl-5,6-dihydropyridazin-1 (4H)-yl)(((4-cyanophenyl)sulfonyl)imino)methyl) carbamimidoyl) acetamide (13a)

Following general procedure D, starting material (150 mg) gave final compound (45 mg, 25.5%) as a white solid. LRMS 562.1, HRMS (ESI) m/z: [M+H]+Calcd for (C27 H25 N7 O3 S Cl) 562.1428; Found 562.1433.

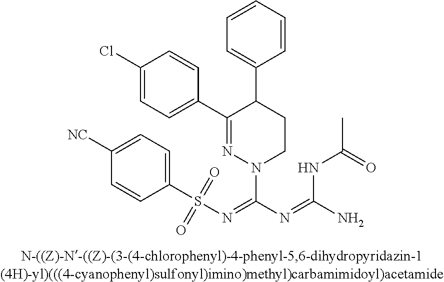 [SMILES: CC(=O)N/C(N)=N\C(=N\S(=O)(=O)c1ccc(C#N)cc1)N1CCC(c2ccccc2)C(c2ccc(Cl)cc2)=N1]

### Synthesis of N—((Z)—N′—((Z)-(3-(4-chlorophenyl)-4-phenyl-5,6-dihydropyridazin-1 (4H)-yl)(((4-(trifluoromethyl)phenyl)sulfonyl)imino)methyl) carbamimidoyl) acetamide (14)

Following general procedure D, starting material (300 mg) gave final compound (85 mg, 26.5%) as a white solid. LRMS 605.1, HRMS (ESI) m/z: [M+H]+Calcd for (C27 H25 N6 O3 F3 S Cl) 605.1349; Found 605.1352.

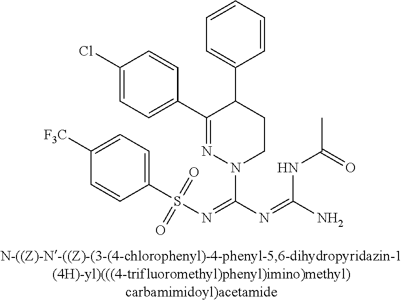 [SMILES: CC(=O)N/C(N)=N\C(=N\S(=O)(=O)c1ccc(C(F)(F)F)cc1)N1CCC(c2ccccc2)C(c2ccc(Cl)cc2)=N1]

### Synthesis of (Z)—N—((E)-amino (diethylamino)methylene)-3-(4-chlorophenyl)-4-phenyl-N′-((4-(trifluoromethyl)phenyl)sulfonyl)-5,6-dihydropyridazine-1 (4H)-carboximidamide (18)

70 mg of Methyl (Z)—N′—((Z)-(3-(4-chlorophenyl)-4-phenyl-5,6-dihydropyridazin-1 (4H)-yl)(((4-(trifluoromethyl)phenyl)sulfonyl)imino)methyl)carbamimidothioate (11b) dissolved in Pyridine/diisopropylamine in a 2-neck reaction flask fitted with a reflux condenser and continued for 20 hours. The reaction mixture was extracted into dichloromethane, washed with water, and purified by flash chromatography using hexanes/EtOAC (1:1) followed by trituration with IPA/hexane to afford compound 18 (40 mg, 54%) as white solid. LRMS 619.2, HRMS (ESI) m/z: [M+H]+Calcd for (C29 H31 N6 O2 F3 S Cl) 619.1870; Found 619.1874.

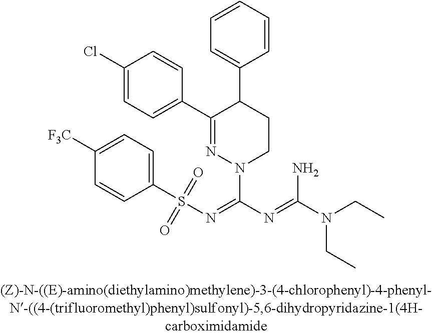 [SMILES: CCN(CC)/C(N)=N/C(=N/S(=O)(=O)c1ccc(C(F)(F)F)cc1)N1CCC(c2ccccc2)C(c2ccc(Cl)cc2)=N1]

### Synthesis of tert-butyl 2-((Z)-2-((Z)-(3-(4-chlorophenyl)-4-phenyl-5,6-dihydropyridazin-1 (4H)-yl)(((4-(trifluoromethyl)phenyl)sulfonyl)imino)methyl) guanidino)-2-oxoacetate (19)

Following general procedure D, starting material (350 mg) gave final compound (198 mg, 44.5%) as a white solid. LRMS 663.2, HRMS (ESI) m/z: [M+H]+Calcd for (C30 H31 N6 O4 F3 S Cl) 663.1768; Found 663.1765.

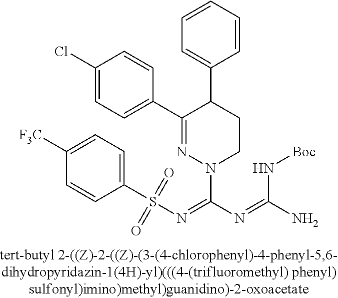 [SMILES: CC(C)(C)OC(=O)N/C(N)=N\C(=N\S(=O)(=O)c1ccc(C(F)(F)F)cc1)N1CCC(c2ccccc2)C(c2ccc(Cl)cc2)=N1]

### Synthesis of (Z)-3-(4-chlorophenyl)-N′-((4-cyanophenyl)sulfonyl)-4-phenyl-N—((E)-piperidin-2-ylidene)-5,6-dihydropyridazine-1 (4H)-carboximidamide (13b)

To a mixture of compound 8e (300 mg, 0.62 mmol) in toluene (2 mL), POCl₃ (175 μL, 1.88 mmol), was added, followed by the addition of DIPEA (436 μL, 2.5 mmol) and the mixture was refluxed for 5 h. The reaction mixture was then cooled, and toluene was evaporated in vacuo. The crude imidoyl chloride compound was dissolved in dichloromethane (1 mL), and to it was added a premixed mixture of 2-iminopiperidine hydrochloride (380 mg, 2.82 mmol) in methanol/dichloromethane (1:4, 2 mL) and Et₃N (524 μL, 3.76 mmol) at room temperature dropwise. The mixture was allowed to warm up to room temperature and stirred for 18 hrs. The reaction mixture was extracted into dichloromethane, washed with water, and purified by flash chromatography using hexanes/EtOAC (4:6) followed by trituration with IPA/hexane to afford compound 13b (76 mg, 21.7%) as yellowish white solid. 1H-NMR (800 MHZ; CDCl3): δ 8.06 (d, J=8.1 Hz, 2H), 7.72 (d, J=8.2 Hz, 2H), 7.54 (d, J=8.1 Hz, 2H), 7.30 (1, J=7.4 Hz, 2H), 7.25 (d, J=7.5 Hz, 1H), 7.20 (d, J=8.6 Hz, 2H), 7.06 (d, J=7.4 Hz, 2H), 4.50 (d, J=13.8 Hz, 1H), 4.16 (s, 1H), 3.42 (s, 2H), 3.22 (t, J=12.1 Hz, 1H), 2.39 (d, J=10.6 Hz, 2H), 2.17 (d, J=13.3 Hz, 1H), 2.08 (d, J=13.7 Hz, 1H), 1.79-1.72 (m, 4H). LRMS 559.1, HRMS (ESI) m/z: [M+H]+Calcd for (C29 H28 N6 O2 S Cl) 559.1683; Found 559.1688.

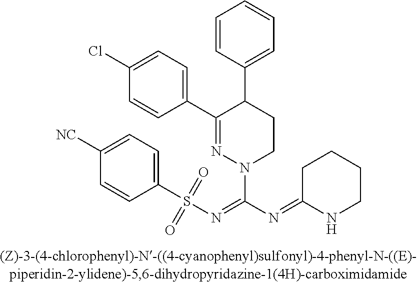 [SMILES: N#Cc1ccc(S(=O)(=O)/N=C(/N=C2\CCCCN2)N2CCC(c3ccccc3)C(c3ccc(Cl)cc3)=N2)cc1]

### Synthesis of (Z)-3-(4-chlorophenyl)-N′-(naphthalen-2-ylsulfonyl)-4-phenyl-N—((E)-piperidin-2-ylidene)-5,6-dihydropyridazine-1 (4H)-carboximidamide (15)

Following the above general procedure, starting material (300 mg) gave final compound (99.5 mg, 28.6%) as a light brown solid. 1H-NMR (800 MHz; CDCl3): δ 8.52 (s, 1H), 8.00 (d, J=8.4 Hz, 1H), 7.95 (d, J=7.7 Hz, 1H), 7.89 (d, J=8.5 Hz, 1H), 7.86 (d, J=7.8 Hz, 1H), 7.57-7.50 (m, 4H), 7.28 (t, J=7.3 Hz, 2H), 7.23 (t, J=7.3 Hz, 1H), 7.14 (d, J=8.5 Hz, 2H), 7.05 (d, J=7.4 Hz, 2H), 4.56 (d, J=13.7 Hz, 1H), 4.12 (s, 1H), 3.26-3.22 (m, 3H), 2.20-2.12 (m, 3H), 2.06-2.04 (m, 1H), 1.59 (t, J=5.2 Hz, 4H). LRMS 584.2, HRMS (ESI) m/z: [M+H]+Calcd for (C32 H31 N5 O2 S Cl) 584.1887; Found 584.1893.

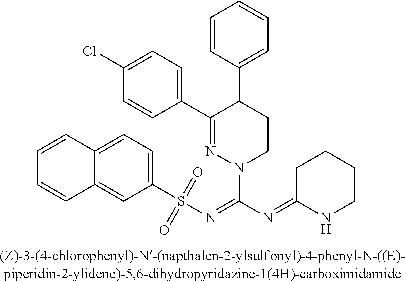 [SMILES: O=S(=O)(/N=C(/N=C1\CCCCN1)N1CCC(c2ccccc2)C(c2ccc(Cl)cc2)=N1)c1ccc2ccccc2c1]

### Synthesis of (Z)-3-(4-chlorophenyl)-4-phenyl-N—((E)-piperidin-2-ylidene)-N′-((4-(trifluoromethyl)phenyl)sulfonyl)-5,6-dihydropyridazine-1 (4H)-carboximidamide (16)

Following the above general procedure, starting material (300 mg) gave final compound (92 mg, 26.6%) as a white solid. LRMS 602.2, HRMS (ESI) m/z: [M+H]+Calcd for (C29 H28 N5 O2 F3 S Cl) 602.1604; Found 602.1605.

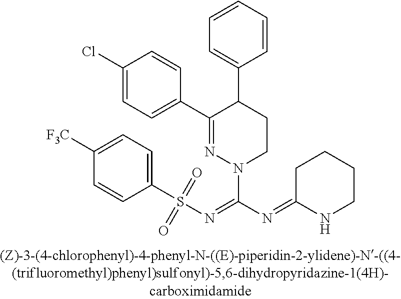 [SMILES: O=S(=O)(/N=C(/N=C1\CCCCN1)N1CCC(c2ccccc2)C(c2ccc(Cl)cc2)=N1)c1ccc(C(F)(F)F)cc1]

### Synthesis of (E)-3-(4-chlorophenyl)-4-phenyl-N—((E)-pyrrolidin-2-ylidene)-N′-((4-(trifluoromethyl)phenyl)sulfonyl)-5,6-dihydropyridazine-1 (4H)-carboximidamide (17)

Following the above general procedure, starting material (300 mg) gave final compound (10 mg, 3%) as a white solid. LRMS 588.1, HRMS (ESI) m/z: [M+H]+Calcd for (C28 H26 N5 O2 F3 S Cl) 588.1448; Found 588.1445.

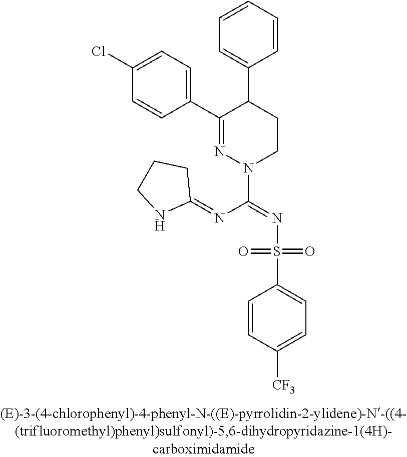 [SMILES: O=S(=O)(/N=C(\N=C1/CCCN1)N1CCC(c2ccccc2)C(c2ccc(Cl)cc2)=N1)c1ccc(C(F)(F)F)cc1]

### Synthesis of 3-(4-Chlorophenyl)-N-((4-cyanophenyl)sulfonyl)-4-phenyl-5,6-dihydropyridazine-1 (4H)-carbothioamide (9c)

Carboxamide Compound 8e (1 equiv.) was dissolved in dry toluene (5 mL). To the solution was added N, N-diisopropylethylamine (2 equiv.) under nitrogen atmosphere. POCl3 (1.5 equiv.) was added to the reaction mixture under ice cold condition, and it was refluxed for 3 hours. Completion of the reaction was confirmed by thin layer chromatography. Toluene was evaporated and intermediate was taken to the next step as is. A solution of sodium thiosulfate (2 equiv.) dissolved in a 4 mL mixture of dioxane and water (8:1) was added dropwise to the reaction mixture. The reaction mixture was heated at 90° C. for 2 hours. The solvent was removed under vacuum, extracted with DCM, and washed with brine solution. The residue was purified by silica gel column chromatography to provide compounds as white solid. Following general procedure, starting urea compound (100 mg, 0.21 mmol) was converted in a two-step, one-pot protocol to title compound thiourea 9c (49 mg, 48% yield) as a pale yellow solid. 1H-NMR (800 MHz; CDCl3): δ 10.43 (s, 1H), 8.28 (d, J=8.3 Hz, 2H), 7.85 (d, J=8.5 Hz, 2H), 7.60 (d, J=8.5 Hz, 2H), 7.33 (t, J=7.8 Hz, 4H), 7.28 (d, J=7.4 Hz, 1H), 7.07 (d, J=7.2 Hz, 2H), 4.92 (d, J=12.1 Hz, 1H), 4.27 (s, 1H), 3.20 (td, J=13.5, 3.4 Hz, 1H), 2.21-2.14 (m, 2H). 13-C NMR (201 MHz; CDCl3): δ 173.5, 150.0, 142.8, 139.5, 137.0, 133.6, 132.5, 130.2, 129.6, 129.3, 128.9, 127.9, 117.4, 39.2, 38.4, 26.3. LRMS 495.0, HRMS (C24 H19 CI N4 O2 S2) [M+H]+found m/z 495.0714, and calcd 495.0716.

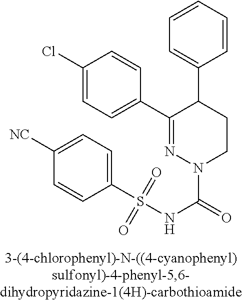 [SMILES: N#Cc1ccc(S(=O)(=O)NC(=O)N2CCC(c3ccccc3)C(c3ccc(Cl)cc3)=N2)cc1]

### Synthesis of 3-(4-chlorophenyl)-N-((4-chlorophenyl)sulfonyl)-4-phenyl-5,6-dihydropyridazine-1 (4H)-carbothioamide (9a)

Following above procedure, starting urea compound (50 mg, 0.05 mmol) was converted in a two-step, one-pot protocol to title compound thiourea 9a (19 mg, 38% yield) as a pale solid. 1H-NMR (800 MHz; CDCl3): § 10.39 (s, 1H), 8.11 (d, J=8.8 Hz, 2H), 7.59 (d, J=8.7 Hz, 2H), 7.52 (d, J=8.8 Hz, 2H), 7.31 (t, J=16.0 Hz, 5H), 7.06 (d, J=7.7 Hz, 2H), 4.94 (d, J=10.7 Hz, 1H), 4.25 (s, 1H), 3.20 (td, J=13.5, 3.9 Hz, 1H), 2.18-2.13 (m, 2H). 13-C NMR (201 MHz; CDCl3): δ 173.6, 149.7, 139.76, 139.61, 136.8, 133.7, 133.1, 131.5, 130.5, 129.52, 129.36, 126.6, 39.1, 38.4, 26.3. LRMS 504.1, HRMS (C23 H20 C12 N3 O2 S2) [M+H]+found m/z 504.0368, and calcd 504.0374.

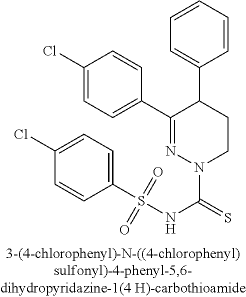 [SMILES: O=S(=O)(NC(=S)N1CCC(c2ccccc2)C(c2ccc(Cl)cc2)=N1)c1ccc(Cl)cc1]

### Synthesis of 3-(4-chlorophenyl)-N-((4-cyanophenyl)sulfonyl)-4-phenyl-5,6-dihydropyridazine-1 (4H)-carbothioamide (9b)

Following the above procedure, starting urea compound (100 mg, 0.19 mmol) was converted in a two-step, one-pot protocol to title compound thiourea 9b (44.5 mg, 43% yield) as a pale solid. 1H-NMR (800 MHZ; CDCl3): δ 10.44 (s, 1H), 8.43-8.41 (m, 2H), 7.90 (d, J=7.7 Hz, 1H), 7.72 (t, J=7.9 Hz, 1H), 7.62 (d, J=8.7 Hz, 2H), 7.35-7.33 (m, 4H), 7.29 (d, J=7.4 Hz, 1H), 7.08 (d, J=7.4 Hz, 2H), 4.95 (d, J=13.8 Hz, 1H), 4.28 (d, J=4.3 Hz, 1H), 3.21 (td, J=13.6, 4.0 Hz, 1H), 2.24-2.14 (m, 2H). 13C-NMR (201 MHz; CDCl3): δ 173.8, 149.4, 140.6, 139.6, 137.0, 136.8, 133.7, 131.1, 129.5, 129.3, 129.0, 128.7, 127.9, 127.4, 39.1, 38.4, 26.4. LRMS 538.1, HRMS (C24 H20 Cl F3 N3 O2 S2) [M+H]+found m/z 538.0643, and calcd 538.0638.

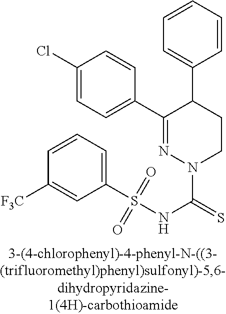 [SMILES: O=S(=O)(NC(=S)N1CCC(c2ccccc2)C(c2ccc(Cl)cc2)=N1)c1cccc(C(F)(F)F)c1]

### Synthesis of 3-(4-chlorophenyl)-N-(naphthalen-2-ylsulfonyl)-4-phenyl-5,6-dihydropyridazine-1 (4H)-carbothioamide (9d)

Following above procedure, starting urea compound (100 mg, 0.198 mmol) was converted in a two-step, one-pot protocol to title compound thiourea 9d (55 mg, 53% yield) as a pale solid. 1H-NMR (800 MHZ; CDCl3): δ 10.48 (s, 1H), 8.78 (s, 1H), 8.08 (d, J=8.7 Hz, 1H), 8.03 (d, J=8.0 Hz, 1H), 7.98 (d, J=8.6 Hz, 1H), 7.92 (d, J=8.1 Hz, 1H), 7.66 (dd, J=8.1, 7.0 Hz, 1H), 7.63-7.61 (m, 3H), 7.34-7.33 (m, 2H), 7.31 (t, J=7.5 Hz, 2H), 7.25 (d, J=9.7 Hz, 1H), 7.06 (d, J=7.8 Hz, 2H), 4.93 (d, J=13.8 Hz, 1H), 4.25 (s, 1H), 3.17 (td, J=13.4, 3.3 Hz, 1H), 2.19-2.10 (m, 2H). 13-C NMR (201 MHz; CDCl3): δ 174.0, 149.1, 139.7, 136.7, 135.5, 135.3, 133.9, 132.0, 129.8, 129.5, 129.3, 128.9, 128.1, 127.9, 127.8, 127.6, 123.8, 39.0, 38.4, 26.4. LRMS 520.0, HRMS (C27H23CIN302S2) [M+H]+found m/z 520.0922, and calcd 520.0920.

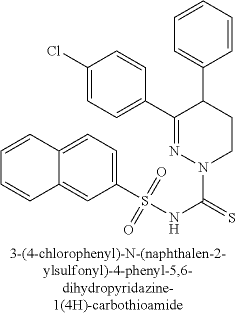 [SMILES: O=S(=O)(NC(=S)N1CCC(c2ccccc2)C(c2ccc(Cl)cc2)=N1)c1ccc2ccccc2c1]

### Synthesis of methyl (E)-3-(4-chlorophenyl)-N-(naphthalen-2-ylsulfonyl)-4-phenyl-5,6-dihydropyridazine-1 (4H)-carbimidothioate (9e)

To a mixture of sulfonylurea compound 9d (1 eq) in toluene (5 mL), POCl₃ (1.5 eq), was added, followed by the addition of N, N-diisopropylethylamine (DIPEA) (2 eq) and the mixture was heated to 115° C. for 3 h under N₂ atmosphere. The reaction mixture was then cooled, and the excess reagents in toluene were evaporated in vacuo. The imidoyl chloride intermediate was dissolved in dioxane (8 ml), to this solution was added dropwise, sodium thiosulfate (2 eq, dissolved in 2 ml water and the reaction was heated to 90° C. Upon completion of reaction as seen by TLC/LCMS, alkylating agent was added dropwise to the reaction mixture and reaction continued until all the thiourea is consumed as seen by TLC/LCMS. The reaction was cooled to room temperature and the organic mixture was the extracted into dichloromethane, washed with brine, and dried over Na₂SO₄. The solvent was removed in vacuo and dried thoroughly to afford a sticky solid. The sticky solid was purified by flash chromatography (20% hexanes in EtOAc) to afford thio-alkylated sulfonyl compounds. Following General procedure, 3-(4-chlorophenyl)-N-(naphthalen-2-ylsulfonyl)-4-phenyl-5,6-dihydropyridazine-1 (4H)-carboxamide (100 mg, 0.19 mmol) was converted in a three-step, one-pot protocol to title compound Methyl-3-(4-chlorophenyl)-N-(naphthalen-2-ylsulfonyl)-4-phenyl-5,6-dihydropyridazine-1 (4H)-carbimidothioate 9e as white solid (30 mg, 29% yield). 1H-NMR (800 MHz; CDCl3): δ 8.51 (s, 1H), 7.98 (d, J=8.6 Hz, 1H), 7.94 (dd, J=19.2, 8.2 Hz, 2H), 7.89 (d, J=7.9 Hz, 1H), 7.70 (d, J=7.8 Hz, 2H), 7.59 (td, J=15.4, 7.5 Hz, 2H), 7.33 (t, J=7.1 Hz, 2H), 7.27 (s, 2H), 7.26 (t, J=1.9 Hz, 1H), 7.14 (d, J=7.7 Hz, 2H), 4.59 (d, J=12.9 Hz, 1H), 4.24 (s, 1H), 3.62 (td, J=13.3, 2.7 Hz, 1H), 2.34 (s, 3H), 2.28-2.13 (m, 2H). 13-C NMR (201 MHz; CDCl3): δ 168.6, 150.3, 141.5, 139.9, 136.2, 134.5, 134.2, 132.2, 129.4, 129.0, 128.3, 128.0, 127.7, 127.3, 126.3, 122.6, 42.3, 37.8, 26.6, 16.8. LRMS 534.1, HRMS (C28H25ClN3O2S2) [M+H]+found m/z 534.1075, and calcd 534.1077.

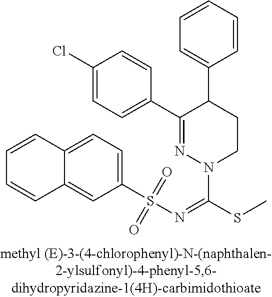 [SMILES: CS/C(=N/S(=O)(=O)c1ccc2ccccc2c1)N1CCC(c2ccccc2)C(c2ccc(Cl)cc2)=N1]

### Synthesis of (E)-N-((3s,5s,7s)-adamantan-1-yl)-3-(4-chlorophenyl)-4-phenyl-N′-((4-(trifluoromethyl)phenyl)sulfonyl)-5,6-dihydropyridazine-1 (4H)-carboximidamide (20)

Following general procedure D, starting material (300 mg) gave final compound (198 mg, 52.6%) as a white solid. LRMS 655.2, HRMS (ESI) m/z: [M+H]+Calcd for (C34 H35 N4 O2 F3 S Cl) 655.2121; Found 655.2130.

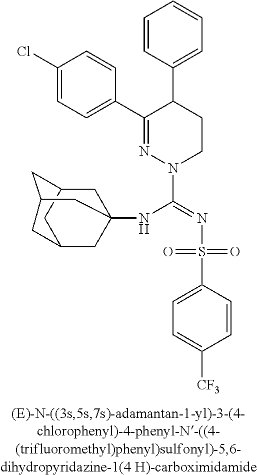 [SMILES: O=S(=O)(/N=C(\NC12CC3CC(CC(C3)C1)C2)N1CCC(c2ccccc2)C(c2ccc(Cl)cc2)=N1)c1ccc(C(F)(F)F)cc1]

### Synthesis of (Z)-4-(3-(4-chlorophenyl)-4-phenyl-N′-((4-(trifluoromethyl)phenyl)sulfonyl)-1,4,5,6-tetrahydropyridazine-1-carboximidamido) butanoic acid (21)

Following general procedure D, starting material (300 mg) gave final compound (212 mg, 61%) as a white solid. LRMS 607.1, HRMS (ESI) m/z: [M+H]+Calcd for (C28 H27 N4 O4 F3 S Cl) 607.1394; Found 607.1396.

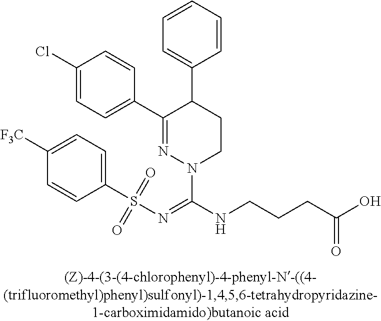 [SMILES: O=C(O)CCCN/C(=N/S(=O)(=O)c1ccc(C(F)(F)F)cc1)N1CCC(c2ccccc2)C(c2ccc(Cl)cc2)=N1]

### Synthesis of (Z)-2-((3-(4-chlorophenyl)-4-phenyl-5,6-dihydropyridazin-1 (4H)-yl)(((4-(trifluoromethyl)phenyl)sulfonyl)imino)methyl) hydrazine-1-carbothioamide (22)

Following general procedure D, starting material (300 mg) gave final compound (56 mg, 16.3%) as a white solid. LRMS 595.1, HRMS (ESI) m/z: [M+H]+Calcd for (C25 H23 N6 O2 F3 S2 Cl) 595.0965; Found 595.0969.

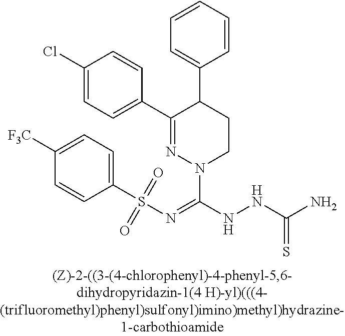 [SMILES: NC(=S)NN/C(=N/S(=O)(=O)c1ccc(C(F)(F)F)cc1)N1CCC(c2ccccc2)C(c2ccc(Cl)cc2)=N1]

### Example 4

### General Procedure D for the Synthesis of methyl (Z)-((3-(4-chlorophenyl)-4-phenyl-5,6-dihydropyridazin-1 (4H)-yl)(((4-(trifluoromethyl)phenyl)sulfonyl)imino)methyl)glycinate (23)

To a mixture of compound, 8c (300 mg, 0.574 mmol) in toluene (2 mL), POCl₃ (107.5 μL, 1.15 mmol), was added, followed by the addition of DIPEA (300 μL, 1.72 mmol) and the mixture was refluxed for 3-4 h. The reaction mixture was then cooled, and toluene was evaporated in vacuo. The crude imidoyl chloride compound was dissolved in dichloromethane (1-2 mL), and to it was added a premixed mixture of Glycine methyl ester hydrochloride (288 mg, 2.3 mmol) in methanol/dichloromethane (1:4, 2 mL) and Et₃N (480 μL, 3.45 mmol) at room temperature dropwise. The mixture was allowed to warm up to room temperature and stirred for 18 hrs. The reaction mixture was extracted into dichloromethane, washed with water, and purified by flash chromatography using hexanes/EtOAC (4:6) followed by trituration with IPA/hexane to afford compound 23 (15 mg, 4.5%) as a white solid.

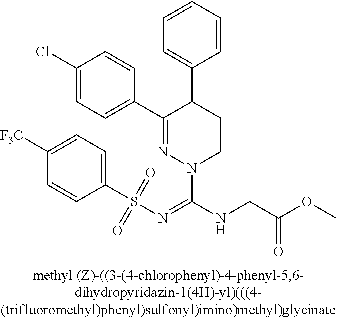 [SMILES: COC(=O)CN/C(=N/S(=O)(=O)c1ccc(C(F)(F)F)cc1)N1CCC(c2ccccc2)C(c2ccc(Cl)cc2)=N1]

### (Z)-2-(3-(4-chlorophenyl)-4-phenyl-N′-((4-(trifluoromethyl)phenyl)sulfonyl)-1,4,5,6-tetrahydropyridazine-1-carboximidamido)acetamide (24)

Following general procedure D, starting material (300 mg) gave final compound (15 mg, 4.6%) as a white solid. LRMS 578.1, HRMS (ESI) m/z: [M+H]+Calcd for (C25 H24 N5 O3 S Cl2) 578.1240; Found 578.1251.

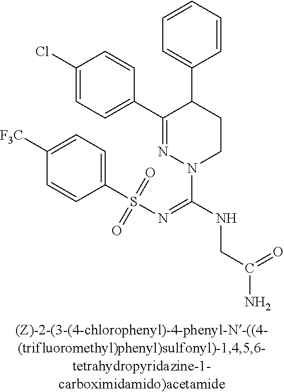 [SMILES: NC(=O)CN/C(=N/S(=O)(=O)c1ccc(C(F)(F)F)cc1)N1CCC(c2ccccc2)C(c2ccc(Cl)cc2)=N1]

### Synthesis of (Z)-2-(3-(4-chlorophenyl)-N′-((4-chlorophenyl)sulfonyl)-4-phenyl-1,4,5,6-tetrahydropyridazine-1-carboximidamido)acetamide (25)

Following general procedure D, starting material (400 mg) gave final compound 25 (250 mg, 52%) as a white solid. LRMS 544.1, HRMS (ESI) m/z: [M+H]+Calcd for (C25 H24 N5 O3 S C12) 544.0977; Found 544.0976.

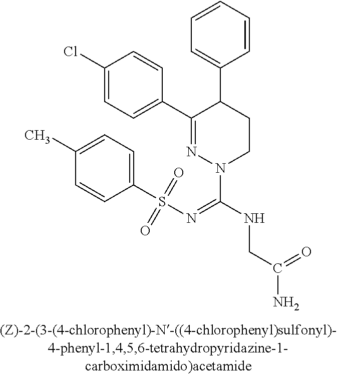 [SMILES: Cc1ccc(S(=O)(=O)/N=C(/NCC(N)=O)N2CCC(c3ccccc3)C(c3ccc(Cl)cc3)=N2)cc1]

### Synthesis of (Z)-2-(3-(4-bromophenyl)-4-phenyl-N′-((4-(trifluoromethyl)phenyl)sulfonyl)-1,4,5,6-tetrahydropyridazine-1-carboximidamido)-3-methylbutanamide (26a-26b)

Following general procedure D, starting material (265 mg) gave final compound first diastereomer 26a (70 mg, 22.5%) as a white solid. LRMS 666.1, HRMS (EST) m/7: [M+H]+Calcd for (C29 H30 Br F3 N5 O3 S) 664.1205; Found 664.1214. and the second diastereomer 26b (76 mg, 24.4%) as a white solid. LRMS 666.1, HRMS (ESI) m/z: [M+H]+Calcd for (C29 H30 Br F3 N5 O3 S) 664.1205; Found 664.1212.

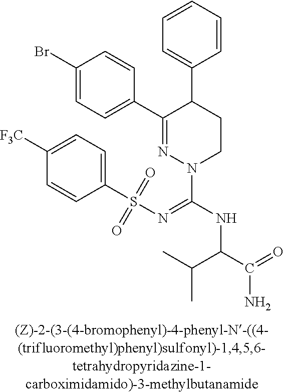 [SMILES: CC(C)C(N/C(=N/S(=O)(=O)c1ccc(C(F)(F)F)cc1)N1CCC(c2ccccc2)C(c2ccc(Br)cc2)=N1)C(N)=O]

### Synthesis of (Z)-2-(3-(4-chlorophenyl)-N′-((4-methoxyphenyl)sulfonyl)-4-phenyl-1,4,5,6-tetrahydropyridazine-1-carboximidamido)-3-methylbutanamide 27a-27b)

Following general procedure D, starting material (200 mg) gave final diastereomer 27a (60 mg, 25%) as a white solid. LRMS 582.2, HRMS (ESI) m/z: [M+H]+Calcd for (C29 H33 N5 O4 S Cl) 582.1942; Found 582.1937. and the second diastereomer 27b (56 mg, 23.3%) as a white solid. LRMS 582.2, HRMS (ESI) m/z: [M+H]+Calcd for (C29 H33 N5 O4 S Cl) 582.1942; Found 582.1945.

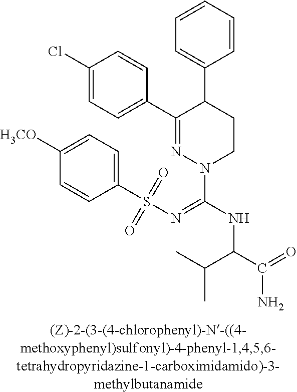 [SMILES: COc1ccc(S(=O)(=O)/N=C(/NC(C(N)=O)C(C)C)N2CCC(c3ccccc3)C(c3ccc(Cl)cc3)=N2)cc1]

### Synthesis of (Z)-2-(3-(4-chlorophenyl)-4-phenyl-N′-(phenylsulfonyl)-1,4,5,6-tetrahydropyridazine-1-carboximidamido)-3-methylbutanamide (28a-28b)

Following general procedure D, starting material (200 mg) gave first diastereomer compound 28a (35 mg, 14.5%) as a white solid. LRMS 552.2, HRMS (ESI) m/z: [M+H]+Calcd for (C28 H31 N5 O3 S Cl) 552.1836; Found 552.1841. and the second diastereomer compound 28b (47 mg, 19.5%) as a white solid. LRMS 552.2, HRMS (ESI) m/z: [M+H]+Calcd for (C28 H31 N5 O3 S Cl) 552.1836; Found 552.1830.

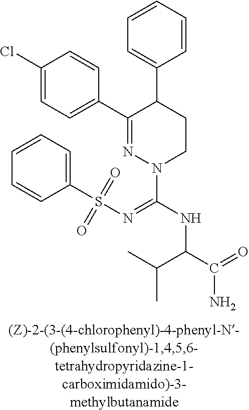 [SMILES: CC(C)C(N/C(=N/S(=O)(=O)c1ccccc1)N1CCC(c2ccccc2)C(c2ccc(Cl)cc2)=N1)C(N)=O]

### Synthesis of (Z)-2-(3-(4-chlorophenyl)-4-phenyl-N′-((4-(trifluoromethyl)phenyl)sulfonyl)-1,4,5,6-tetrahydropyridazine-1-carboximidamido)-3-methylbutanamide (29a-29b)

Following general procedure D, starting material (350 mg) gave first diastereomer compound 29a (120 mg, 29%) as a white solid. 1H NMR (800 MHZ, CDCl₃) δ 8.05 (d, J=7.8 Hz, 2H), 7.92 (s, 1H), 7.69 (d, J=8.5 Hz, 2H), 7.58 (d, J=8.7 Hz, 2H), 7.34-7.27 (m, 4H), 7.25 (d, J=7.5 Hz, 1H), 7.06 (d, J=2.5 Hz, 2H), 6.52 (s, 1H), 5.54 (s, 1H), 4.84 (d, J=17.0 Hz, 1H), 4.20 (d, J=49.4 Hz, 2H), 3.00 (s, 1H), 2.39-2.29 (m, 1H), 2.25-2.12 (m, 1H), 2.03 (dd, J=13.4, 2.3 Hz, 1H), 1.13 (dd, J=39.2, 6.8 Hz, 6H). LRMS 620.2, HRMS (ESI) m/z: [M+H]+Calcd for (C₂₉ H₃₀ N₅ O₃ F₃ S Cl) 620.1710; Found 620.1710. and the second diastereomer compound 29b (108 mg, 26%) as a white solid. 1H NMR (800 MHZ, CDCl₃) δ 8.06 (d, J=8.8 Hz, 2H), 7.79 (d, J=10.1 Hz, 1H), 7.70 (d, J=8.1 Hz, 2H), 7.56 (d, J=8.8 Hz, 2H), 7.34-7.28 (m, 5H), 7.07 (d, J=8.7 Hz, 2H), 6.55 (s, 1H), 5.52 (s, 1H), 4.80 (s, 1H), 4.20 (s, 1H), 4.03 (d, J=13.4 Hz, 1H), 3.13 (s, 1H), 2.35 (s, 1H), 2.06 (dd, J=64.5, 15.3 Hz, 2H), 1.16 (d, J=6.9 Hz, 3H), 1.10 (d, J=6.7 Hz, 3H). LRMS 620.2, HRMS (ESI) m/z: [M+H]+Calcd for (C₂₉ H₃₀ N₅ O₃ F₃ S Cl) 620.1710; Found 620.1705.

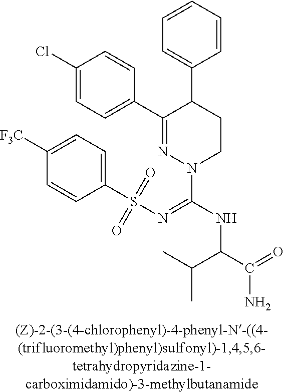 [SMILES: CC(C)C(N/C(=N/S(=O)(=O)c1ccc(C(F)(F)F)cc1)N1CCC(c2ccccc2)C(c2ccc(Cl)cc2)=N1)C(N)=O]

### Synthesis of (Z)-2-(3-(4-chlorophenyl)-N′-((4-chlorophenyl)sulfonyl)-4-phenyl-1,4,5,6-tetrahydropyridazine-1-carboximidamido)-3-methylbutanamide (30a-30b)

Following general procedure D, starting material (400 mg) gave final diastereomeric compound 30a (170 mg, 36%) as a white solid. LRMS 586.2, HRMS (ESI) m/z: [M+H]+Calcd for (C28 H30 N5 O3 S Cl2) 586.1446; Found 586.1446. and the second diastereomer 30b (170 mg, 36%) as a white solid. LRMS 586.2, HRMS (ESI) m/z: [M+H]+Calcd for (C28 H30 N5 O3 S Cl2) 586.1446; Found 586.1445.

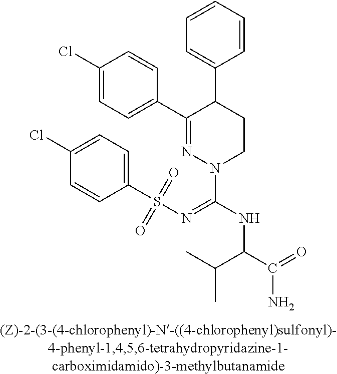 [SMILES: CC(C)C(N/C(=N/S(=O)(=O)c1ccc(Cl)cc1)N1CCC(c2ccccc2)C(c2ccc(Cl)cc2)=N1)C(N)=O]

### Synthesis of (Z)-2-(3-(4-chlorophenyl)-4-phenyl-N′-((4-(trifluoromethyl)phenyl)sulfonyl)-1,4,5,6-tetrahydropyridazine-1-carboximidamido) propenamide (31a-31b)

Following general procedure D, starting material (300 mg) gave first diastereomer compound 31a (98 mg, 28.6%) as a white solid. LRMS 591.8 And second diastereomer compound 31b (58 mg, 17%) as a white solid. LRMS 591.8.

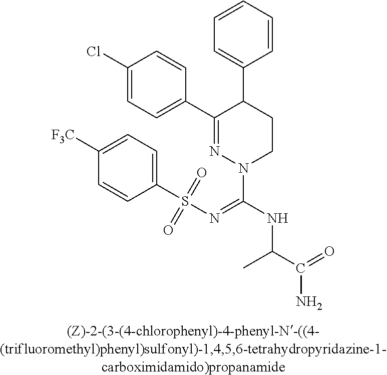 [SMILES: CC(N/C(=N/S(=O)(=O)c1ccc(C(F)(F)F)cc1)N1CCC(c2ccccc2)C(c2ccc(Cl)cc2)=N1)C(N)=O]

### Synthesis of (Z)-2-(3-(4-chlorophenyl)-4-phenyl-N′-((4-(trifluoromethyl)phenyl)sulfonyl)-1,4,5,6-tetrahydropyridazine-1-carboximidamido) butanamide (32a-32b)

Following general procedure D, starting material (300 mg) gave first diastereomer compound 32a (98 mg, 28%) as a white solid. LRMS 605.9 And second diastereomer compound 32b (108 mg, 31%) as a white solid. LRMS 605.9.

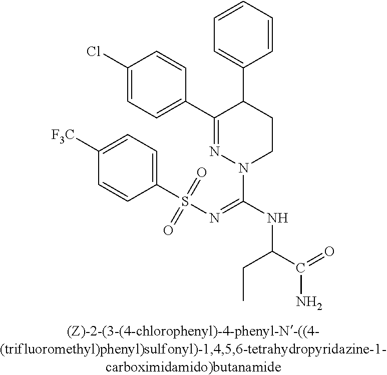 [SMILES: CCC(N/C(=N/S(=O)(=O)c1ccc(C(F)(F)F)cc1)N1CCC(c2ccccc2)C(c2ccc(Cl)cc2)=N1)C(N)=O]

### Synthesis of (Z)-2-(3-(4-chlorophenyl)-4-phenyl-N′-((4-(trifluoromethyl)phenyl)sulfonyl)-1,4,5,6-tetrahydropyridazine-1-carboximidamido)-3-phenylpropanamide (33a-33b)

Following general procedure D, starting material (300 mg) gave first diastereomer compound 33a (110 mg, 28.6%) as a white solid LRMS 667.8 and the second diastereomer compound 33b (105 mg, 28.3%) as a white solid. LRMS 667.8.

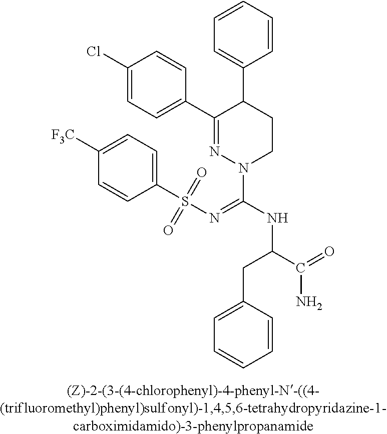 [SMILES: NC(=O)C(Cc1ccccc1)N/C(=N/S(=O)(=O)c1ccc(C(F)(F)F)cc1)N1CCC(c2ccccc2)C(c2ccc(Cl)cc2)=N1]

### Synthesis of methyl ((Z)—((S)-3-(4-chlorophenyl)-4-phenyl-5,6-dihydropyridazin-1 (4H)-yl)(((4-(trifluoromethyl)phenyl)sulfonyl)imino)methyl)-D-valinate (34a-34b)

Following general procedure, starting material (300 mg) gave first diastereomer compound 34a (60 mg, 16.5%) as a white solid LRMS 634.8 and the second diastereomer compound 34b (51 mg, 14%) as a white solid. LRMS 634.9.

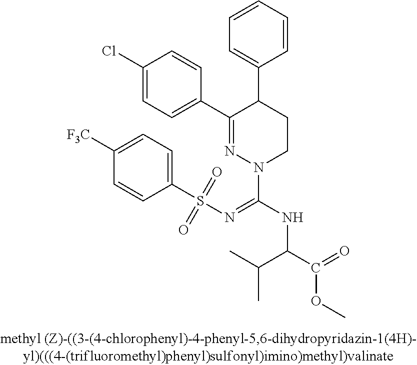 [SMILES: COC(=O)C(N/C(=N/S(=O)(=O)c1ccc(C(F)(F)F)cc1)N1CCC(c2ccccc2)C(c2ccc(Cl)cc2)=N1)C(C)C]

Table # below summarizes in vitro data for several compounds based on the structure:

| | | Functional | | | | iNOS |
|---|---|---|---|---|---|---|
| | Kᵢ (CB₁) | GTPγS binding | | | | inhibition % |
| Compound name | in nM | Brain/CB1, IC₅₀ (nM) | PSA^{a} | cLogP^{a} | [M + H]⁺ | Response |
| 23 | 4.37 | 8.1 | 100.43 | 6.51212 | 592.8 | N.D |
| 24 | 10 | 33 | 117.22 | 5.38892 | 577.8 | N.D |
| 25 | 41.1 | 207 | 117.22 | 5.21892 | 544.1 | N.D |
| 26a | ND | >1000 | 117.22 | 6.77592 | 586.2 | N.D |
| 26b | ND | 9.5 | 117.22 | 6.77592 | 586.2 | N.D |
| 27a | ND | >1000 | 126.45 | 5.66192 | 582.2 | N.D |
| 27b | ND | 51 | 126.45 | 5.66192 | 582.2 | N.D |
| 28a | ND | >1000 | 117.22 | 5.74292 | 552.2 | N.D |
| 28b | ND | 336 | 117.22 | 5.74292 | 552,2 | N.D |
| 29a | 13.2 | 389 | 117.22 | 6.62592 | 619.9 | N.D |
| 29b | 2.7 | 10 | 117.22 | 6.62592 | 619.9 | N.D |
| 30a | 906.6 | >1000 | 117.22 | 6.45592 | 586.2 | N.D |
| 30b | 18.2 | 39 | 117.22 | 6.45592 | 586.2 | N.D |
| 31a | 96.9 | 863 | 117.22 | 5.69792 | 591.8 | N.D |
| 31b | 8.05 | 33 | 117.22 | 5.69792 | 591.8 | N.D |
| 32a | 785 | ND | 117.22 | 6.22692 | 605.9 | N.D |
| 32b | 77.4 | 174 | 117.22 | 6.22692 | 605.9 | N.D |
| 33a | 66.8 | 423 | 117.22 | 7.11592 | 667.8 | N.D |
| 33b | 6.13 | 12 | 117.22 | 7.11592 | 667.8 | N.D |
| 34a | 46.4 | 41 | 100.43 | 7.74912 | 634.8 | N.D |
| 34b | 2.71 | 17 | 100.43 | 7.74912 | 634.9 | N.D |
| 10a | 76 | 138 | 100.48 | 6.569 | 528.3 | 39 |
| 11a | 210 | ND | 100.48 | 6.569 | 560.2 | N.D |
| 12a | 45 | ND | 100.48 | 6.569 | 608.0 | N.D |
| 10b | 15 | 93 | 100.48 | 6.739 | 562.3 | 64 |
| 10bE1 | ND | >1000 | 100.48 | 6.739 | ND | N.D |
| 10bE2 | ND | 34 | 100.48 | 6.739 | ND | N.D |
| 11b | 86.7 | 125 | 100.48 | 6.739 | 594.3 | 56 |
| 12b | 114 | ND | 100.48 | 6.739 | 642.1 | 64 |
| 10c | 28.3 | 105 | 100.48 | 6.739 | 562.1 | N.D |
| 11c | 234 | ND | 100.48 | 6.739 | 594.1 | N.D |
| 12c | ND | ND | 100.48 | 6.739 | 642.1 | N.D |
| 10d | 56.5 | 104 | 109.71 | 6.884 | 578.1 | 55 |
| 11d | 151 | ND | 109.71 | 6.884 | 610.1 | N.D |
| 12d | 194 | ND | 109.71 | 6.884 | 658.1 | N.D |
| 10e | 77 | 338 | 124.27 | 5.289 | 519.1 | 43 |
| 11e | 138 | ND | 124.27 | 5.289 | 551.1 | N.D |
| 12e | 179 | ND | 124.27 | 5.289 | 599.1 | N.D |
| 10g | 128.5 | 357 | 100.48 | 5.856 | 494.2 | N.D |
| 11g | 1600 | ND | 100.48 | 5.856 | 525.8 | N.D |
| 12g | 2800 | ND | 100.48 | 5.856 | 573.8 | N.D |
| 10f | 31.4 | 132 | 100.48 | 7.03 | 544.2 | N.D |
| 11f | 590 | ND | 100.48 | 7.03 | 576.2 | N.D |
| 12f | 317 | ND | 100.48 | 7.03 | 624.1 | N.D |
| 10h | 105 | 357 | 109.71 | 5.775 | 523.9 | N.D |
| 11h | 1070 | ND | 109.71 | 5.775 | 555.8 | N.D |
| 12h | 1100 | ND | 109.71 | 5.775 | 603.8 | N.D |
| 10i | 43 | 118 | 100.48 | 6.979 | 620.1 | N.D |
| 10j | 59.3 | 182 | 100.48 | 5.999 | 512.2 | N.D |
| 10k | 50.6 | 125 | 100.48 | 6.739 | 562.2 | N.D |
| 101 | 84 | >1000 | 100.48 | 6.355 | 508.2 | N.D |
| 10m | 23 | 151 | 100.48 | 7.577 | 589.9 | 55 |
| 10n | 44 | 211 | 100.48 | 7.323 | 587.9 | 55 |
| 10o | 10 | 18 | 100.48 | 7.976 | 603.9 | 66 |
| 10oE1 | 351.5 | 222 | 100.48 | 7.976 | ND | N.D |
| 10oE2 | 7.3 | 11 | 100.48 | 7.976 | ND | N.D |
| 10p | 9.6 | 13 | 100.48 | 8.46 | 659.9 | 36 |
| 10g | 212 | ND | 100.48 | 9.398 | 681.9 | N.D |
| 10r | 33 | 197 | 100.48 | 8.174 | 623.9 | N.D |
| 10s | 20.5 | 19 | 100.48 | 8.887 | 657.9 | 24 |
| 10t | 7.4 | 25 | 100.48 | 8.317 | 642.1 | N.D |
| 10tE1 | ND | 51 | 100.48 | 8.317 | ND | N.D |
| 10tE2 | ND | 18 | 100.48 | 8.317 | ND | N.D |
| 10u | 27.25 | 107 | 100.48 | 8.317 | 642.2 | N.D |
| 10v | 7 | 26 | 112.84 | 6.677 | 625.2 | N.D |
| 10vE1 | 11.5 | 107 | 112.84 | 6.677 | ND | N.D |
| 10vE2 | 5 | 24 | 112.84 | 6.677 | ND | N.D |
| 10w | 140 | 234 | 100.48 | 7.82 | 630.1 | N.D |
| 11i | 99.7 | 640 | 100.48 | 7.268 | 607.8 | 66 |
| 10x | 20.34 | 35 | 100.48 | 8.126 | 648.0 | N.D |
| 10y | 57.2 | 92 | 100.48 | 6.889 | 605.9 | N.D |
| 10z | 8.25 | 49 | 100.48 | 8.467 | 685.9 | N.D |
| 10za | 9.18 | 26 | 100.48 | 8.61 | 703.9 | N.D |
| 10zb | 75.6 | 478 | 100.48 | 7.406 | 588.2 | N.D |
| 10zc | 120 | ND | 100.48 | 6.169 | 546.2 | N.D |
| 11j | 93 | >1000 | 100.48 | 6.169 | 578.0 | N.D |
| 12i | 110 | ND | 100.48 | 6.169 | 625.9 | N.D |
| 10zd | 26 | 115 | 100.48 | 7.747 | 626.2 | N.D |
| 10ze | 58.6 | 450 | 100.48 | 7.89 | 644.2 | N.D |
| 10zf | 41.74 | 209 | 112.84 | 6.107 | 609 | N.D |
| 13a | 24.6 | ND | 153.37 | 3.973 | 561.9 | N.D |
| 14 | 5 | ND | 129.58 | 5.423 | 604.8 | N.D |
| 18 | 536 | ND | 103.72 | 6.508 | 618.9 | N.D |
| 19 | 383 | ND | 138.81 | 7.976 | 662.8 | N.D |
| 13b | 19.8 | 325 | 110.28 | 6.215 | 559.2 | N.D |
| 15 | 34.1 | 287 | 86.49 | 7.956 | 584.2 | N.D |
| 16 | 26 | 104 | 86.49 | 7.665 | 601.9 | 51 |
| 17 | 117 | ND | 86.49 | 7.106 | 587.9 | N.D |
| 9c | ND | ND | 85.56 | 4.901 | 495.1 | N.D |
| 9a | ND | ND | 61.77 | 6.181 | 504.1 | N.D |
| 9b | ND | ND | 61.77 | 6.351 | 538.1 | N.D |
| 9d | ND | ND | 61.77 | 6.642 | 520.1 | N.D |
| 9e | ND | ND | 62.1 | 7.214 | 533.9 | N.D |
| 20 | 80.9 | ND | 74.13 | 8.695 | 654.9 | N.D |
| 21 | 579 | ND | 111.43 | 6.5492 | 606.8 | N.D |
| 22 | 575 | ND | 112.18 | 6.899 | 594.8 | N.D |

^{a}Theoretical values

Rotation values for some compounds are as below:

$$10 vE 1 = [ {α}_{D}^{25} ] = + 0.047 ° ( c 0.1 , CHCl 3 )$$

$$10 vE 2 = [ {α}_{D}^{25} ] = - 0.047 ° ( c 0.1 , CHCl 3 )$$

$$10 bE 1 = [ {α}_{D}^{25} ] = + 0.1 ° ( c 0.1 , CHCl 3 )$$

$$10 oE 1 = [ {α}_{D}^{25} ] = + 0.032 ° ( c 0.1 , CHCl 3 )$$

$$10 tE 1 = [ {α}_{D}^{25} ] = + 0.016 ° ( c 0.1 , CHCl 3 )$$

In view of the many possible embodiments to which the principles of the disclosed invention may be applied, it should be recognized that the illustrated embodiments are only preferred examples of the invention and should not be taken as limiting the scope of the invention.

1. A compound, or a pharmaceutically acceptable salt or ester thereof, having a structure of: 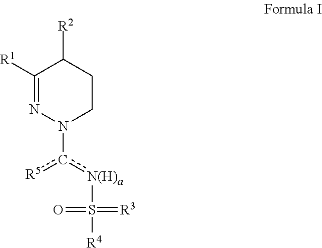
wherein R¹ and R² are each independently aryl, substituted aryl, heteroaryl, or substituted heteroaryl;
R³ is O or NH;
R⁴ is aryl, substituted aryl, heteroaryl, substituted heteroaryl, or N(R¹⁰)(R¹¹), wherein R¹⁰ and R¹¹ are each independently H, optionally-substituted alkyl, optionally-substituted cycloalkyl, halogen, cyano, nitro, hydroxy, optionally-substituted alkoxy, amino, aminocarbonyl, optionally-substituted sulfonyl, optionally-substituted aryl, optionally-substituted heteroaryl, optionally-substituted carboxyl, acyl, optionally-substituted alkenyl, optionally-substituted alkynyl, optionally-substituted phosphonyl, optionally-substituted phosphinyl, aralkyl, or optionally-substituted thiol;
R⁵ is =S, —SR¹², —SeR¹², ═O, —OR¹², —NR¹⁴R¹⁵, —S(O)R¹², —S(O₂)R¹², —Se(O)R¹²—Se(O₂)R¹², or 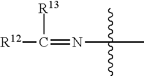
wherein R¹² is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl;
R¹³ is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, allenyl, substituted alkynyl, phosphonyl, substituted phosphonyl, phosphinyl, substituted phosphinyl, boronate, substituted boronate, silyl, substituted silyl, imino, thiol, substituted thiol, selenol, substituted selenol, acyl, or nitrate (ONO₂);
R¹⁴ is H, is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl;
R¹⁵ is H, is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, or acyl; or
R¹⁴ and R¹⁵ together form a cyclic ring, a substituted cyclic ring, a heterocyclic ring or a substituted heterocyclic ring;
provided that R⁵ is not —N(H)(methyl), and R⁵ is not ═O when R⁴ is 4-chlorophenyl, 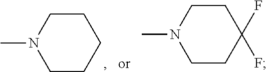
each bond represented by [symbol] is a single or double bond as needed to satisfy valence requirements; and
a is 0 or 1 as needed to satisfy valence requirements.

2. The compound of claim 1, wherein R¹ and R² are each independently phenyl or substituted phenyl.

3. The compound of claim 1, wherein R¹ is halogen-substituted phenyl and R² is phenyl.

4. The compound of claim 1, having a structure of: 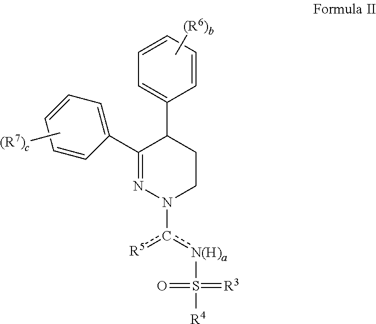
wherein R³, R⁴, R⁵, and subscript a are the same as in formula I;
R⁶ and R⁷ are each independently alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, or substituted thiol;
b is 0 to 5; and
c is 0 to 5.

5. The compound of claim 4, wherein b is 0 and c is 1.

6. The compound of claim 4, wherein R⁷ is halogen.

7. The compound of claim 1, having a structure of: 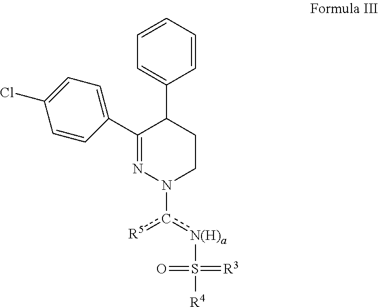
wherein R³, R⁴, R⁵, and subscript a are the same as in formula I.

8. The compound of claim 1, having a structure of: 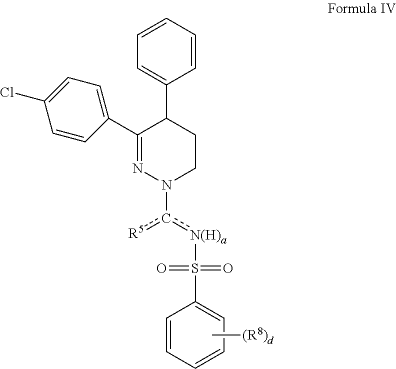
wherein R⁵ and subscript a are the same as in formula I; and
R⁸ is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, or substituted thiol; and
d is 0 to 5.

9. The compound of claim 1, wherein R³ is O.

10. The compound of claim 1, wherein R⁴ is aryl or substituted aryl.

11. (canceled)

12. The compound of claim 10, wherein R⁴ is phenyl, halogen-substituted phenyl, halogenated alkyl-substituted phenyl, alkoxy-substituted phenyl, halogenated alkoxy-substituted phenyl, cyano-substituted phenyl, or naphthyl.

13. The compound of claim 1, wherein R⁵ is O.

14. The compound of claim 1, wherein R⁵ is S.

15. The compound of claim 1, wherein R⁵ is 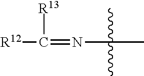
wherein R¹² is amino, substituted amino, acyl, or acetamido.

16. The compound of claim 1, wherein R⁵ is 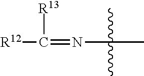
wherein R¹³ is alkyl, substituted thiol, substituted selenol, acetyl, 2-iminopiperidinyl, propionyl, cinnamoyl, guanidino, substituted guanidino, 2-iminopyrrolidinyl, or hydrazine.

17. The compound of claim 15, wherein R¹² is —NH₂.

18. The compound of claim 17, wherein R¹³ is alkyl.

19. The compound of claim 1, wherein R⁵ is —NR¹⁴R¹⁵, wherein R¹⁴ is H and R¹⁵ is substituted alkyl or substituted heteroaryl.

20. The compound of claim 1, wherein R⁵ is —NR¹⁴R¹⁵, wherein R¹⁴ is H and R¹⁵ is —(CHR²⁰)ₙC(═O) OR²⁰, —(CHR²⁰)ₙC(═O) R²⁰, —(CHR²⁰)ₙC(═O)NHR²⁰, or —(CHR²⁰)ₙSO₂NHR²⁰, wherein n is 0 to 6, and R²⁰ is H or alkyl.

21. The compound of claim 8, having a structure of: 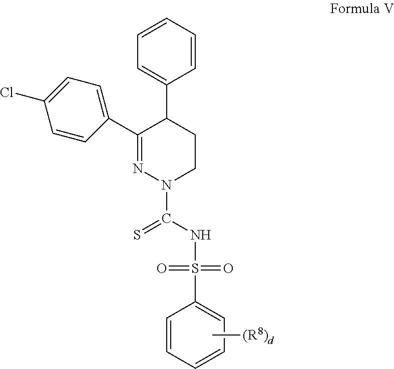
wherein R⁸ and subscript d are the same as in formula IV.

22. The compound of claim 8, having a structure of: 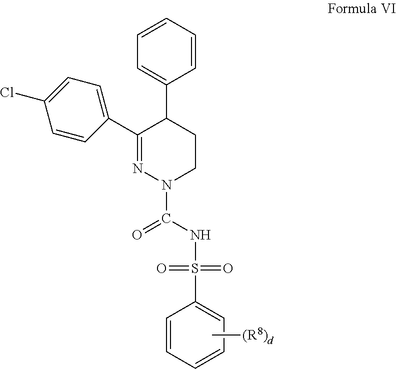
wherein R⁸ and subscript d are the same as in formula IV.

23. The compound of claim 8, having a structure of: 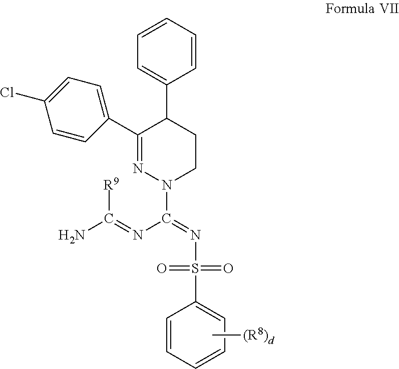
wherein R⁸ and subscript d are the same as in formula IV; and
R⁹ is alkyl, substituted alkyl, cycloalkyl, substituted cycloalkyl, halogen, cyano, nitro, hydroxy, alkoxy, substituted alkoxy, amino, substituted amino, sulfonyl, substituted sulfonyl, aryl, substituted aryl, heteroaryl, substituted heteroaryl, carboxyl, substituted carboxyl, acyl, alkenyl, substituted alkenyl, alkynyl, substituted alkynyl, phosphonyl, substituted phosphonyl, phosphinyl, substituted phosphinyl, boronate, substituted boronate, silyl, substituted silyl, imino, thiol, substituted thiol, selenol, or substituted selenol.

24. The compound of claim 8, wherein R⁸ is halogen, halogenated alkyl, lower alkyl, nitro, naphthyl, halogenated alkoxy, cyano, or alkoxy.

25. The compound of claim 8, wherein R⁸ is —Cl, —I, —Br, —F, —CH₃, -naphthyl, —NO₂, —CF₃, —OCF₃. —CN, or —OCH₃.

26. The compound of claim 8, wherein d is 1, R⁸ is in the para position, and is —CF₃ or —OCF₃.

27. The compound of claim 23, wherein R⁹ is alkyl,

28. The compound of claim 23, wherein R⁹ is heteroaryl.

29. The compound of claim 23, wherein R⁹ is substituted aryl.

30. A compound, or a pharmaceutically acceptable salt or ester thereof, having a structure of:

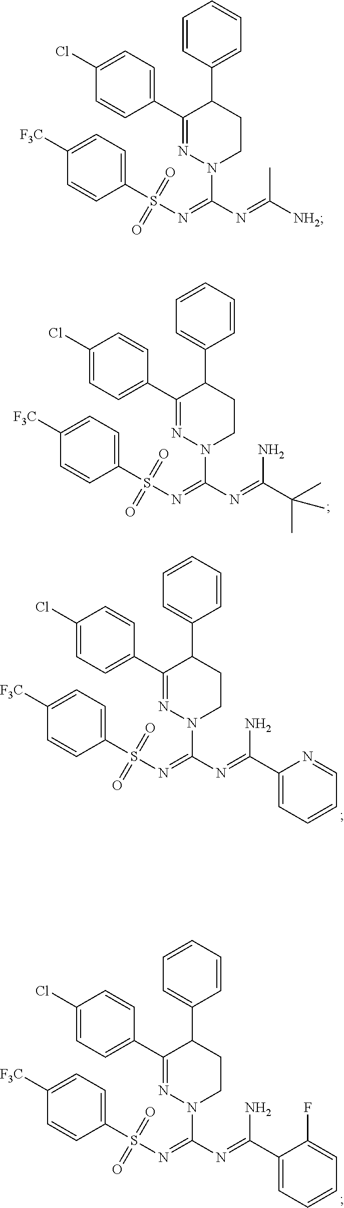 [SMILES: C/C(N)=N\C(=N\S(=O)(=O)c1ccc(C(F)(F)F)cc1)N1CCC(c2ccccc2)C(c2ccc(Cl)cc2)=N1 ; CC(C)(C)/C(N)=N/C(=N/S(=O)(=O)c1ccc(C(F)(F)F)cc1)N1CCC(c2ccccc2)C(c2ccc(Cl)cc2)=N1 ; N/C(=N\C(=N\S(=O)(=O)c1ccc(C(F)(F)F)cc1)N1CCC(c2ccccc2)C(c2ccc(Cl)cc2)=N1)c1ccccn1 ; N/C(=N\C(=N\S(=O)(=O)c1ccc(C(F)(F)F)cc1)N1CCC(c2ccccc2)C(c2ccc(Cl)cc2)=N1)c1ccccc1F]

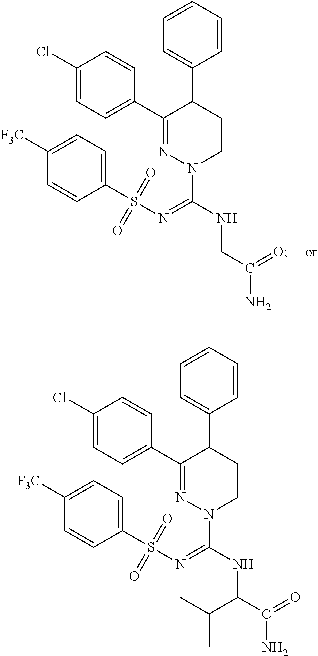 [SMILES: NC(=O)CN/C(=N/S(=O)(=O)c1ccc(C(F)(F)F)cc1)N1CCC(c2ccccc2)C(c2ccc(Cl)cc2)=N1 ; CC(C)C(N/C(=N/S(=O)(=O)c1ccc(C(F)(F)F)cc1)N1CCC(c2ccccc2)C(c2ccc(Cl)cc2)=N1)C(N)=O]

31. The compound of claim 7, wherein the compound is an (−) enantiomer.

32. (canceled)

33. A pharmaceutical composition comprising a compound of claim 1, wherein the composition is in unit dosage form.

34. A method for reducing food intake and body weight, reversing insulin and leptin resistance, revering hepatic steatosis or improving dyslipidemia in a subject, comprising administering a therapeutically effective amount of a compound of claim 1 to a subject in need thereof.

35. A method for treating obesity, diabetes, non-alcoholic fatty liver disease, alcoholic fatty liver disease, insulin resistance, cirrhosis, liver cancer, dyslipidemias that predispose to arteriosclerotic heart disease, diabetic nephropathy, gout, fibrosis, or obesity-induced chronic kidney disease in a subject, comprising administering a therapeutically effective amount of a compound of claim 1 to a subject in need thereof.

36. A method for treating a co-morbidity of obesity in a subject, comprising administering a therapeutically effective amount of a compound of claim 1 to a subject in need thereof, wherein the co-morbidity is diabetes, Metabolic Syndrome, dementia, heart disease, cancer, hypertension, gallbladder disease, gastrointestinal disorders, menstrual irregularities, degenerative arthritis, venous statis ulcer, pulmonary hypoventilation syndrome, sleep apnea, snoring, coronary artery disease, arterial sclerotic disease, pseudotumor cerebri, accident proneness, increased risks with surgeries, osteoarthritis, high cholesterol, or increased incidence of malignancies of the liver, ovaries, cervix, uterus, breasts, prostrate, or gallbladder.

37. A method of preventing or reversing the deposition of adipose tissue in a subject, or preventing or reversing ectopic deposition of fat in a subject comprising administering a therapeutically effective amount of a compound of claim 1 to a subject in need thereof.

38. A method for treating idiopathic pulmonary fibrosis, Hermansky-Pudlak syndrome associated pulmonary fibrosis, radiation induced pulmonary fibrosis, scleroderma associated organ fibrosis, skin fibrosis, kidney fibrosis, liver fibrosis, chronic graft versus host disease (cGvHD) or any combination thereof in a subject, comprising administering a therapeutically effective amount of a compound of claim 1 to a subject in need thereof.
